(19) United States
(12) Patent Application Publication
(10) Pub. No.: US 2023/0381375 A1
(43) Pub. Date: Nov. 30, 2023

(54) HYDROGEL STRUCTURE, METHOD FOR PRODUCING HYDROGEL STRUCTURE, AGENT, AND TRANSPLANTATION METHOD

(71) Applicants: CellFiber Co., Ltd., Tokyo (JP); SAPPORO MEDICAL UNIVERSITY, Sapporo-shi Hokkaido (JP)

(72) Inventors: Kanna NAGAISHI, Sapporo-shi (JP); Kazuhiro IKEDA, Tokyo (JP)

(73) Assignees: CellFiber Co., Ltd., Tokyo (JP); SAPPORO MEDICAL UNIVERSITY, Sapporo-shi Hokkaido (JP)

(21) Appl. No.: 18/034,564

(22) PCT Filed: Oct. 29, 2021
(86) PCT No.: PCT/JP2021/040183

§ 371 Date: Apr. 28, 2023

(30) Foreign Application Priority Data
Oct. 30, 2020 (JP) 2020-183302

(51) Int. Cl. A61L 27/38; A61L 27/20; A61L 27/24; C12N 5/00; A61L 27/36; A61L 27/52; A61P 1/04

(52) U.S. Cl. CPC A61L 27/3834; A61L 27/20; A61L 27/24; C12N 5/0012; A61L 27/3641; A61L 27/52; A61P 1/04; A61L 2300/64

(57) ABSTRACT

Provided is a novel structure containing mesenchymal stem cells that can be used in various applications. A hydrogel fiber (10) comprises a hydrogel (14) that contains mesenchymal stem cells.

### TECHNICAL FIELD

The present invention relates to a hydrogel structure encapsulating mesenchymal stem cells, a method for producing a hydrogel structure, and an agent and a transplantation method related thereto.

### BACKGROUND ART

Mesenchymal stem cells (MSCs) are undifferentiated cells that can be collected and isolated from an umbilical cord, placenta, bone marrow, amnion, dental pulp, or adipose. Mesenchymal stem cells have the ability to differentiate into various mesodermal tissues, such as cells constituting bone marrow stroma, adipocytes, osteocytes, chondrocytes, muscle cells, and tendon, and are expected to be applied in various fields including the medical field.

Non Patent Literature 1 below discloses an experiment of administering, through the tail vein, mesenchymal stem cells (MSCs) to rats in which enteritis has been induced by dextran sulfate sodium (DSS). It is described that an effect of promoting recovery from enteritis is obtained as a result.

Furthermore, Patent Literature 1 below discloses an experiment of administering a supernatant of a culture of bone marrow-derived mesenchymal stem cells (MSCs) to rats in which enteritis has been induced by dextran sulfate sodium (DSS). It is described that an effect of promoting recovery from enteritis is obtained as a result.

### CITATION LIST

### Patent Literature

Patent Literature 1: JP 6132459 B2

### Non Patent Literature

Non Patent Literature 1: “Myogenic lineage differentiated mesenchymal stem cells enhance recovery from dextran sulfate sodium-induced colitis in the rat”; J Gastroenterol (2011) 46:143-152

### SUMMARY

Regarding the applications in treatment and prevention, it is known that MSCs have the ability to accumulate in a diseased site, and intravenous injection of mesenchymal stem cells is effective on a diseased site. However, in Non Patent Literature 1, it is impossible to control whether the MSCs migrate to the tissue intended for the prevention and treatment. In addition, when cultured cells are transplanted into a biological body, the cells are generally attacked by immune cells, which is also problematic.

Moreover, research on MSCs is still developing, leaving room for further development beyond the applications in treatment and prevention.

Therefore, a novel structure containing mesenchymal stem cells is provided, which can be applied in a variety of uses.

According to one aspect, a hydrogel fiber includes a hydrogel encapsulating mesenchymal stem cells.

According to one preferred aspect, the hydrogel fiber includes: the hydrogel; and a base material and the mesenchymal stem cells that are provided inside the hydrogel.

According to one preferred aspect, the base material contains collagen, laminin, fibronectin or a liquid medium, or a combination thereof.

According to one preferred aspect, the mesenchymal stem cells are umbilical cord-derived, placenta-derived, bone marrow-derived, amnion-derived, dental pulp-derived or adipose-derived mesenchymal stem cells.

According to one preferred aspect, the hydrogel contains calcium alginate or barium alginate.

According to one preferred aspect, the hydrogel fiber is for regulation of gene expression of a factor that is expressed by the mesenchymal stem cells.

According to one preferred aspect, the hydrogel fiber is for transplantation.

According to one preferred aspect, the hydrogel fiber is for at least one of suppression of fibrogenesis, suppression of inflammatory cell infiltration, and tissue repair and regeneration.

According to one preferred aspect, the hydrogel fiber is for treating enteritis or preventing enteritis.

According to one preferred aspect, an agent for treating enteritis or for preventing enteritis, the agent comprising a supernatant of a culture medium in which the mesenchymal stem cells are cultured in a state of being encapsulated in the above hydrogel fiber.

According to one aspect, a transplantation method comprising: applying the above hydrogel fiber inside a biological body.

According to one aspect, a method for producing a hydrogel fiber, the method including: mixing mesenchymal stem cells and a base material; and embedding the mixture in a hydrogel.

### BRIEF DESCRIPTION OF DRAWINGS

FIG. 1 is a schematic diagram illustrating a structure of a hydrogel fiber according to an embodiment.

FIG. 2 is a schematic diagram illustrating a cross-section structure of the hydrogel fiber according to an embodiment.

FIG. 3 is a schematic diagram illustrating an example of an apparatus for producing a hydrogel fiber.

FIG. 4 shows graphs of the results of measuring various expression factors related to the mRNA of mesenchymal stem cells encapsulated in hydrogel fibers in Examples 1-1 and 1-2.

FIG. 5 shows a graph of the results of measuring a tissue-repair factor (TGF-β1) secreted from the mesenchymal stem cells encapsulated in the hydrogel fibers in Examples 1-1 and 1-2.

FIG. 6 shows graphs of the results of measuring various expression factors related to the mRNA of mesenchymal stem cells encapsulated in hydrogel fibers in Examples 2-1 to 2-4.

FIG. 7 shows a graph of the results of measuring a tissue-repair factor (TGF-β1) of the mesenchymal stem cells encapsulated in the hydrogel fibers in Examples 2-1 to 2-4.

FIG. 8 shows graphs of the results of measuring various expression factors related to the mRNA of mesenchymal stem cells encapsulated in hydrogel fibers in Examples 3-1 to 3-3.

FIG. 9 shows a graph of the results of measuring a tissue-repair factor (TGF-β1) of the mesenchymal stem cells encapsulated in the hydrogel fibers in Examples 3-1 to 3-3.

FIG. 10 shows graphs of the results of measuring various expression factors related to the mRNA of mesenchymal stem cells encapsulated in hydrogel fibers in Examples 4-1 and 4-2.

FIG. 11 show a graph of the results of measuring a tissue-repair factor (TGF-β1) secreted from the mesenchymal stem cells encapsulated in the hydrogel fibers in Examples 4-1 and 4-2.

FIG. 12 shows a graph of the results of measuring a vascular endothelial growth factor (VEGF) secreted from the mesenchymal stem cells encapsulated in the hydrogel fibers in Examples 4-1 and 4-2.

FIG. 13 shows a graph of the results of measuring a factor (PGE2) secreted from the mesenchymal stem cells encapsulated in the hydrogel fibers in Examples 4-1 and 4-2.

FIG. 14 is a diagram for describing schedules for treatment by the hydrogel fiber in Example 1-1 using TNBS enteritis model mice.

FIG. 15 shows a graph of body weight changes in the TNBS enteritis model mice that have received the various treatments.

FIG. 16 shows a graph of changes in disease activity indices (DAIs) for the TNBS enteritis model mice that have received the various treatments.

FIG. 17 shows a graph of changes in intestinal wet weights in the TNBS enteritis model mice that have received the various treatments.

FIG. 18 shows histopathological images (hematoxylin-eosin staining) of proximal colons of the TNBS enteritis model mice that have received the various treatments.

FIG. 19 is a diagram for describing schedules for treatments by the hydrogel fibers in Example 2-1, 2-2, and 2-4 using naive T cell transfer enteritis model mice.

FIG. 20 shows a graph of body weight changes in the naive T cell transfer enteritis model mice that have received the various treatments.

FIG. 21 shows a graph of changes in disease activity indices (DAIs) for the naive T cell transfer enteritis model mice that have received the various treatments.

FIG. 22 shows a graph of changes in intestinal wet weights in the naive T cell transfer enteritis model mice that have received the various treatments.

FIG. 23 shows a graph of the results of measuring neutrophil gelatinase-associated lipocalin in stools of the naive T cell transfer enteritis model mice that have received the various treatments.

FIG. 24 shows photographs of the states in which the hydrogel fibers transplanted into the naive T cell transfer enteritis model mice are extracted.

FIG. 25 is a diagram for describing schedules for treatments by the hydrogel fibers in Example 3-1, 3-2, and 3-3 using naive T cell transfer enteritis model mice.

FIG. 26 shows a graph of body weight changes in the naive T cell transfer enteritis model mice that have received the various treatments.

FIG. 27 shows a graph of changes in disease activity indices (DAIs) for the naive T cell transfer enteritis model mice that have received the various treatments.

FIG. 28 shows a graph of changes in intestinal wet weights in the naive T cell transfer enteritis model mice that have received the various treatments.

FIG. 29 shows a graph of changes in spleen weights in the naive T cell transfer enteritis model mice that have received the various treatments.

FIG. 30 shows a graph of the results of measuring neutrophil gelatinase-associated lipocalin in stools of the naive T cell transfer enteritis model mice that have received the various treatments.

FIG. 31 is a diagram for describing schedules for treatment by the hydrogel fiber in Example 4-1 using DSS enteritis model mice.

FIG. 32 shows a graph of body weight changes in the DSS enteritis model mice that have received the various treatments.

FIG. 33 shows a graph of changes in disease activity indices (DAIS) for the DSS enteritis model mice that have received the various treatments.

FIG. 34 shows graphs of the results of measuring various expression factors related to the mRNA of the mesenchymal stem cells encapsulated in the hydrogel fibers in Examples 1-1 and 1-2.

FIG. 35 shows a graph of the concentrations of prostaglandin E2 secreted from the mesenchymal stem cells encapsulated in the hydrogel fibers in Examples 1-1 and 1-2.

FIG. 36 is a diagram for describing analysis of changes in cell phenotypes in the macrophage cell line RAW264.7 induced with LPS by humoral factors derived from the mesenchymal stem cells in Examples 1-1 and 1-2.

FIG. 37 is a diagram for describing analysis of a cellular protection effect of humoral factors derived from the mesenchymal stem cells in Examples 1-1 and 1-2 on the intestinal epithelial cell line IEC-6 induced with TNFα.

FIG. 38 shows graphs of the results of measuring various expression factors related to the mRNA of the mesenchymal stem cells encapsulated in the hydrogel fibers in Examples 2-1 to 2-4.

FIG. 39 shows a graph of the concentrations of prostaglandin E2 secreted from the mesenchymal stem cells encapsulated in the hydrogel fibers in Examples 2-1 to 2-4.

FIG. 40 is a diagram for describing analysis of changes in cell phenotypes in the macrophage cell line RAW264.7 induced with LPS by humoral factors derived from the mesenchymal stem cells in Examples 2-1 to 2-4.

FIG. 41 shows micrographs of histopathological images of large intestines acquired after transplanting the mesenchymal stem cells in Examples 2-A and 2-B and Reference Examples 2-1, 2-A, and 2-5.

FIG. 42 shows graphs of the expression levels of inflammatory cytokines in intestinal tissues acquired after transplanting the mesenchymal stem cells in Examples 2-A and 2-B and Reference Examples 2-1, 2-A, and 2-5.

FIG. 43 shows graphs of the results of measuring various expression factors related to the mRNA of the mesenchymal stem cells encapsulated in the hydrogel fibers in Examples 3-1 to 3-3.

FIG. 44 shows a graph of the concentrations of prostaglandin E2 secreted from the mesenchymal stem cells encapsulated in the hydrogel fibers in Examples 3-1 to 3-3.

FIG. 45 is a diagram for describing analysis of changes in cell phenotypes in the macrophage cell line RAW264.7 induced with LPS by humoral factors derived from the mesenchymal stem cells in Examples 3-1 to 3-3.

FIG. 46 shows micrographs of histopathological images of large intestines acquired after transplanting the mesenchymal stem cells in Examples 3-1 to 3-3 and Reference Examples 3-1 and 3-2.

FIG. 47 shows graphs of the expression levels of inflammatory cytokines in intestinal tissues acquired after transplanting the mesenchymal stem cells in Examples 3-1 to 3-3 and Reference Examples 3-1 and 3-2.

FIG. 48 shows micrographs of the surroundings of hydrogel structures in Examples 3-1 to 3-3 and Reference Example 3-1 that have been resected from peritoneal cavities after the transplantation of the hydrogel structures.

FIG. 49 shows graphs of the results of measuring various expression factors related to the mRNA of the mesenchymal stem cells encapsulated in the hydrogel fibers in Examples 4-1 and 4-2.

FIG. 50 shows graphs of the results of measuring various expression factors related to the mRNA of mesenchymal stem cells encapsulated in hydrogels in Examples 5-1 and 5-2.

FIG. 51 shows a graph of the results of measuring a humoral factor (TGF-β1) secreted from the mesenchymal stem cells encapsulated in the hydrogels in Examples 5-1 and 5-2.

FIG. 52 shows a graph of the results of measuring a humoral factor (prostaglandin E2) secreted from the mesenchymal stem cells encapsulated in the hydrogels in Examples 5-1 and 5-2.

FIG. 53 shows graphs of the results of measuring various expression factors related to the mRNA of mesenchymal stem cells in Examples 6-1 to 6-6.

FIG. 54 shows a graph of the results of measuring a humoral factor (TGF-β1) secreted from the mesenchymal stem cells in Examples 6-1 to 6-6.

FIG. 55 shows a graph of the results of measuring a humoral factor (prostaglandin E2) secreted from the mesenchymal stem cells in Examples 6-1 to 6-6.

FIG. 56 is a diagram showing images of autophagy observed under a transmission electron microscope, which are related to microstructures of the mesenchymal stem cells in Examples 6-1 and 6-4.

FIG. 57 shows magnified photographs of hematoxylin and eosin stained cross-sectional images of the mesenchymal stem cells (spheroids) within hydrogel fibers in Examples 6-1 and 6-4.

FIG. 58 shows magnified photographs of the mesenchymal stem cells (spheroids) within the hydrogel fibers in Examples 6-1 and 6-4.

FIG. 59 shows confocal micrographs of immunofluorescence cell staining showing aspects of the expression of an autophagy-related factor p62 in the mesenchymal stem cells (spheroids) in the hydrogel fibers in Examples 6-1 and 6-4.

FIG. 60 shows confocal micrographs of immunofluorescence cell staining showing aspects of the expression of an autophagy-related factor LC-3 in the mesenchymal stem cells (spheroids) in the hydrogel fibers in Examples 6-1 and 6-4.

FIG. 61 is a photograph showing a hydrogel structure according to Examples 7-1 to 7-3 and 8.

FIG. 62 is an image observed under a phase-contrast microscope obtained by enlarging a part of the hydrogel structure according to Example 7-1.

FIG. 63 shows graphs of the results of measuring various expression factors related to the mRNA of the mesenchymal stem cells in Examples 6-1 to 6-3 and 7-1 to 7-3.

FIG. 64 shows a graph of the results of measuring a humoral factor (TGF-β1) secreted from the mesenchymal stem cells in Examples 6-1 to 6-3 and 7-1 to 7-3.

FIG. 65 shows a graph of the results of measuring a humoral factor (prostaglandin E2) secreted from the mesenchymal stem cells in Examples 6-1 to 6-3 and 7-1 to 7-3.

FIG. 66 is a diagram for describing schedules for treatment by the hydrogel structure in Example 8 using TNBS enteritis model rats.

FIG. 67 shows a graph of body weight changes in the TNBS enteritis model rats that have received the various treatments.

FIG. 68 shows a graph of changes in disease activity indices (DAIs) for the TNBS enteritis model rats that have received the various treatments.

FIG. 69 shows a graph of intestinal wet weights in the TNBS enteritis model rats that have received the various treatments.

FIG. 70 shows a graph of gross appearance scores of external appearances of intestines in peritoneal cavities of the TNBS enteritis model rats that have received the various treatments.

FIG. 71 shows graphs of gross lesion occupancy evaluation in mucosal surfaces (internal appearances) of resected and longitudinally opened intestines of the TNBS enteritis model rats that have received the various treatments.

### DESCRIPTION OF EMBODIMENTS

Hereinafter, embodiments will be described with reference to the drawings. In the drawings below, the same or similar parts are given the same or similar reference numerals.

The inventors of the present application found that a hydrogel fiber containing a hydrogel that encapsulates mesenchymal stem cells can be applied in a variety of uses.

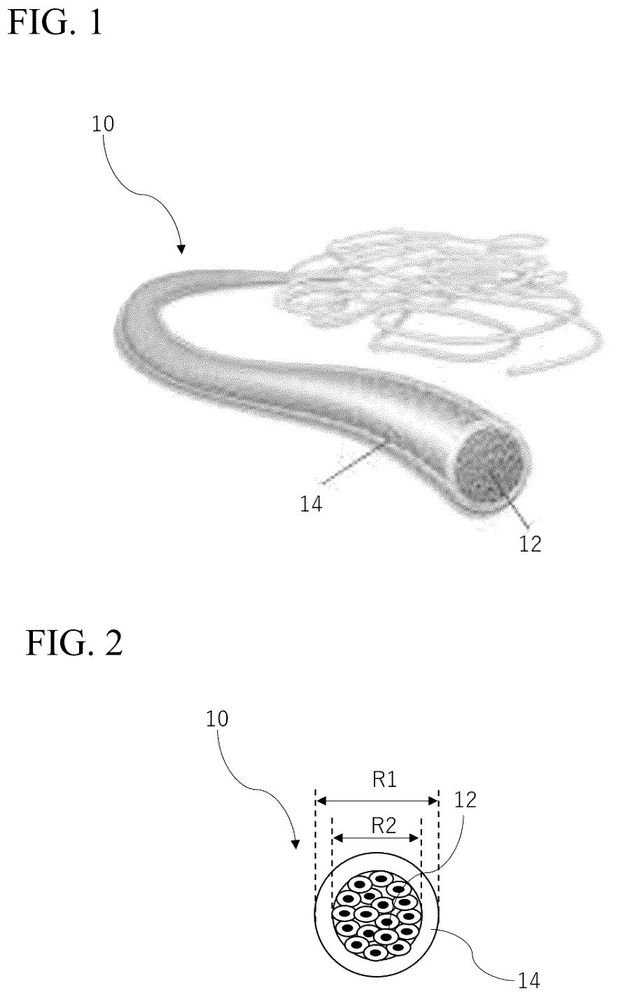

FIG. 1 is a schematic diagram illustrating a structure of a hydrogel fiber according to an embodiment. FIG. 2 is a schematic diagram illustrating a cross-section structure of the hydrogel fiber according to an embodiment.

A hydrogel fiber 10 preferably has a tubular hydrogel 14 and a base material 12 and the mesenchymal stem cells described above provided inside the hydrogel 14.

The base material may contain, for example, a group selected from the group consisting of extracellular matrix, a medium, a chitosan gel, collagen, Matrigel, gelatin, an alginate gel, a peptide gel, laminin, fibronectin, agarose, nanocellulose, methylcellulose, hyaluronic acid, proteoglycan, elastin, pullulan, dextran, pectin, gellan gum, xanthan gum, guar gum, carrageenan, glucomannan, and fibrinogen, or a mixture thereof.

The base material preferably contains extracellular matrix, for example, collagen, laminin, or fibronectin, or a mixture thereof.

The mesenchymal stem cells are not particularly limited, and may be, for example, umbilical cord-derived, placenta-derived, bone marrow-derived, amnion-derived, dental pulp-derived, or adipose-derived mesenchymal stem cells. It is preferable that the mesenchymal stem cells are derived from a human.

The mesenchymal stem cells may be present in the vicinity of the surface of the base material, that is, in the vicinity of the interface between the base material and the hydrogel. Alternatively, the mesenchymal stem cells may be buried within the base material.

The hydrogel is obtained by gelling of a liquid or sol hydrogel precursor. The hydrogel may be a gel containing, for example, an alginate gel as a main component. In this case, the hydrogel precursor may be a solution containing an alginic acid solution as a main component. The hydrogel may contain other materials mixed with the alginate gel.

The alginate gel can be formed by cross-linking the alginic acid solution by a metal ion. The alginic acid solution may be, for example, sodium alginate, potassium alginate, or ammonium alginate, or a combination thereof. The alginic acid solution is easily cross-linked by a metal ion in a short period of time at or near room temperature, thus forming the alginate gel easily. Furthermore, cytotoxicity of the alginate gel is extremely low. Therefore, a hydrogel fiber containing the alginate gel as a main component can be preferably used for various purposes, particularly, for transplantation.

The alginic acid may be a natural extract or chemically modified alginic acid. Examples of the chemically modified alginic acid include methacrylate-modified alginic acid. The hydrogel may also be a mixed system of the above-described alginic acid salt and agar, agarose, polyethylene glycol (PEG), polylactic acid (PLA), nanocellulose, or the like. The weight of the alginic acid salt with respect to the weight of a solvent of the alginic acid solution may be, for example, 0.1 to 10.0 wt %, preferably 0.25 to 7.0 wt %, and more preferably 0.5 to 5.0 wt %.

Examples of the metal ion used for obtaining the alginate gel include a calcium ion, a magnesium ion, a barium ion, a strontium ion, a zinc ion, and an iron ion. The metal ion is preferably a calcium ion or a barium ion.

The metal ion is preferably provided to the alginic acid in a form of a solution. Examples of a solution containing a divalent metal ion include a solution containing a calcium ion. Examples of such a solution include an aqueous solution such as an aqueous calcium chloride solution, an aqueous calcium carbonate solution, and an aqueous calcium gluconate solution. Such a solution is preferably an aqueous calcium chloride solution or an aqueous barium chloride solution.

The type of the alginate gel constituting the hydrogel is preferably a calcium alginate gel or a barium alginate gel.

The base material and/or the hydrogel may contain various growth factors, for example, an epidermal growth factor (EGF), a platelet-derived growth factor (PDGF), a transforming growth factor (TGF), an insulin-like growth factor (IGF), a fibroblast growth factor (FGF), a nerve growth factor (NGF), a vascular endothelial growth factor (VEGF), a hepatocyte growth factor (HGF), or prostaglandin.

The base material and/or the hydrogel may contain various antibiotics as necessary. For example, the base material may contain penicillin-streptomycin as the antibiotics.

The diameter of the hydrogel fiber may be, for example, 100 to 80000 μm, preferably 100 to 5000 μm, and more preferably 200 to 1500 μm. The diameter of the base material in the cross-section of the hydrogel fiber, that is, the inner diameter of the hydrogel may be, for example, 50 to 1000 μm, preferably 80 to 500 μm, and more preferably 100 to 300 μm.

The hydrogel constituting the hydrogel fiber can function as a semipermeable membrane that allows permeation of a component generated by the mesenchymal stem cells and prevents permeation of various cells.

The hydrogel encapsulating the mesenchymal stem cells can be used for, for example, regulating gene expression factors of the mesenchymal stem cells or regulating various secretory components of the mesenchymal stem cells.

Furthermore, the hydrogel encapsulating the mesenchymal stem cells can be used for, for example, transplantation. That is, the hydrogel can be transplanted into a biological body.

The biological body may be an arbitrary animal. The biological body may also be a mammal such as a human, a bovine, a horse, a dog, a cat, or a mouse. Note that the biological body may be an animal other than a human.

Since the mesenchymal stem cells are contained in the hydrogel fiber, the hydrogel fiber can be transplanted directly at the location of a diseased site. Furthermore, since the hydrogel fiber has a fibrous shape, the hydrogel fiber can be extracted from the body as necessary.

In a case where the hydrogel fiber is transplanted into the body, the mesenchymal stem cells in the hydrogel fiber may be autologous cells or allogeneic cells. In a case where the mesenchymal stem cells are autologous cells, the risk of rejection can be further reduced. The risk can also be reduced in a case where the mesenchymal stem cells are allogeneic cells, since the hydrogel prevents the permeation of immune cells.

The hydrogel encapsulating the mesenchymal stem cells can be used for, for example, at least one of suppression of fibrogenesis, suppression of inflammatory cell infiltration, or tissue repair and regeneration. The inflammatory cell infiltration during the transplantation into the body can be suppressed by a synergistic effect between the hydrogel, particularly, the alginate gel, and the mesenchymal stem cells.

In addition, the hydrogel encapsulating the mesenchymal stem cells can be used for, for example, treating enteritis, acute GVHD, small intestinal lesion, hepatitis/hepatic cirrhosis, pancreatitis, or renal dysfunction, or preventing enteritis. In particular, the hydrogel encapsulating the mesenchymal stem cells can be preferably used for treating enteritis.

Preferable examples of the types of enteritis include an inflammatory bowel disease such as ulcerative colitis, Crohn's disease, or intestinal Behcet's disease, drug-induced enterocolitis caused by a drug such as an anticancer agent or an antibiotic, and radiation enteritis caused by radiation.

Furthermore, the hydrogel encapsulating the mesenchymal stem cells can be used for extracting a supernatant of a mesenchymal stem cell culture. The mesenchymal stem cells encapsulated in the hydrogel are immersed in a culture medium in a state of being encapsulated in the hydrogel. In this manner, the mesenchymal stem cells can be cultured in the hydrogel.

The supernatant of the culture medium in which the mesenchymal stem cells are cultured in a state of being encapsulated in the hydrogel fiber can be used as, for example, an agent for treating enteritis or preventing enteritis. The agent for treating enteritis or preventing enteritis may contain the supernatant of the culture medium in which the mesenchymal stem cells are cultured in a state of being encapsulated in the hydrogel fiber as a main component or may only contain the supernatant of the culture medium. Here, since the mesenchymal stem cells are in a state of being encapsulated in the hydrogel fiber, the extraction of the supernatant of the culture medium can be easily performed.

(Method for Producing Hydrogel Fiber)

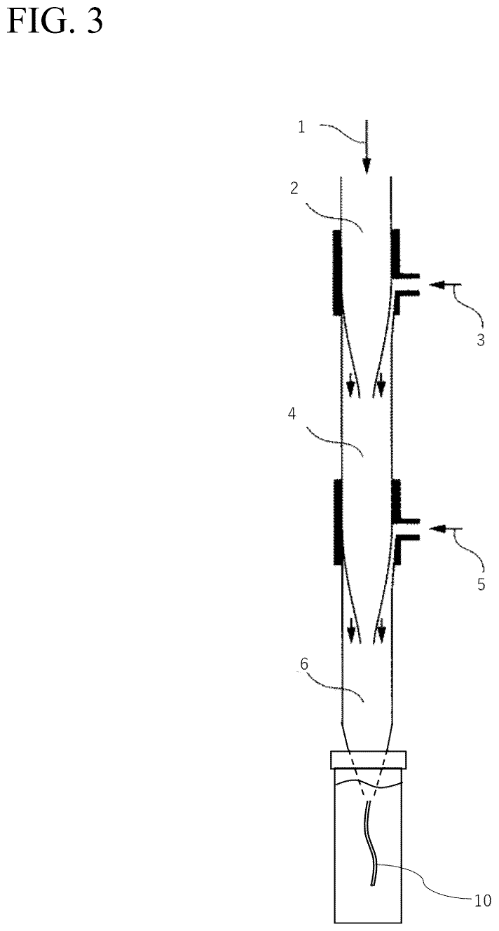

FIG. 3 is a schematic diagram illustrating an example of an apparatus for producing the above-described hydrogel fiber.

First, a first laminar flow of a cell suspension 1 containing cells and a base material is formed. The first laminar flow is formed within a first introduction pipe 2. Here, the detailed description of the base material and the cells are as described above.

A second laminar flow of a hydrogel preparation solution 3 that covers the outer perimeter of the first laminar flow is also formed. As a result, the hydrogel preparation solution (second laminar flow) 3 surrounding the flow of the cell suspension 1 (first laminar flow) is formed in a second introduction pipe 4. Here, the hydrogel preparation solution may be any liquid or sol that forms a hydrogel by gelling.

In addition, a gelling material that causes the gelling of the hydrogel preparation solution is applied on the outer perimeter of the hydrogel preparation solution (second laminar flow) 3. In the aspect shown in FIG. 3, a third laminar flow of a solution 5 which serves as the gelling material is formed. The solution 5 surrounds the hydrogel preparation solution (second laminar flow) 3 in a third introduction pipe 6.

The first laminar flow, the second laminar flow, and the third laminar flow exit from the third introduction pipe 6 and are plunged into a liquid such as saline. Here, the hydrogel preparation solution exits from the third introduction pipe 6 while becoming a gel by the application of the gelling material. As a result, the hydrogel fiber described above is formed in the liquid such as saline.

After the hydrogel fiber 10 is formed, the hydrogel fiber 10 may be immersed in a medium such as a liquid medium as necessary. The mesenchymal stem cells may be cultured and grown within the hydrogel fiber 10 in this manner.

In the above-described aspect, the hydrogel fiber was formed by forming the first laminar flow, the second laminar flow, and the third laminar flow, and allowing the flows to exit from the third introduction pipe 6. Alternatively, the hydrogel fiber can also be produced by forming a first laminar flow of a cell suspension that contains cells and a base material, forming a second laminar flow of a hydrogel preparation solution that covers the outer perimeter of the first laminar flow, and then discharging the first laminar flow and the second laminar flow into a container containing a solution serving as a gelling material.

Note that the hydrogel fiber described above can also be prepared by, for example, the methods described in WO 2011/046105 A and WO 2015/178427 A.

In the above-described aspect, the shape of the hydrogel constituting the hydrogel structure was a fiber shape such as a tubular shape or a string shape. Alternatively, the shape of the hydrogel constituting the hydrogel structure is not particularly limited. The inventors found that, in this case as well, the hydrogel structure containing the hydrogel that encapsulates the mesenchymal stem cells can be applied in a variety of uses. In other words, a hydrogel structure containing a base material that contains mesenchymal stem cells and a hydrogel that encapsulates the base material can be applied in a variety of novel uses. For example, the hydrogel that encapsulates the base material may have a shape such as a spherical shape or a spherical shell shape. Here, the materials forming the base material and the hydrogel are as described above.

Furthermore, the hydrogel structure may contain a form shaped with the hydrogel that encapsulates the mesenchymal stem cells and has the above-described shape, and a second hydrogel that encapsulates the shaped form.

The form shaped with the hydrogel that encapsulates the mesenchymal stem cells and has the above-described shape may contain a fibrous hydrogel (hydrogel fiber) which is shaped in a regular manner. For example, the form contains a hydrogel fiber formed into a spiral shape, a grid shape, a lattice shape, and/or a mesh shape. A hydrogel fiber having a spiral shape may be formed by, for example, a fibrous hydrogel wound around a support. In addition, a hydrogel fiber having a sheet shape may be formed by, for example, a meandering hydrogel fiber that is formed on a support having a sheet shape. The fibrous hydrogel which is shaped in a regular manner may or may not be attached to the support. The fibrous hydrogel which is shaped in a regular manner may be shaped in a state of being attached to the support and then detached from the support.

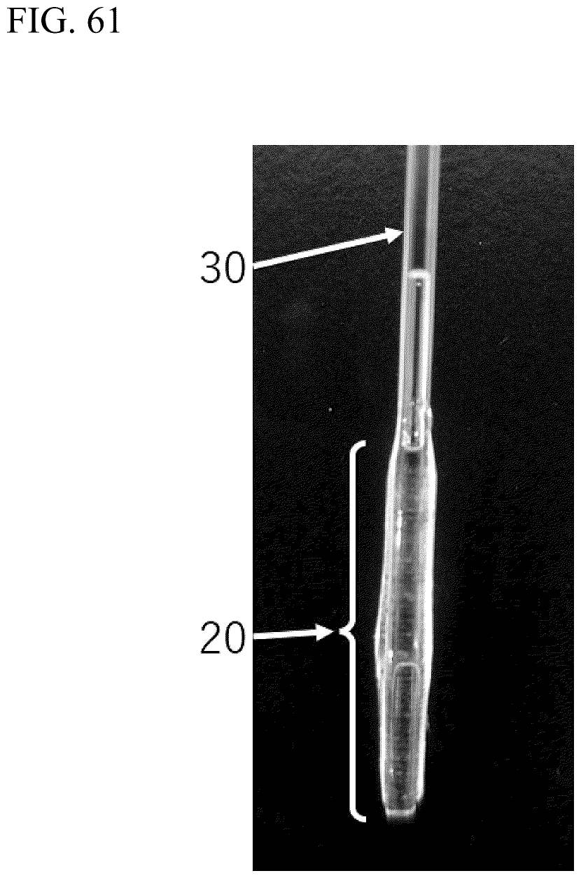

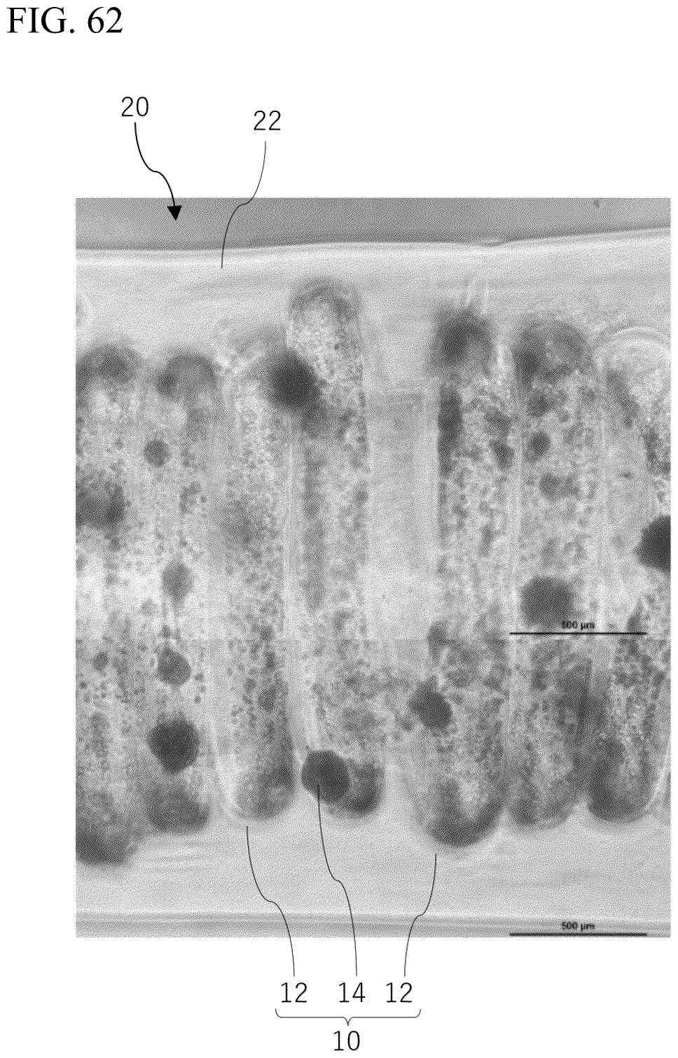

A hydrogel structure 20 containing a fibrous hydrogel that is spirally wound is formed by winding the above-described hydrogel fiber 10 around, for example, a long support such as a glass rod 30, and then covering the wound hydrogel fiber with a second hydrogel 22 (also refer to FIGS. 61 and 62). In this case, the hydrogel structure 20 may be maintained in a state of being attached to the support or detached from the support.

The second hydrogel described above may be formed so as to entirely cover the fibrous hydrogel (hydrogel fiber) that is wound around the long support. In this case, there is an advantage in that the second hydrogel is easily formed. Alternatively, the second hydrogel described above may be formed so as to cover an exposed portion of the fibrous hydrogel (hydrogel fiber) that is wound around the long support along the hydrogel fiber.

A hydrogel structure containing a fibrous hydrogel that is shaped into a sheet shape is formed by the above-described hydrogel fiber into a sheet shape and then covering the hydrogel fiber with the second hydrogel. A support having a sheet shape can be shaped, for example, on a support having a sheet shape. In this case, the second hydrogel described above may be formed so as to cover the hydrogel fiber that is shaped on the support having a sheet shape.

The second hydrogel is obtained by gelling of a liquid or sol hydrogel precursor. The second hydrogel may be a gel containing, for example, an alginate gel as a main component. In this case, the hydrogel precursor may be a solution containing an alginic acid solution as a main component. The second hydrogel may contain other materials mixed with the alginate gel.

The alginate gel can be formed by cross-linking the alginic acid solution by a metal ion. The alginic acid solution may be, for example, sodium alginate, potassium alginate, or ammonium alginate, or a combination thereof. The alginic acid may be a natural extract or chemically modified alginic acid. Examples of the chemically modified alginic acid include methacrylate-modified alginic acid.

The second hydrogel may also be a mixed system of the above-described alginic acid salt and agar, agarose, polyethylene glycol (PEG), polylactic acid (PLA), nanocellulose, or the like.

The hydrogel structure containing the form shaped by the hydrogel that encapsulates the mesenchymal stem cells and has the above-described shape and the second hydrogel that encapsulates the form can be applied in uses such as transplantation or a topical agent for medical care. Such a hydrogel structure can be used for, for example, application to an internal organ, a mucous membrane, and/or skin. Therefore, the hydrogel structure may have a shape that is suitable for the application to an internal organ, a mucous membrane, and/or skin.

In a specific example, the hydrogel structure containing the fibrous hydrogel that has a sheet shape or is spirally wound may be configured, for example, to contact the internal organ, mucous membrane, and/or skin, and preferably cover a surface in the vicinity of the diseased site. The hydrogel structure containing the fibrous hydrogel that is spirally wound may be configured, for example, to be insertable into a fistula in an anal fistula.

The method of inserting the hydrogel structure into a fistula in an anal fistula can be used as an improved Kshara Sutra anal fistula treatment. In the Kshara Sutra anal fistula treatment, a thick kite string-like thread “Kshara Sutra” is impregnated with 3 types of plant-derived drugs and inserted into a fistula in order to open and ultimately cure the fistula. The thread is changed once a week. Although the treatment duration is long, the curing progresses with a new granulation tissue while dissolving the tissue of the fistula at the same time. The hydrogel structure according to the above aspect, which encapsulates the mesenchymal stem cells, can be used in place of this thread.

In addition to the above-described use for transplantation, the hydrogel structure of the above aspect can also be applied as a topical agent. Therefore, the hydrogel structure may not only be transplanted into the body but also applied to skin or a mucous membrane. As used herein, the term “topical agent” includes, for example, agents applied to a mucous membrane such as a hemorrhoid or an intestinal tract.

The hydrogel structure and the culture supernatant extracted from the culture medium in which the mesenchymal stem cells are cultured together with the hydrogel structure can also be used for extraction, enhancement, and suppression of various factors, in addition to the uses for the treatment and the prevention described above.

For example, the hydrogel structure can be used as an enhancing agent for the expression of a hypoxia-responsive factor, and/or as an enhancing agent for the expression of an antioxidant stress-related factor, and/or a tissue repair-related factor, and/or an immunoregulatory factor, and/or a tumor suppressor gene/cell senescence-related factor in the mesenchymal stem cells.

In addition, the hydrogel structure or the culture supernatant extracted from the culture medium in which the mesenchymal stem cells are cultured together with the hydrogel structure can be used as, for example, a macrophage activity-modifying agent.

Moreover, the hydrogel structure or the culture supernatant extracted from the culture medium in which the mesenchymal stem cells are cultured together with the hydrogel structure can be used as, for example, a protecting agent against cellular damage in epithelial cells and/or an apoptosis regulating agent.

Furthermore, the mesenchymal stem cells contained in the above-described hydrogel structure may form spheroids. When the mesenchymal stem cells are cultured in a state of being encapsulated in the hydrogel, spheroids are easily formed in the culture process. Specifically, in a case where a storage modulus (G′) of the hydrogel fiber at a frequency of 1 Hz is, for example, 100 Pa or more, preferably 180 Pa or more, and more preferably 400 Pa or more, the mesenchymal stem cells in the hydrogel fiber easily form the spheroids in the culture process. Here, the value of the storage modulus (G′) may be a value measured at a temperature of 28° C. In addition, instead of being disorderly formed, the variation in the forms of the spheroids (shapes and sizes) tends to be small, as the spheroids are confined to the form of the inner cavity of the hydrogel. Thus, it is assumed that the mesenchymal stem cells contained in the hydrogel structure can form the spheroids in a state of maintaining the differentiation potential. It is preferable that the mesenchymal stem cells form the spheroids in a state of maintaining the pluripotent differentiation potential or versatile differentiation potential.

More specifically, the spheroid in the hydrogel structure may have a central portion formed by degenerated mesenchymal stem cells and multiple layers, for example, double- or triple-layer, of viable cells present around the central portion. The spheroid may also contain extracellular matrix (for example, type I collagen, fibronectin, or laminin) that is obtained by degeneration of the mesenchymal stem cells or secretion from the mesenchymal stem cells, or is encapsulated along with the cells. In this case, the hydrogel and the extracellular matrix may be unevenly distributed within the spheroid.

The present inventors found that the mesenchymal stem cells that are in the state of being encapsulated in the hydrogel can survive for a long period of time and secrete various functional factors for a long period of time. Although hypothetical, this is considered to be realized by the confinement of the spheroids to the form of the inner cavity of the hydrogel by the encapsulation thereof in the hydrogel, which allows the variation in the forms of the spheroids (shapes and sizes) to be small, whereby the activation of autophagy, enhancement of the hypoxia-responsive factor expression, an antioxidant stress stress mechanism, and/or an immunoregulatory mechanism is promoted. From this point of view, the storage modulus (G′) of the hydrogel fiber at a frequency of 1 Hz may be, for example, 100 Pa or more, preferably 180 Pa or more, and more preferably 400 Pa or more.

### EXAMPLES

Next, Examples will be described in detail.

[Production of Hydrogel Fiber]

First, a core solution, a hydrogel preparation solution, and a gelling material were prepared. In Examples 1-1, 1-2, 2-1 to 2-4, 3-1 to 3-3, 4-1, and 4-2 and Reference Examples 2-2, 2-3, and 3-1 shown in Table 1 below, the hydrogel preparation solutions are a sodium alginate solution. The sodium alginate solution is a solution obtained by mixing a sodium alginate “KIMICA ALGIN High G-series “I-3G”” manufactured by KIMICA Corporation with saline. Here, the concentration of the sodium alginate with respect to the saline was 1.44 wt %. Note that wt % is defined by the weight (g) of a solute, in this case, the sodium alginate, in an aqueous solution per 100 g of a solvent.

In the following Examples 2-2 and 2-4 and Reference Example 2-3, an aqueous barium chloride solution was used as the gelling material. In Examples 1-1, 1-2, 2-1, 2-3, 3-1, 3-2, 3-3, 4-1, and 4-2 and Reference Examples 2-2 and 3-1, an aqueous calcium chloride solution was used as the gelling material. Therefore, hydrogels forming hydrogel fibers produced in Examples and Reference Examples in which the aqueous barium chloride solution was used are formed by a barium alginate gel. On the other hand, hydrogels forming hydrogel fibers produced in Examples and Reference Examples in which the aqueous calcium chloride solution was used are formed by a calcium alginate gel.

The core solution is a solution for suspending cells. The core solution (base material) differs for each Example and Reference Example. The core solution prepared for each Example and Reference Example is described below.

TABLE 1

| | | Cells | Base material | Hydrogel | Application example |
|---|---|---|---|---|---|
| Example 1-1 | | MSC | with native collagen | Calcium alginate | TNBS induced |
| | | | | | enteritis |
| Example 1-2 | | MSC | Medium | Calcium alginate | — |
| Reference example 1-1 | | *MSC (2D culture)/direct transplantation | | | TNBS induced |
| | | | | | enteritis |
| Reference example 1-2 | | *Administration of medium only | | | TNBS induced |
| | | | | | enteritis |
| Reference example 1-3 | | — | — | — | Normal control |
| Example 2-1 | Example | MSC | with native collagen | Calcium alginate | Enteritis transfected |
| Example 2-2 | 2-A | MSC | with native collagen | Barium alginate | with naive T cells |
| Example 2-3 | | MSC | Medium | Calcium alginate | — |
| Example 2-4 | Example | MSC | Medium | Barium alginate | Enteritis transfected |
| | 2-B | | | | with naive T cells |
| Reference | | *MSC (2D culture)/direct transplantation | | | Enteritis transfected |
| example 2-1 | | | | | with naive T cells |
| Reference | Reference | — | with native collagen | Calcium alginate | Enteritis transfected |
| example 2-2 | example | | | | with naive T cells |
| Reference | 2-A | — | with native collagen | Barium alginate | Enteritis transfected |
| example 2-3 | | | | | with naive T cells |
| Reference | | *Administration of medium only | | | Enteritis transfected |
| example 2-4 | | | | | with naive T cells |
| Reference | | — | — | — | Normal control |
| example 2-5 | | | | | |

| Example 3-1 | MSC | with atelocollagen | Calcium alginate | Enteritis transfected |
|---|---|---|---|---|
| | | | | with naive T cells |
| Example 3-2 | MSC | with fibronectin | Calcium alginate | Enteritis transfected |
| | | | | with naive T cells |
| Example 3-3 | MSC | with laminin | Calcium alginate | Enteritis transfected |
| | | | | with naive T cells |
| Reference | — | Medium | Calcium alginate | Enteritis transfected |
| example 3-1 | | | | with naive T cells |
| Reference | — | — | — | Normal control |
| example 3-2 | | | | |
| Example 4-1 | MSC | with native collagen | Calcium alginate | DSS enteritis |
| Example 4-2 | MSC | Medium | Calcium alginate | — |
| Reference | *Administration of medium only | | | DSS enteritis |
| example 4-1 | | | | |

The core solutions in Examples 1-1, 2-1, 2-2, and 4-1 and Reference Examples 2-2 and 2-3 are a native collagen solution. A solution obtained by adding buffer to a 5 mg/mL collagen acidic solution I-AC so that the solution becomes neutral was used as the collagen solution. The final concentration of the collagen acidic solution I-AC is 4 mg/mL.

The core solutions in Examples 1-2, 2-3, 2-4, and 4-2 and Reference Example 3-1 are a medium. The medium is obtained by adding fetal bovine serum (FBS) and an antibiotic to a GlutaMAX medium (MEM α, nucleosides, GlutaMAX™) (manufactured by Thermo Fisher Scientific Inc.: Cat No. 32571-036). The GlutaMAX medium is obtained by adding GlutaMAX supplement to αMEM. The mixing was performed so that the ratio between the GlutaMAX medium, FBS, and the antibiotic was 89:10:1 at a temperature of 37° C. in terms of a volume ratio. Note that the core solutions in Examples 1-2, 2-3, 2-4, and 4-2 and Reference Example 3-1 do not contain additional extracellular matrix.

The core solution in Example 3-1 is an atelocollagen solution. A 5 mg/mL collagen acidic solution I-PC was used as the collagen solution.

The core solution in Example 3-2 is a fibronectin solution. The fibronectin solution is obtained by dissolving human plasma-derived fibronectin (Corning Incorporated; Product Number 354008) in phosphate-buffered saline (PBS).

The core solution in Example 3-3 is a laminin solution (manufactured by VERITAS Corporation; Human recombinant laminin 511).

In each Example, the cells suspended in the core solution are human umbilical cord-derived mesenchymal stem cells. In Examples 1-1, 1-2, 2-1 to 2-4, 3-1 to 3-3, 4-1, and 4-2, the density of the cells contained in the cell suspension was about 1×10⁸ cells/mL.

In Reference Examples 2-2, 2-3, and 3-1, cells were not suspended in the core solutions. In other words, the hydrogel fibers produced in Reference Examples 2-2, 2-3, and 3-1 do not contain cells.

Hydrogel fibers were prepared using the core solution, the hydrogel preparation solution, and the gelling material described above according to the method for producing a hydrogel fiber described above. That is, a first laminar flow of the core solution, a second laminar flow of the sodium alginate solution, and a third laminar flow of the aqueous calcium chloride solution or aqueous barium chloride solution were formed, the second laminar flow and the third laminar flow being formed around the first laminar flow and the second laminar flow, respectively, and these laminar flows were discharged into saline. As a result, elongated hydrogel fibers were produced in the saline.

Here, the volume of the cell suspension (core solution) encapsulated in the hydrogel fiber in each Example was about 10 μL. Therefore, when preparing the hydrogel fiber in each Example, the number of the cells encapsulated in one hydrogel fiber was about 10⁶ cells.

The cross-sectional diameters of the hydrogel fibers thus produced were 200 to 400 μm, and the inner diameters were about 50 to 300 μm. The lengths of the hydrogel fibers were about 25 cm. However, it should be noted that the lengths of the hydrogel fibers are not particularly limited.

As a result of the above procedures, the hydrogel fiber encapsulating the mesenchymal stem cells was produced in each Example. The hydrogel fiber may be transferred into a liquid medium to culture the mesenchymal stem cells in the hydrogel fiber as necessary.

Note that, as described above, the hydrogel fibers produced in Reference Examples 2-2, 2-3, and 3-1 do not contain cells. In addition, Reference Examples 1-1, 1-2, 1-3, 2-1, 2-4, 2-5, 3-2, and 4-1 in Table 1 are examples used in the transplantation experiments described later, and the details thereof will be described later.

[Hydrogel Fiber Characteristic Analysis (1)]

### Examples 1-1 and 1-2

The cases of immersing and culturing the mesenchymal stem cells encapsulated in the hydrogel fibers in Examples 1-1 and 1-2 in the GlutaMAX medium containing FBS and the antibiotic together with the fibers were compared with the case of performing 2-dimensional culture of the mesenchymal stem cells without encapsulating the cells in the hydrogel fibers (Reference Example 1-1). Specifically, the amount of various humoral factors secreted into the medium and various expression factors related to mRNA were measured.

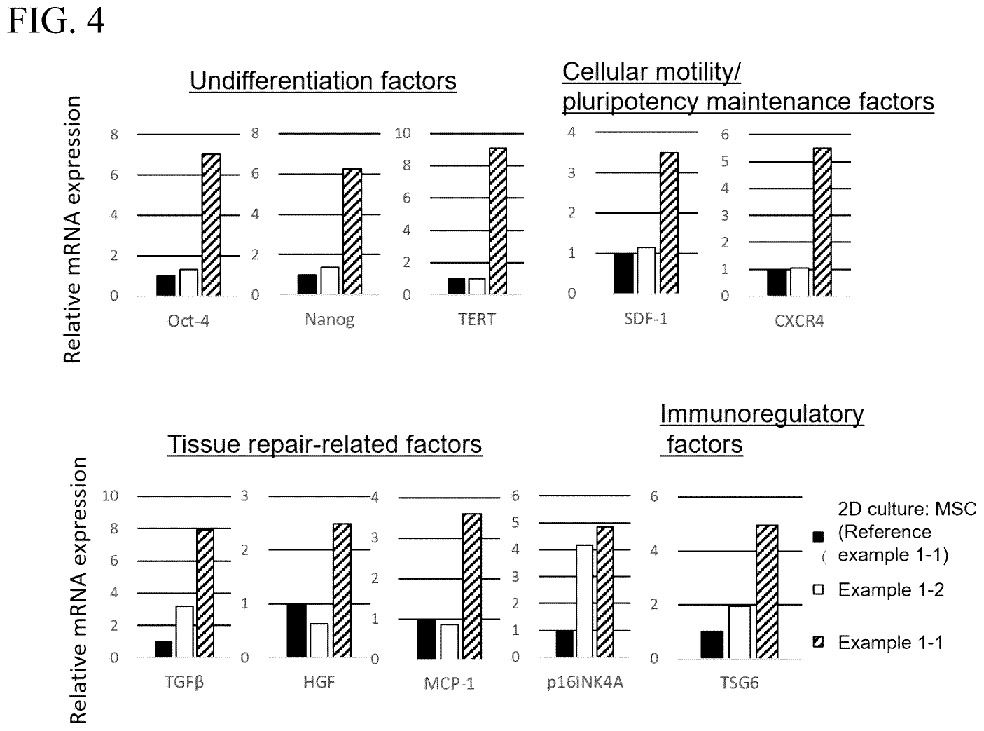

FIG. 4 shows graphs of the results of measuring various expression factors related to the mRNA of the mesenchymal stem cells subjected to the 2-dimensional culture (Reference Example 1-1) and the mesenchymal stem cells encapsulated in the hydrogel fibers in Examples 1-1 and 1-2. The vertical axes represent the ratios obtained when the value for the mesenchymal stem cells subjected to the 2-dimensional culture (Reference Example 1-1) was normalized as “1”. Note that, in FIG. 4, Reference Example 1-1 indicates the results obtained by collecting the cells after culturing the cells for 72 hours and performing the measurement, and each Example indicates the results obtained by performing the measurement 18 days after the preparation of the hydrogel fiber.

FIG. 4 shows undifferentiation factors (Oct-4, Nanog, and TERT), cellular motility/pluripotency maintenance factors (SDF-1 and CXCR4), tissue repair and regeneration-related factors (TGFβ, HGF, and MCP-1), a cell senescence-related factor and tumor suppressor gene (p16INK4A), and an immunoregulatory factor (TSG6). TGFβ, HGF, and MCP-1 are factors that contribute to the repair and the regeneration of a tissue that has been damaged by inflammation or the like.

The expression levels of all factors in the mesenchymal stem cells encapsulated in the hydrogel fibers in Examples 1-1 and 1-2 were equal to or higher than the expression levels of all factors in the 2-dimensional culture (Reference Example 1-1). It is thus found that the encapsulation of the mesenchymal stem cells in microfibers can contribute to the increase of a number of expression factors related to the mRNA.

Furthermore, the expression levels of the above expression factors in Example 1-1 were higher than those in Example 1-2. It is thus understood that contribution to the expression levels of the expression factors is greater when the microfibers contain the extracellular matrix (scaffold), which is collagen in the present Example 1-1.

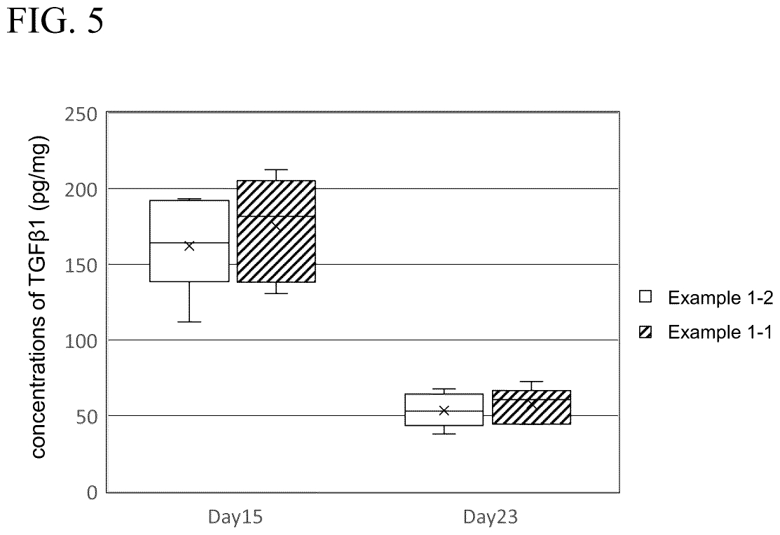

FIG. 5 shows a graph of the results of measuring a tissue-repair factor (TGF-β1) derived from the mesenchymal stem cells encapsulated in the hydrogel fibers in Examples 1-1 and 1-2. The vertical axis in FIG. 5 represents the concentration of TGF-β1 in the medium. The horizontal axis in FIG. 5 represents the number of days that have passed since the preparation of the hydrogel fibers described above (culture period). When the day on which the hydrogel fibers were prepared was set to Day 0, TGF-β1 was measured on Day 15 and Day 23. In FIG. 5, the rectangles with oblique lines represent the experimental results obtained with the hydrogel fiber in Example 1-1. The blank rectangles represent the experimental results obtained with the hydrogel fiber in Example 1-2. In each of Examples 1-1 and 1-2, the experiment was conducted with 3 hydrogel fibers. The central value in the longitudinal direction in each rectangle is the mean value of the results of the experiment conducted with the 3 hydrogel fibers. The length of each rectangle in the longitudinal direction represents the standard deviation (dispersion) of the results of the experiment conducted with the 3 hydrogel fibers.

The amounts of TGF-β1 secreted were nearly equal in both Examples 1-1 and 1-2.

In both Examples 1-1 and 1-2, the amounts of TGF-β1 secreted decreased as the number of days (culture period) increased since the preparation of the hydrogel fibers.

[Hydrogel Fiber Characteristic Analysis (2)]

### Examples 2-1 to 2-4

Measurement of a paracrine factor (TGF-β1) derived from the mesenchymal stem cells in Examples 2-1 to 2-4 and measurement of various expression factors related to the mRNA were measured.

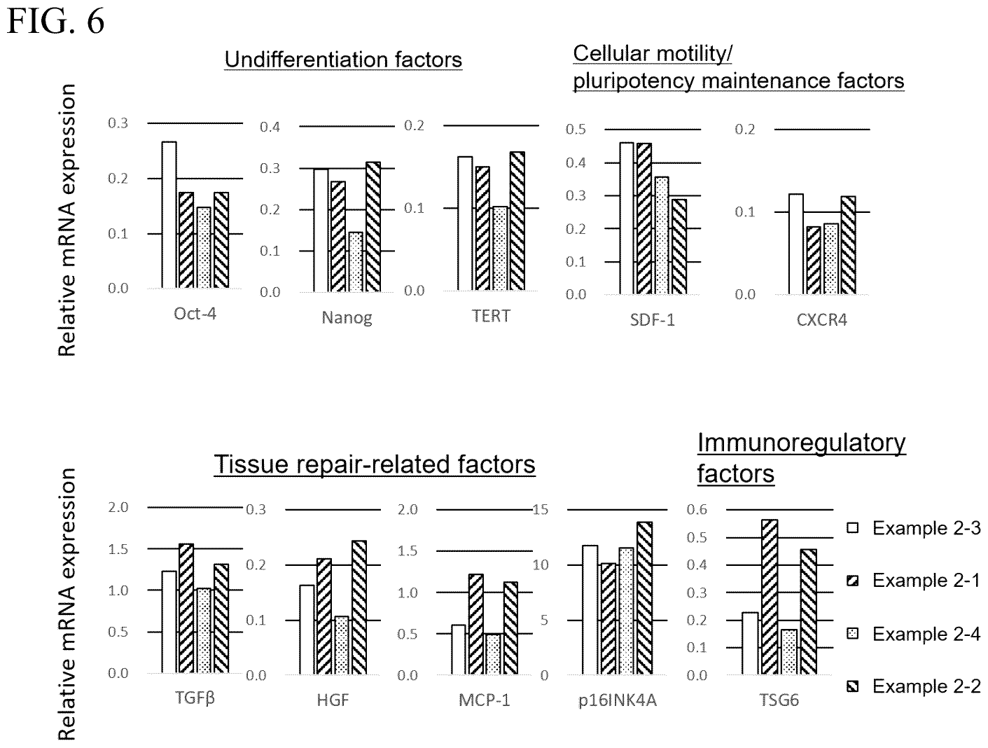

FIG. 6 shows graphs of the results of measuring various expression factors related to the mRNA of the mesenchymal stem cells encapsulated in the hydrogel fibers in Examples 2-1 to 2-4. Specifically, when the day on which culture was started was set to Day 0, the various expression factors related to the mRNA were measured on Day 30.

FIG. 6 shows undifferentiation factors (Oct-4, Nanog, and TERT), cellular motility/pluripotency maintenance factors (SDF-1 and CXCR4), tissue repair-related factors (TGFβ and MCP-1), a cell senescence-related factor and tumor suppressor gene (p16INK4A), and an immunoregulatory factor (TSG6). TGET, HGF, and MCP-1 are factors that contribute to the repair and the regeneration of a tissue that has been damaged by inflammation or the like.

Regardless of the type of the hydrogel, the expression levels of the tissue repair and regeneration-related factors (TGFβ, HGF, and MCP-1) and the immunoregulatory factor (TSG6) were higher in the hydrogel fibers containing collagen (Examples 2-1 and 2-2) than in the hydrogel fibers not containing collagen (Examples 2-3 and 2-4).

In a case where the hydrogel was barium alginate, the expression levels of the undifferentiation factors (Nanog and TERT) were also higher in the hydrogel fibers containing collagen (Example 2-2) than in the hydrogel fibers not containing collagen (Example 2-4).

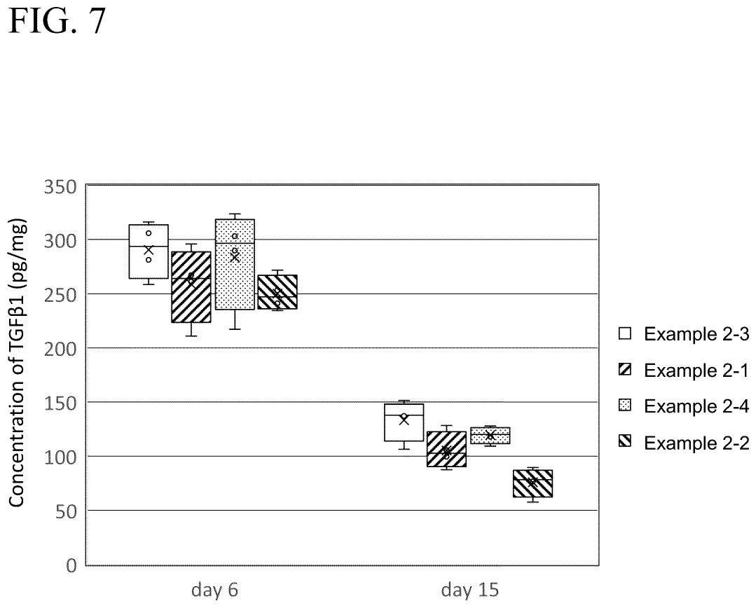

FIG. 7 shows a graph of the results of measuring a tissue-repair factor (TGF-β1) of the mesenchymal stem cells encapsulated in the hydrogel fibers in Examples 2-1 to 2-4. The vertical axis in FIG. 7 represents the amount of TGF-β1 per 1 mg of the total proteins in the medium. More specifically, the vertical axis represents the value obtained after correcting the amount of TGF-β1 by the protein concentration. When the day on which the hydrogel fibers were prepared was set to Day 0, TGF-β1 was measured on Day 6 and Day 15. The central value in the longitudinal direction in each rectangle in FIG. 7 is the mean value of the results of the experiment conducted with a plurality of hydrogel fibers. The length of each rectangle in the longitudinal direction represents the standard deviation (dispersion) of the results of the experiment conducted with the plurality of hydrogel fibers.

As shown in FIG. 7, the amounts of TGF-β1 secreted tended to be slightly greater in the hydrogel fibers not containing collagen (Examples 2-3 and 2-4) than in the hydrogel fibers containing collagen as the extracellular matrix (Examples 2-1 and 2-2). Therefore, as for this lot of mesenchymal stem cells, the hydrogel fibers not containing collagen can be preferably used for the purpose of secreting TGF-β1.

[Hydrogel Fiber Characteristic Analysis (3)]

### Examples 3-1 to 3-3

Measurement of a paracrine factor (TGF-β1) derived from the mesenchymal stem cells in Examples 3-1 to 3-3 and measurement of various expression factors related to the mRNA were measured.

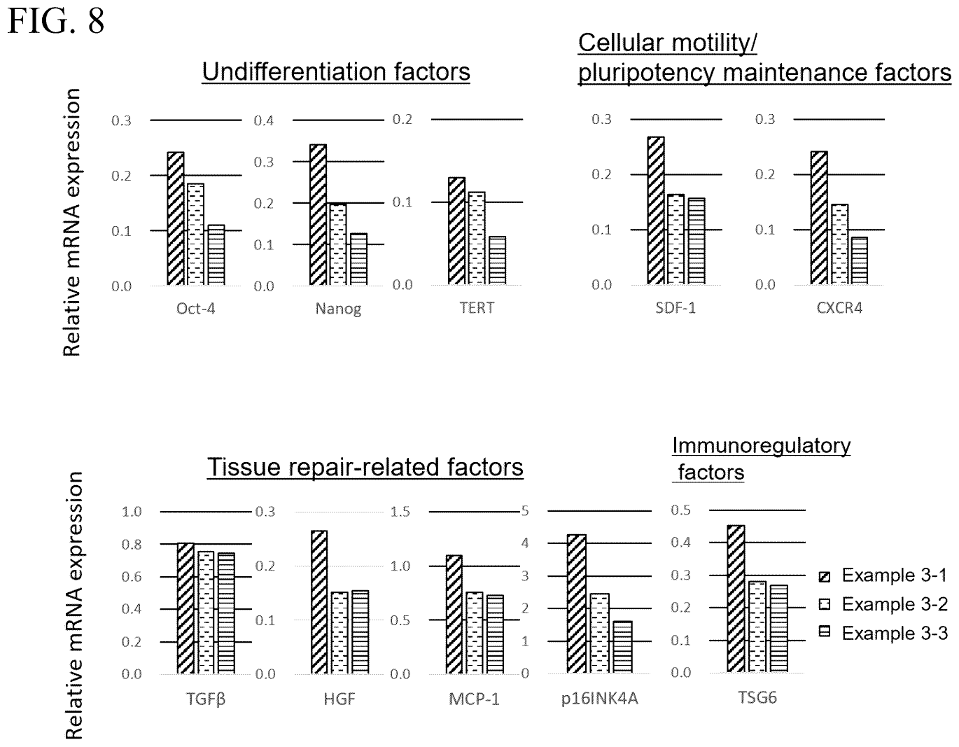

FIG. 8 shows graphs of the results of measuring various expression factors related to the mRNA of the mesenchymal stem cells encapsulated in the hydrogel fibers in Examples 3-1 to 3-3. Specifically, when the day on which culture was started was set to Day 0, the various expression factors related to the mRNA were measured on Day 9.

FIG. 8 shows undifferentiation factors (Oct-4, Nanog, and TERT), cellular motility/pluripotency maintenance factors (SDF-1 and CXCR4), tissue repair-related factors (TGFβ, HGF, and MCP-1), a cell senescence-related factor and tumor suppressor gene (p16INK4A), and an immunoregulatory factor (TSG6). TGFβ, HGF, and MCP-1 are factors that contribute to the repair and the regeneration of a tissue that has been damaged by inflammation or the like.

FIG. 8 shows the expression level of each factor in each Example obtained when an appropriate reference value was normalized as “1”.

Referring to FIG. 8, the expression levels of almost all factors were relatively high in the hydrogel fibers containing atelocollagen (Example 3-1). Next, the expression levels were high in the hydrogel fibers containing fibronectin (Example 3-2), and the expression levels in the hydrogel fibers containing laminin (Example 3-3) were equal to or lower than those in the hydrogel fibers containing fibronectin.

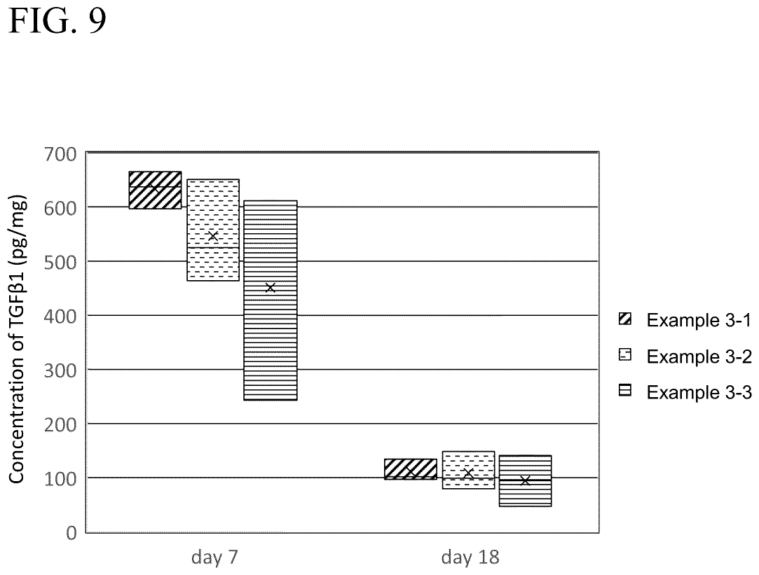

FIG. 9 shows a graph of the results of measuring a tissue-repair factor (TGF-β1) derived from the mesenchymal stem cells encapsulated in the hydrogel fibers in Examples 3-1 and 3-3. The vertical axis in FIG. 9 represents the amount of TGF-β1 per 1 mg of the total proteins in the medium. More specifically, the vertical axis represents the value obtained after correcting the amount of TGF-β1 by the protein concentration. When the day on which culture of the cells was started was set to Day 0, TGF-β1 was measured on Day 7 and Day 18. The central value in the longitudinal direction in each rectangle in FIG. 9 is the mean value of the results of the experiment conducted with a plurality of hydrogel fibers. The length of each rectangle in the longitudinal direction represents the standard deviation (dispersion) of the results of the experiment conducted with the plurality of hydrogel fibers.

As shown in FIG. 9, the amounts of TGF-β1 secreted were relatively great in a case where the hydrogel fibers contained atelocollagen (Example 3-1) and in a case where the hydrogel fibers contained fibronectin (Example 3-2), especially on Day 7 when the culturing days were short since the hydrogel fiber preparation. On Day 18 after the fiber preparation, no difference was observed among Examples 3-1 to 3-3.

In all of Examples 3-1 to 3-3, the amounts of TGF-β1 secreted decreased as the number of days (culture period) increased since the preparation of the hydrogel fibers.

The hydrogel fibers containing atelocollagen or fibronectin can be one of the promising candidates for the factor expression and uses related therewith.

[Hydrogel Fiber Characteristic Analysis (4)]

### Examples 4-1 and 4-2

In Examples 4-1 and 4-2, measurement of paracrine factors (TGF-β1, VEGF, and PGE2) derived from the mesenchymal stem cells and various expression factors related to the mRNA were measured.

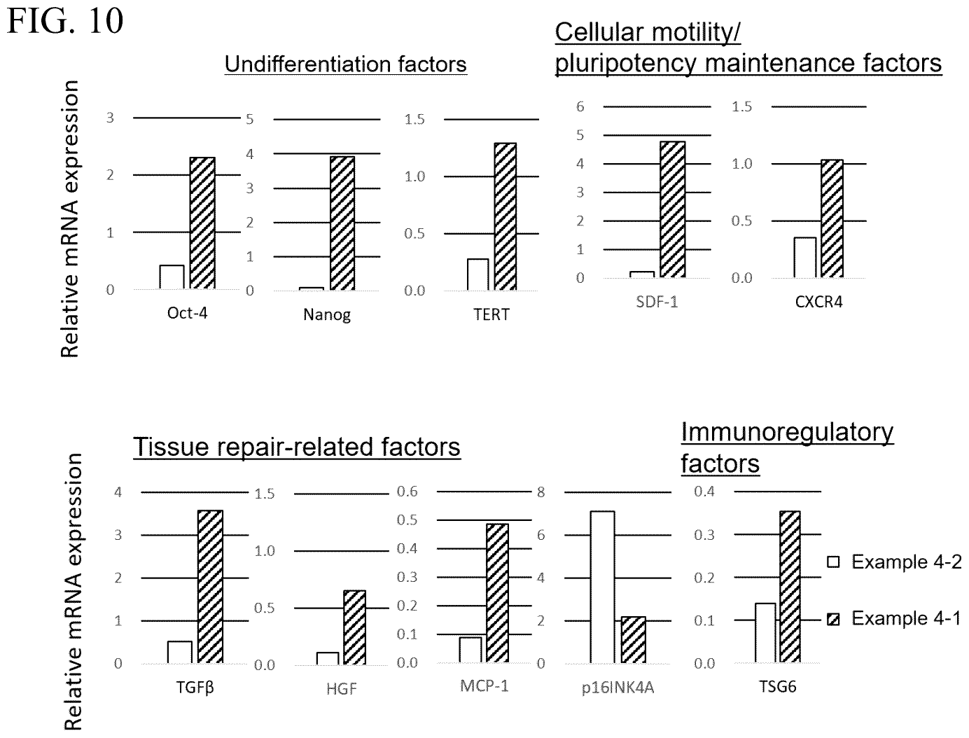

FIG. 10 shows graphs of the results of measuring various expression factors related to the mRNA of the mesenchymal stem cells encapsulated in the hydrogel fibers in Examples 4-1 and 4-2. Specifically, when the day on which culture was started was set to Day 0, the various expression factors related to the mRNA were measured on Day 20.

FIG. 10 shows undifferentiation factors (Oct-4, Nanog, and TERT), cellular motility/pluripotency maintenance factors (SDF-1 and CXCR4), tissue repair-related factors (TGFβ, HGF, and MCP-1), a cell senescence-related factor and tumor suppressor gene (p16INK4A), and an immunoregulatory factor (TSG6). TGFβ, HGF, and MCP-1 are factors that contribute to the repair and the regeneration of a tissue that has been damaged by inflammation or the like.

FIG. 10 shows the expression level of each factor in each Example obtained when an appropriate reference value was normalized as “1”.

Except for p16INK4A, the expression levels of almost all of the above expression factors were higher in Example 4-1 than in Example 4-2. It is thus understood that contribution to the expression levels of the expression factors is greater when the microfibers contain the extracellular matrix (scaffold), which is collagen in the present Example 4-1.

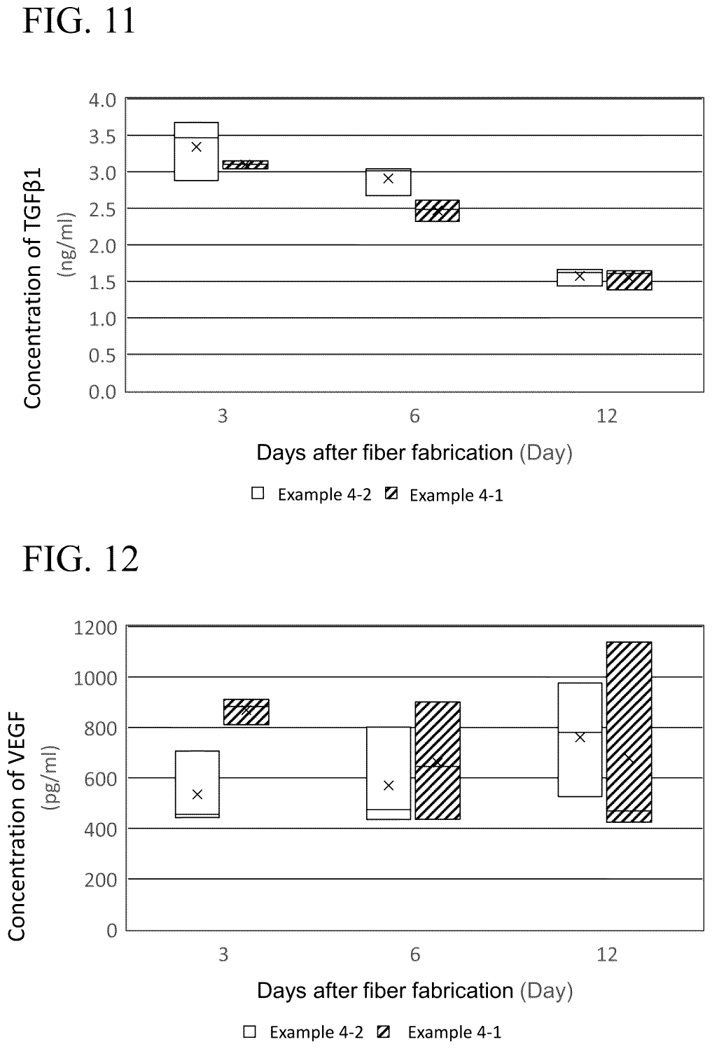

FIG. 11 shows a graph of the results of measuring a tissue-repair factor (TGF-β1) derived from the mesenchymal stem cells encapsulated in the hydrogel fibers in Examples 4-1 and 4-2. The vertical axis in FIG. 11 represents the concentration of TGF-β1 in the medium. The horizontal axis in FIG. 11 represents the number of days that have passed since the preparation of the hydrogel fibers described above (culture period). When the day on which the hydrogel fibers were prepared was set to Day 0, TGF-β1 was measured on Day 3, Day 6, and Day 23. In FIG. 11, the rectangles with oblique lines represent the experimental results obtained with the hydrogel fiber in Example 4-1. The blank rectangles represent the experimental results obtained with the hydrogel fiber in Example 4-2. In each of Examples 4-1 and 4-2, the experiment was conducted with 3 hydrogel fibers. The central value in the longitudinal direction in each rectangle is the mean value of the results of the experiment conducted with the 3 hydrogel fibers. The length of each rectangle in the longitudinal direction represents the standard deviation (dispersion) of the results of the experiment conducted with the 3 hydrogel fibers.

The amounts of TGF-β1 secreted were nearly equal in both Examples 4-1 and 4-2.

In both Examples 4-1 and 4-2, the amounts of TGF-β1 secreted decreased as the number of days (culture period) increased since the preparation of the hydrogel fibers.

FIG. 12 shows a graph of the results of measuring a vascular endothelial growth factor (VEGF) secreted from the mesenchymal stem cells encapsulated in the hydrogel fibers in Examples 4-1 and 4-2. The vertical axis in FIG. 12 represents the concentration of VEGF in the medium. The horizontal axis in FIG. 12 represents the number of days that have passed since the preparation of the hydrogel fibers described above (culture period). In FIG. 12, the rectangles with oblique lines represent the experimental results obtained with the hydrogel fiber in Example 4-1. The blank rectangles represent the experimental results obtained with the hydrogel fiber in Example 4-2. In each of Examples 4-1 and 4-2, the experiment was conducted with 3 hydrogel fibers. The central value in the longitudinal direction in each rectangle is the mean value of the results of the experiment conducted with the 3 hydrogel fibers. The length of each rectangle in the longitudinal direction represents the standard deviation (dispersion) of the results of the experiment conducted with the 3 hydrogel fibers.

When the culture period was relatively short, the amount of VEGF secreted from the hydrogel fibers of Example 4-1 was greater than the amount of VEGF secreted from the hydrogel fibers of Example 4-2. When the culture period was relatively long, the amount of VEGF secreted from the hydrogel fibers of Example 4-1 was nearly equal to the amount of VEGF secreted from the hydrogel fibers of Example 4-2.

The amounts of VEGF secreted remained almost unchanged over a long period of time in both Examples 4-1 and 4-2. Therefore, the amount of VEGF secreted can be maintained over a relatively long period of time by encapsulating the mesenchymal stem cells in the hydrogel fiber. Therefore, the hydrogel fibers in the present Examples can be preferably used for maintaining a vascular endothelial growth factor (VEGF).

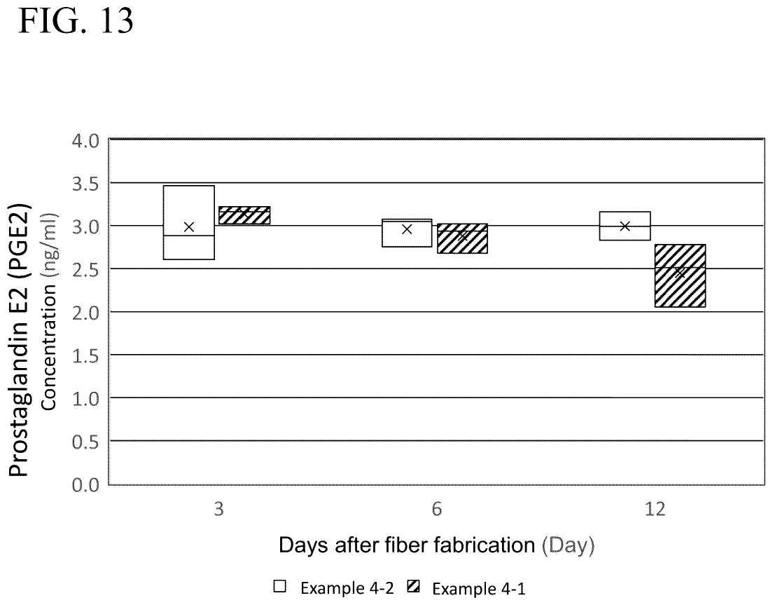

FIG. 13 shows a graph of the results of measuring a factor (PGE2) secreted from the mesenchymal stem cells encapsulated in the hydrogel fibers in Examples 4-1 and 4-2. The vertical axis in FIG. 13 represents the concentration of PGE2 in the medium. The vertical axis in FIG. 13 represents the concentration of PGE2 in the medium. The horizontal axis in FIG. 13 represents the number of days that have passed since the preparation of the hydrogel fibers described above (culture period). In FIG. 13, the rectangles with oblique lines represent the experimental results obtained with the hydrogel fiber in Example 4-1. The blank rectangles represent the experimental results obtained with the hydrogel fiber in Example 4-2. In each of Examples 4-1 and 4-2, the experiment was conducted with 3 hydrogel fibers. The central value in the longitudinal direction in each rectangle is the mean value of the results of the experiment conducted with the 3 hydrogel fibers. The length of each rectangle in the longitudinal direction represents the standard deviation (dispersion) of the results of the experiment conducted with the 3 hydrogel fibers.

The amounts of PGE2 secreted remained almost unchanged over a long period of time in both Examples 4-1 and 4-2. Therefore, the amount of PGE2 secreted can be maintained over a relatively long period of time by encapsulating the mesenchymal stem cells in the hydrogel fiber. Here, PGE2 is known as a factor that strongly suppresses inflammation by acting on immune cells such as a macrophage. It is thus considered that the hydrogel fiber encapsulating the mesenchymal stem cells can be preferably used for suppressing inflammation during transplantation.

The following transplantation into mice was performed as an example of the use of the hydrogel fibers in various Examples described above. However, it should be noted that the use of the hydrogel fibers should not be limited to the following use.

[TNBS Enteritis Model Mice]

### Example 1-1

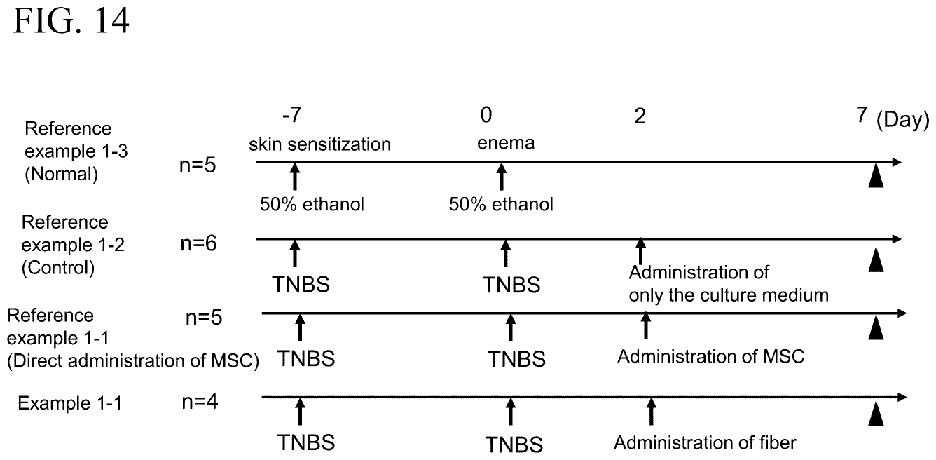

FIG. 14 is a diagram for describing schedules for treatment by the hydrogel fiber in Example 1-1 using TNBS enteritis model mice.

First, TNBS enteritis model mice were prepared by skin-sensitizing Balb/c mice (female, 9 weeks old) with an ethanol solution in which 2,4,6-trinitrobenzenesulfonic acid (TNBS) was dissolved, and performing transanal enema administration of TNBS one week later.

When the day on which the transanal enema administration of TNBS was performed was set to “Day 0”, the hydrogel fibers of Example 1-1 were transplanted into the peritoneal cavities of the model mice on Day 2.

For reference, human umbilical cord-derived mesenchymal stem cells that were not encapsulated in the hydrogel fibers were transplanted into the peritoneal cavities of the model mice as they were (group directly administered with MSCs: Reference Example 1-1).

Furthermore, for reference, when the day on which the transanal enema administration of TNBS was performed was set to “Day 0”, only a serum-free GlutaMAX medium not containing FBS and an antibiotic (containing neither the hydrogel fiber nor the human umbilical cord-derived mesenchymal stem cells) was administered into the peritoneal cavities of the model mice on Day 2 (control group: Reference Example 1-2).

In addition, for reference, Balb/c mice (female, 9 weeks old) were skin-sensitized with only ethanol which was the solvent for TNBS, and transanal enema administration of only ethanol was performed one week later, and then the model mice were observed without transplanting the human umbilical cord-derived mesenchymal stem cells (normal group: Reference Example 1-3).

The value of “n” in FIG. 14 indicates the number of model mouse samples used in each Example and Reference Example.

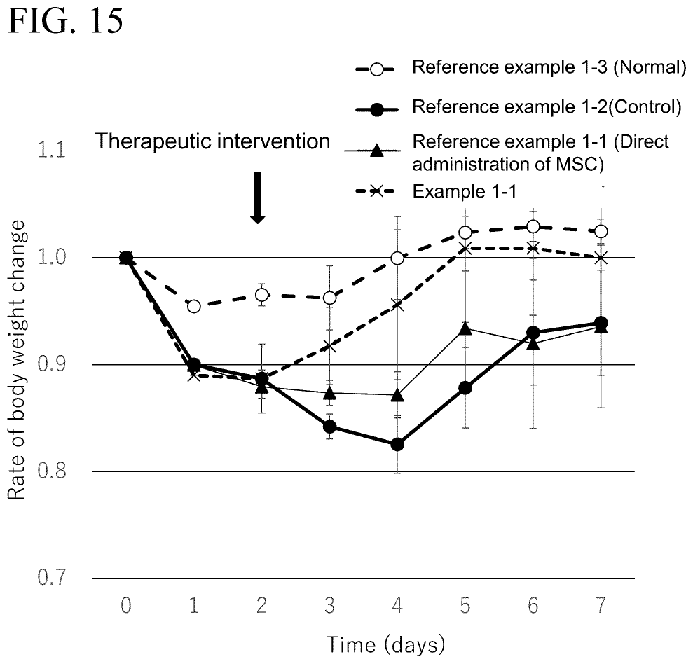

FIG. 15 shows a graph of body weight changes in the TNBS enteritis model mice that have received the various treatments. The vertical axis in FIG. 15 represents numerical values obtained by correcting the body weight of each individual during the observation period by the body weight on Day 0, thus setting the body weight of the model mouse on Day 0 to “1”, whereby normalization was performed so that the rate of body weight change was the same in each Example and Reference Example.

Since exacerbation of enteritis is accompanied by symptoms of diarrhea and hematochezia in the model mice, the body weights of the model mice decrease. Therefore, the body weights of the TNBS enteritis model mice in Example 1-1 and Reference Examples 1-1 and 1-2 become lower than the body weights of the model mice in the normal group in Reference Example 1-3 with the passage of days.

The body weights of the TNBS enteritis model mice in Reference Example 1-2 was significantly lower than the body weights of the model mice in the normal group in Reference Example 1-3.

Meanwhile, the rate of body weight change of the model mice in Example 1-1 was maintained at a higher value than the rates of body weight change of the TNBS enteritis model mice in Reference Examples 1-1 and 1-2. In other words, it is considered that the symptoms of enteritis were alleviated in the model mice into which the hydrogel fibers encapsulating the mesenchymal stem cells were transplanted, compared to the model mice to which the mesenchymal stem cells were directly administered (Reference Example 1-1).

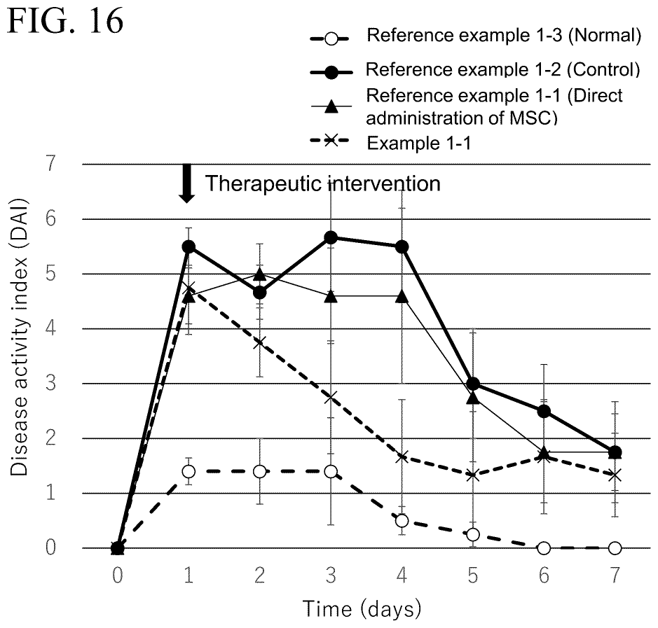

FIG. 16 shows a graph of disease activity indices (DAIs) for the TNBS enteritis model mice that have received the various treatments. DAI is an index of enteritis activity obtained by scoring the rate of body weight loss, diarrhea, and the state of hematochezia in the model mice. In the present specification, the DAI is calculated as follows.
1) Rate of body weight loss (Lo)
Lo≤1%: 0 points
1%<Lo≤5%: 1 point
5%<Lo≤10%: 2 points
10%<Lo≤15%: 3 points
15%<Lo: 4 points
2) Stool consistency
Normal: 0 points
Loose stool: 1 point
Diarrhea: 3 points
3) Hematochezia
None (Negative): 0 points
Hemoccult positive: 2 points
Marked gross bleeding: 4 points

The DAI is calculated by summing up the 3 types of scores, the rate of body weight loss, the stool consistency, and the degree of hematochezia in the model mice. A higher DAI value means that the activity of enteritis is high, that is, enteritis has been exacerbated.

With the exacerbation of enteritis, the DAI for the TNBS enteritis model mice in Reference Example 1-2 becomes higher than the DAI for the model mice in Reference Example 1-3, which is the normal group.

Meanwhile, the DAI for the model mice in Example 1-1 is found to be decreased in an early stage by the transplantation of the hydrogel fibers. It was thus found that the hydrogel fiber containing collagen as the extracellular matrix and the mesenchymal stem cells was effective as a graft.

On the other hand, in the model mice in Reference Example 1-1 to which the mesenchymal stem cells were directly administered, DAI was not decreased as in Reference Example 1-2. It was thus found that the administration of the mesenchymal stem cells encapsulated in the hydrogel fiber was more effective.

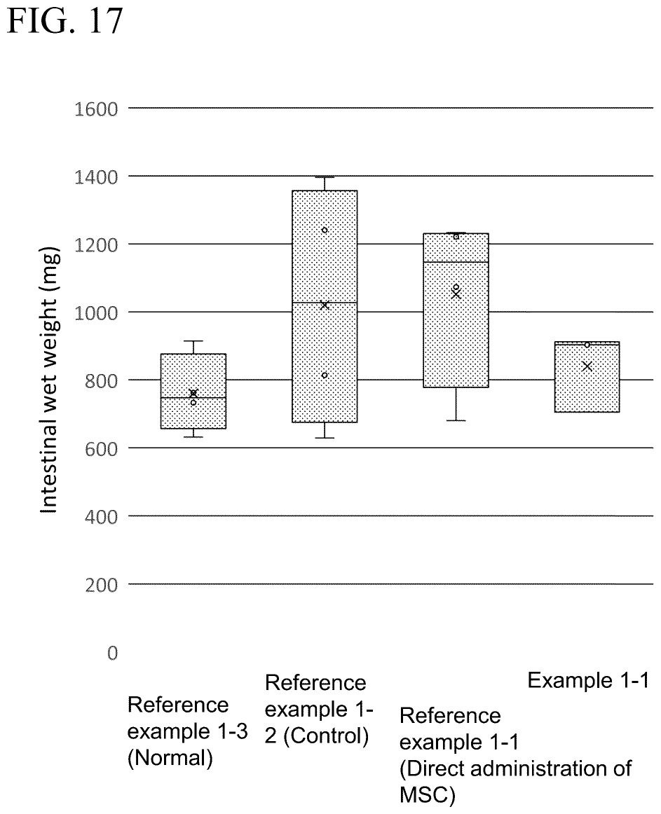

FIG. 17 shows a graph of changes in intestinal wet weights in the TNBS enteritis model mice that have received the various treatments. Specifically, the model mice were dissected on Day 7, and the intestinal wet weight of each model mouse was measured.

When Reference Example 1-3 (normal group) was compared with Reference Example 1-2 (control group), the intestinal wet weight of the model mice in Reference Example 1-2 was greater than the intestinal wet weight of the model mice in Reference Example 1-3. This is considered to be the increase in weight due to inflammatory cell infiltration accompanying the onset of TNBS enteritis.

The intestinal wet weight of the model mice in Example 1-1 into which the hydrogel fibers were transplanted was smaller than the intestinal wet weight of the model mice in Reference Example 1-2. This means that the inflammatory cell infiltration is suppressed in a case where the hydrogel fiber containing collagen (Example 1-1) is transplanted. That is, it is found that the inflammatory cell infiltration is suppressed by a synergistic effect between the hydrogel fiber and the mesenchymal stem cells.

Furthermore, when Example 1-1 is compared with Reference Example 1-1, it is found that the inflammatory cell infiltration is suppressed more in a case where the hydrogel fiber containing collagen (Example 1-1) is transplanted than in a case where the mesenchymal stem cells are directly administered.

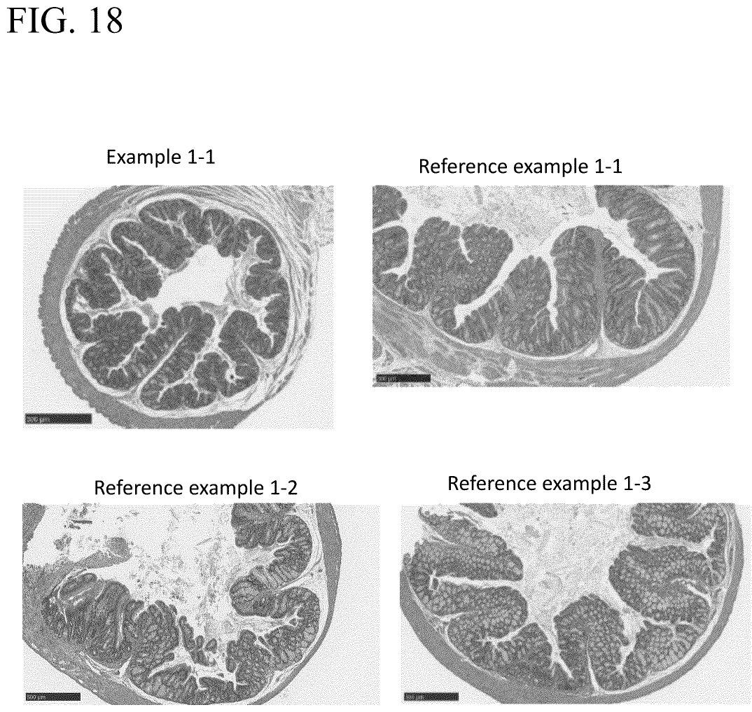

FIG. 18 shows histopathological images (hematoxylin-eosin staining) of proximal colons of the TNBS enteritis model mice that have received the various treatments. Specifically, FIG. 18 shows photographs of distal colons of the model mice dissected on Day 7.

In FIG. 18, the arrangement and heights of the ducts constituting the crypts in Reference Example 1-2 were irregular compared to those in Reference Example 1-3 (normal group), and reduction and irregularity in the goblet cells constituting the ducts were also observed in Reference Example 1-2. In addition, marked inflammatory cell infiltration in the stroma was observed in Reference Example 1-2. On the other hand, a tendency to suppress the degeneration of crypts and the inflammatory cell infiltration was found in Example 1-1. The inflammatory cell infiltration and the degeneration of crypts remain in Reference Example 1-1 (direct administration of mesenchymal stem cells).

There was no significant difference in survival rates among the model mice in Example 1-1 and Reference Examples 1-1 and 1-2 on Day 7.

It is considered that, from the cytokine profile, a characteristic similar to the pathology of Crohn's disease is exhibited by the TNBS enteritis model. TNBS is a hapten that non-specifically binds to various proteins, and it is thus considered that enteritis is caused in TNBS colitis based on a plurality of immune responses. Therefore, it is conceivable that the above Example is effective against, for example, Crohn's disease.

[Chronic Enteritis Model Mice (1)]

### Examples 2-1 to 2-4

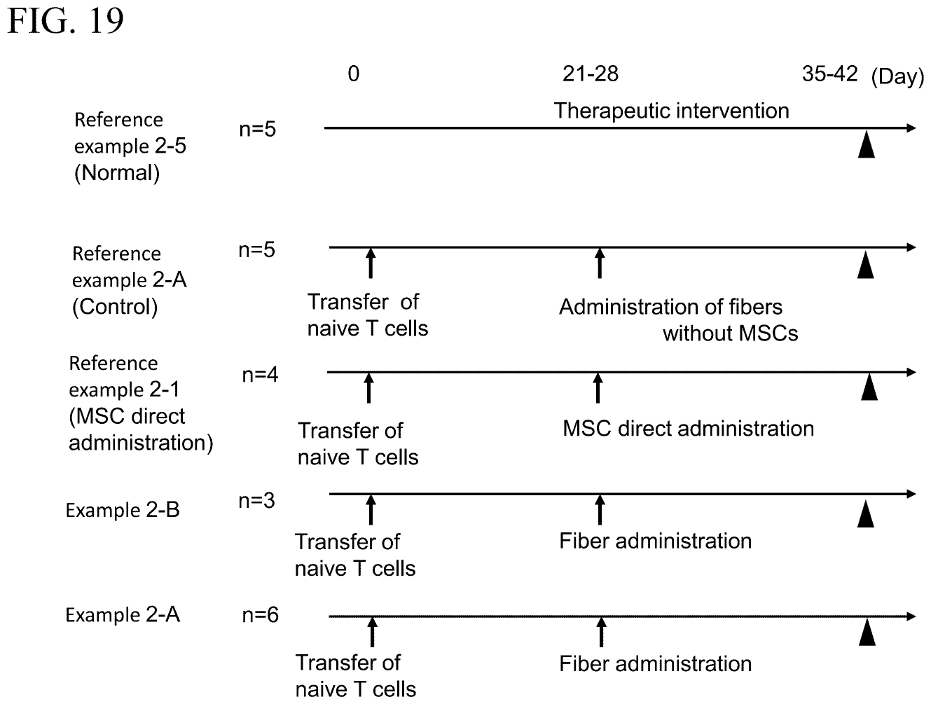

FIG. 19 is a diagram for describing schedules for treatments by the hydrogel fibers using naive T cell transfer enteritis model mice.

First, naive T cells (CD4+CD62L+naive T cells) are isolated from the spleens of Balb/c mice, and the naive T cells are transferred into immunodeficient mice (SCID Mice). As a result, model mice affected with chronic enteritis are obtained.

When the day on which the naive T cells were transferred into the model mice was set to “Day 0”, the hydrogel fibers encapsulating the mesenchymal stem cells (Examples 2-1, 2-2, and 2-4) were transplanted into the peritoneal cavities of the model mice on Day 26. Furthermore, the hydrogel fibers not containing the mesenchymal stem cells (Reference Examples 2-2 and 2-3) were transplanted into the peritoneal cavities of the model mice for reference.

Moreover, model mice were also prepared by directly transplanting the human umbilical cord-derived mesenchymal stem cells that were not encapsulated in the hydrogel fibers into the peritoneal cavities thereof for reference (Reference Example 2-1).

In addition, model mice were also prepared by administering only a cell-free GlutaMAX medium for reference (Reference Example 2-4).

In addition, for reference, model mice not subjected to any treatment (SCID Mice) were observed as well without performing the naive T cell transfer (normal group: Reference Example 2-5).

Next, the results of observing the naive T cell transfer enteritis model mice that have received the various treatments are described. Here, in statistically processing the observation results, the model mice into which the hydrogel fibers in Examples 2-1 and 2-2 were transplanted were treated as the same group. Hereinafter, the group including Examples 2-1 and 2-2 may be referred to as Example 2-A (also refer to Table 2 below).

The hydrogel fibers Example 2-3 were not used for the transplantation. Example 2-4 may be referred to as Example 2-B in Table 2 (also refer to Table 2 below).

Similarly, the model mice in Reference Examples 2-2 and 2-3 into which the hydrogel fibers not containing the cells were transplanted and the model mice in Reference Example 2-4 to which only the cell-free GlutaMAX medium was administered were treated as the same group. Hereinafter, the group including Reference Examples 2-2, 2-3, and 2-4 may be referred to as Reference Example 2-A (also refer to Table 2 below).

TABLE 2

| | | Number | | | | |
|---|---|---|---|---|---|---|
| | | of model | | | | |
| | | mice | Cells | Base material | Hydrogel | Condition |
| Example 2-1 | Example 2-A | 3 | MSC | with native collagen | Calcium alginate | Enteritis transfected |
| Example 2-2 | | 3 | MSC | with native collagen | Barium alginate | with naive T cells |
| Example 2-3 | | 0 | MSC | Medium | Calcium alginate | — |
| Example 2-4 | Example 2-B | 3 | MSC | Medium | Barium alginate | Enteritis transfected |
| | | | | | | with naive T cells |
| Reference | | 4 | MSC | *Direct transplantation | — | Enteritis transfected |
| example 2-1 | | | | | | with naive T cells |
| Reference | Reference | 2 | — | with native collagen | Calcium alginate | Enteritis transfected |
| example 2-2 | example 2-A | | | | | with naive T cells |
| Reference | | 2 | — | with native collagen | Barium alginate | Enteritis transfected |
| example 2-3 | | | | | | with naive T cells |
| Reference | | 1 | *Administration of medium only | | | Enteritis transfected |
| example 2-4 | | | | | | with naive T cells |
| Reference | | 5 | — | — | — | Normal control |
| example 2-5 | | | | | | |

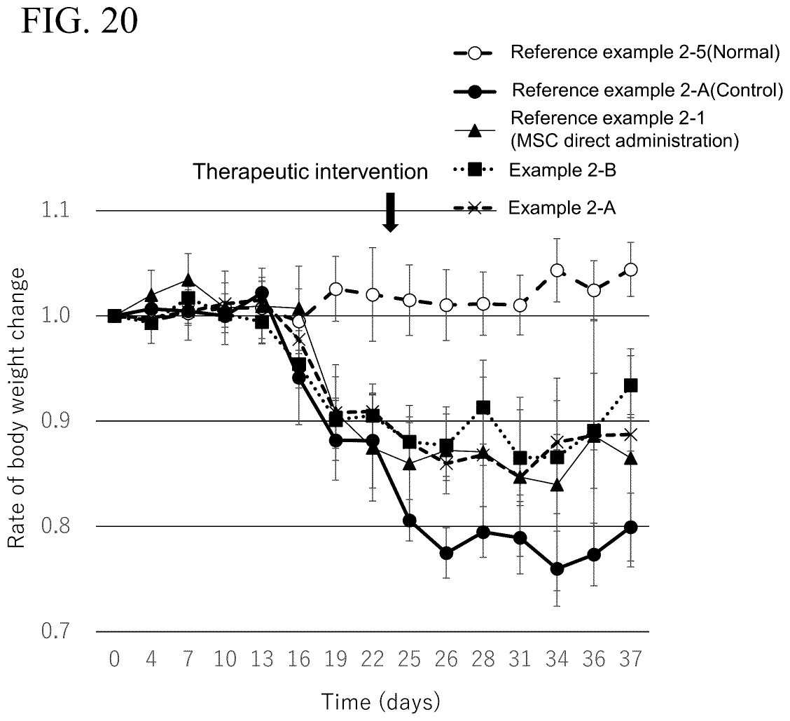

FIG. 20 shows a graph of the rate of body weight change of the naive T cell transfer enteritis model mice that have received the various treatments. The vertical axis in FIG. 20 represents numerical values obtained by correcting the body weight of each individual during the observation period by the body weight on Day 0, thus setting the body weight of the model mouse on Day 0 to “1”, whereby normalization was performed so that the rate of body weight change was the same in each Example and Reference Example.

Since exacerbation of enteritis is accompanied by the symptom of diarrhea in the model mice, the body weights of the model mice decrease. Therefore, the rates of body weight change of the naive T cell transfer enteritis model mice in Examples 2-A and 2-B and Reference Examples 2-A and 2-1 become lower than the body weights of the model mice in Reference Example 2-5 (normal group) with the passage of days.

The rate of body weight change of the model mice in Reference Example 2-A was significantly lower than the rate of body weight change of the model mice in Reference Example 2-5, that is, the model mice not affected with enteritis.

Meanwhile, the rates of body weight change of the model mice in Examples 2-A and 2-B were maintained at higher values than the rates of body weight change of the model mice in Reference Example 2-A. In other words, it is considered that the symptom of enteritis was alleviated in the model mice into which the hydrogel fibers encapsulating the mesenchymal stem cells were transplanted.

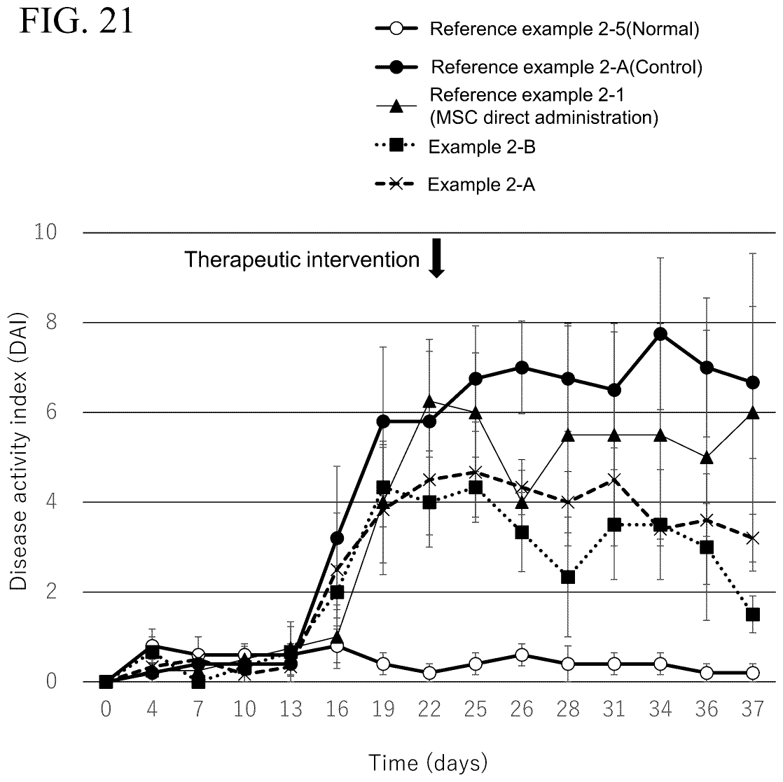

FIG. 21 shows a graph of disease activity indices (DAIs) for the naive T cell transfer enteritis model mice that have received the various treatments. The method for calculating the DAI is as described above.

With the exacerbation of enteritis, the DAI for the naive T cell transfer enteritis model mice in Reference Example 2-A becomes higher than the DAI for the model mice in Reference Example 2-5 (normal group) that are not affected with enteritis.

Meanwhile, the increase in the DAI is found to be suppressed in the model mice in Examples 2-A and 2-B due to the transplantation of the hydrogel fibers. It was thus found that the hydrogel fiber encapsulating the mesenchymal stem cells was effective as a graft.

The survival rate of the model mice in Reference Example 2-1 on Day 47 was 25%. Meanwhile, the survival rates of the model mice in Examples 2-A and 2-B and Reference Example 2-A on Day 47 were 60 to 67%. It was thus found that the survival rate increased due to the administration of the hydrogel fibers encapsulating the mesenchymal stem cells, rather than the direct administration of the mesenchymal stem cells into the model mice.

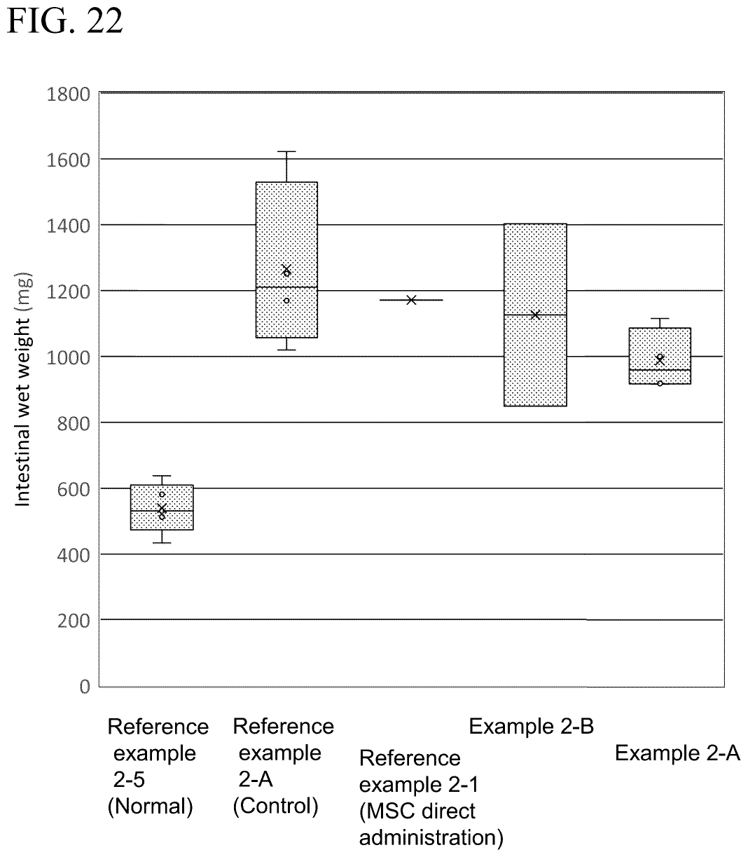

FIG. 22 shows a graph of changes in intestinal wet weights in the naive T cell transfer enteritis model mice that have received the various treatments. Specifically, the model mice were dissected on Day 47, and the intestinal wet weight of each model mouse was measured.

When Reference Example 2-5 (normal group) was compared with Reference Example 2-A, the intestinal wet weight of the model mice in Reference Example 2-A was greater than the intestinal wet weight of the model mice in Reference Example 2-5. This is considered to be the increase in weight due to inflammatory cell infiltration accompanying the onset of enteritis.

The intestinal wet weight of the model mice in Reference Example 2-1 to which the mesenchymal stem cells were directly administered was slightly smaller than the intestinal wet weight of the model mice in Reference Example 2-A. This means that the inflammatory cell infiltration tends to be suppressed by the administration of the mesenchymal stem cells.

The intestinal wet weight of the model mice in Example 2-B into which the hydrogel fibers were transplanted was smaller than the intestinal wet weights of the model mice in Reference Examples 2-A and 2-1. This means that the inflammatory cell infiltration is suppressed by the transplantation of the hydrogel fibers encapsulating the mesenchymal stem cells.

The intestinal wet weight of the model mice in Example 2-A into which the hydrogel fibers were transplanted was smaller than the intestinal wet weight of the model mice in Example 2-B. This means that the hydrogel fiber containing the extracellular matrix, particularly, collagen, is more preferred.

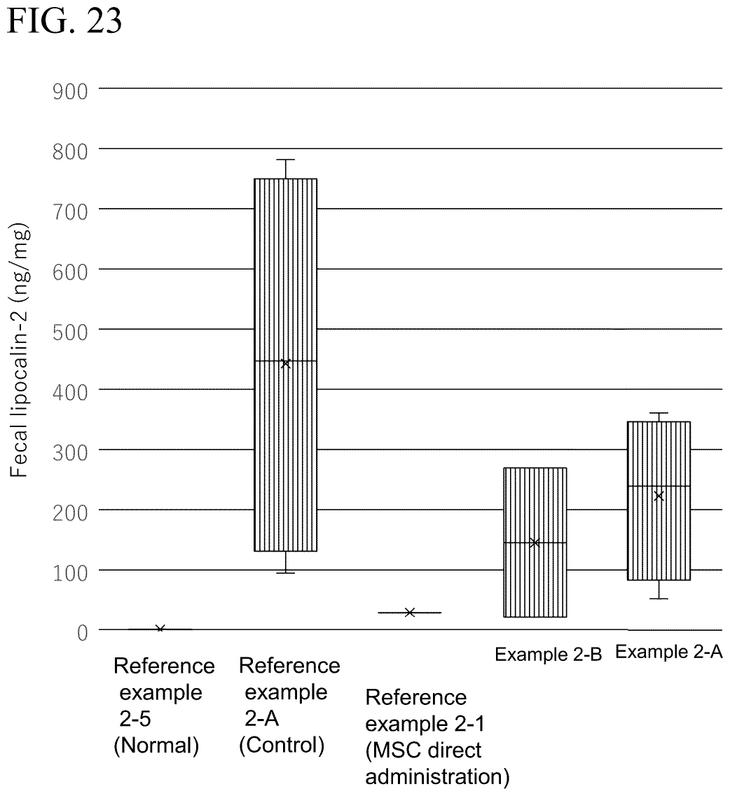

FIG. 23 shows a graph of the results of measuring neutrophil gelatinase-associated lipocalin (LPN-2) in stools of the naive T cell transfer enteritis model mice that have received the various treatments. Specifically, the model mice were dissected on Day 47, and the amount of the neutrophil gelatinase-associated lipocalin in the stool collected from each model mouse was measured. The neutrophil gelatinase-associated lipocalin is involved in an innate immune response in a bacterial infection. Specifically, the concentration of LPN-2 increases due to induction of intestinal inflammation. Therefore, a lower concentration of the neutrophil gelatinase-associated lipocalin is preferred.

When Reference Example 2-5 (normal group) was compared with Reference Example 2-A, the LPN-2 concentration in the model mice in Reference Example 2-A was higher than the LPN-2 concentration in the model mice in Reference Example 2-5. This is considered to be due to the influence of the onset of chronic enteritis in the model mice in Reference Example 2-A.

The LPN-2 concentrations in the model mice in Examples 2-A and 2-B were lower than the LPN-2 concentration in the model mice in Reference Example 2-A. This is considered to be due to the suppression of intestinal inflammation by the transplantation of the hydrogel fibers encapsulating the mesenchymal stem cells.

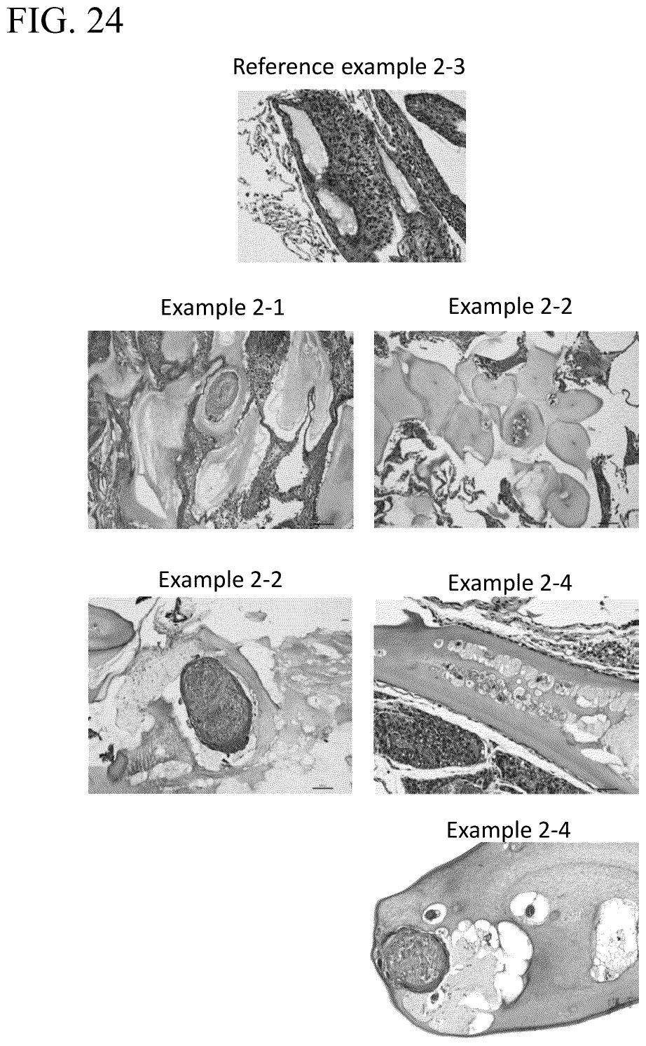

FIG. 24 shows photographs of the states in which the hydrogel fibers transplanted into the naive T cell transfer enteritis model mice were extracted on Day 47 after the onset of enteritis.

When the hydrogel fibers in Reference Example 2-4 were extracted after being transplanted, no cells were found inside the hydrogel. Furthermore, thick inflammatory cell infiltration and fibrogenesis were observed around the hydrogel.

When the hydrogel fibers in Examples 2-1, 2-2, and 2-4 were extracted after being transplanted, the mesenchymal stem cells were found inside the hydrogel. In addition, the inflammatory cell infiltration and the fibrogenesis that occurred around the hydrogel were lessened compared to the case of Reference Example 2-4.

When the hydrogel fibers in Examples 2-1 and 2-2 (Example 2-A) were extracted after being transplanted, the mesenchymal stem cells were found inside the hydrogel. In addition, the inflammatory cell infiltration and the fibrogenesis that occurred around the hydrogel were lessened compared to the case of Example 2-4 (Example 2-B).

In the naive T cell transfer enteritis model, when naive T cells (CD4+CD62L+naive T cells) are transferred into an immunodeficient mouse, the T cells are stimulated and activated by enteric bacteria, thus causing the onset of enteritis. The naive T cell transfer enteritis model is known as a model associated with the regulation of immune cells. In addition, the naive T cell transfer enteritis model is also being studied as a model for ulcerative colitis and Crohn's disease. Therefore, it is conceivable that Examples described above can be suitably used for regulating immune cells or against ulcerative colitis and Crohn's disease.

[Chronic Enteritis Model Mice (2)]

### Examples 3-1 to 3-3

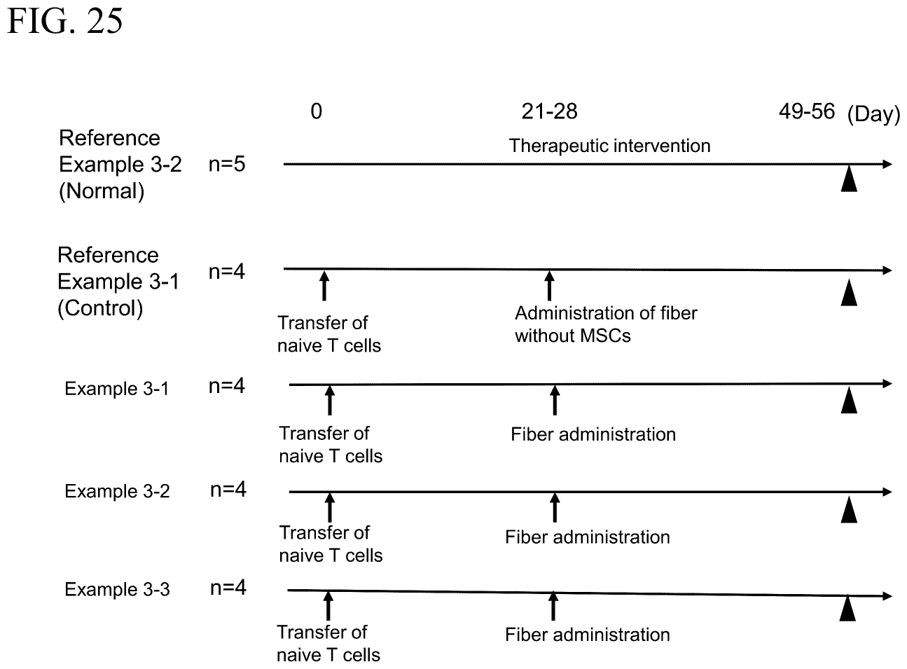

FIG. 25 is a diagram for describing schedules for treatments by the hydrogel fibers using naive T cell transfer enteritis model mice.

First, naive T cells (CD4+CD62L+naive T cells) are isolated from the spleens of Balb/c mice, and the naive T cells are transferred into model mice (SCID Mice). As a result, chronic enteritis model mice are obtained.

When the day on which the naive T cells were transferred into the model mice was set to “Day 0”, the hydrogel fibers encapsulating the mesenchymal stem cells (Examples 3-1 to 3-3) or the hydrogel fibers not containing the mesenchymal stem cells (Reference Example 3-1) were transplanted into the peritoneal cavities of the model mice on Day 26.

In addition, for reference, model mice not subjected to the naive T cell transfer (SCID Mice) were observed as well (normal group: Reference Example 3-2).

The number of model mouse samples used in each Example and each Reference Example is indicated by the numerical value of “n” in FIG. 25.

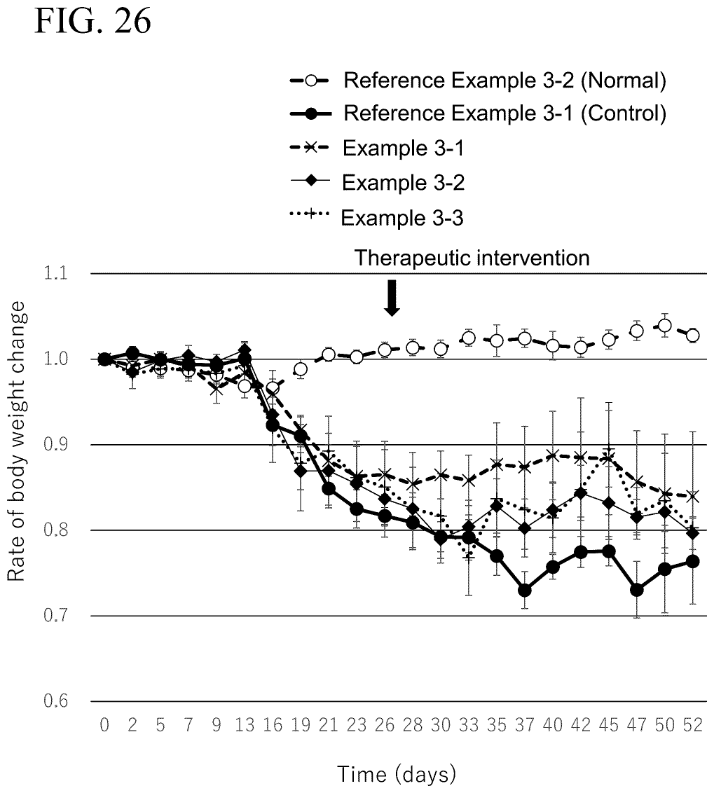

FIG. 26 shows a graph of rates of body weight change of the naive T cell transfer enteritis model mice that have received the various treatments. The vertical axis in FIG. 26 represents numerical values obtained by correcting the body weight of each individual during the observation period by the body weight on Day 0, thus setting the body weight of the model mouse on Day 0 to “1”, whereby normalization was performed so that the rates of body weight change was the same in each Example and Reference Example.

Since exacerbation of enteritis is accompanied by the symptom of diarrhea in the model mice, the body weights of the model mice decrease. Therefore, the rates of body weight change of the model mice in Examples 3-1 to 3-3 and Reference Example 3-1 became lower than the rates of body weight change of the model mice in Reference Example 3-2 with the passage of days.

The rate of body weight change of the model mice in Reference Example 3-1 was significantly lower than the rate of body weight change of the model mice in Reference Example 3-2, that is, the model mice not affected with enteritis.

Meanwhile, the rates of body weight change of the model mice in Examples 3-1 to 3-3 were maintained at higher values than the rates of body weight change of the model mice in Reference Example 3-1. In other words, it is considered that the symptom of enteritis was alleviated in the model mice into which the hydrogel fibers encapsulating the mesenchymal stem cells were transplanted, compared to the model mice into which the hydrogel fibers not containing the mesenchymal stem cells were transplanted (Reference Example 3-1).

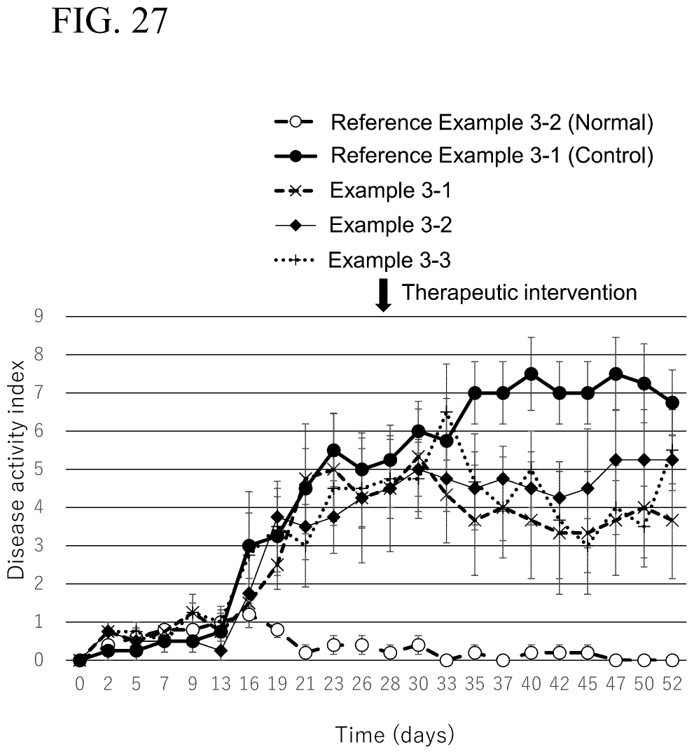

FIG. 27 shows a graph of disease activity indices (DAIs) for the naive T cell transfer enteritis model mice that have received the various treatments. The method for calculating the DAI is as described above.

With the exacerbation of enteritis, the DAIs for the naive T cell transfer enteritis model mice in Examples 3-1 to 3-3 and Reference Example 3-1 become higher than the DAI for the model mice in Reference Example 3-2 (normal group) that are not affected with enteritis.

Meanwhile, the increase in the DAI is found to be suppressed in the model mice in Examples 3-1 to 3-3 compared to the model mice in Reference Example 3-1 by the transplantation of the hydrogel fibers. It was thus found that the hydrogel fiber encapsulating the mesenchymal stem cells was effective as a graft.

The survival rates of the model mice in Examples 3-1 and 3-2 on Day 52 were 75% and 100%, respectively. Meanwhile, the survival rate of the model mice in Example 3-3 on Day 52 was 50%. Therefore, it was found that the administration of the hydrogel fiber containing atelocollagen or fibronectin as the extracellular matrix rises the survival rate more than the administration of the hydrogel fiber containing laminin.

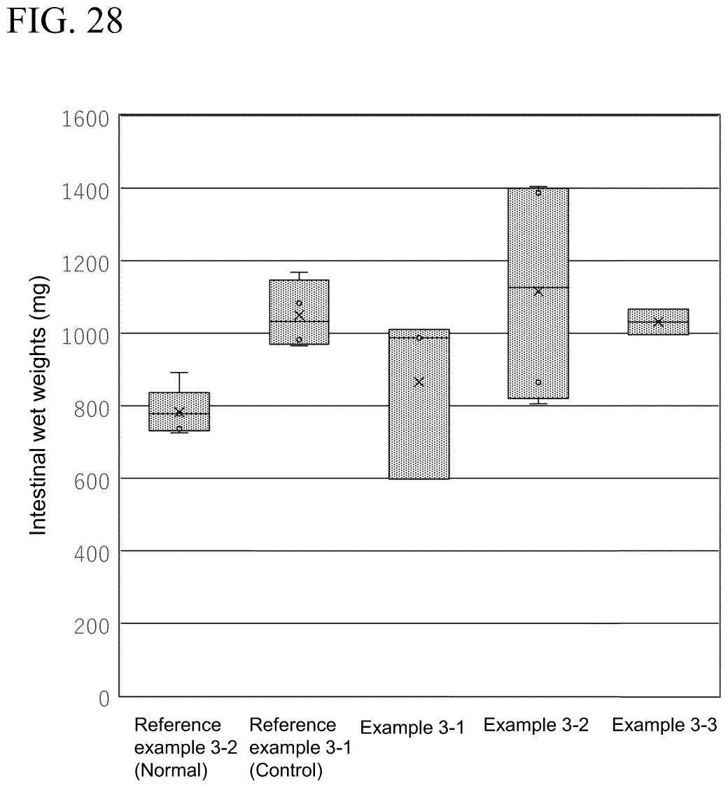

FIG. 28 shows a graph of changes in intestinal wet weights in the naive T cell transfer enteritis model mice that have received the various treatments. Specifically, the model mice were dissected on Day 52, and the intestinal wet weight of each model mouse was measured.

When Reference Example 3-2 (normal group) was compared with Reference Example 3-1, the intestinal wet weight of the model mice in Reference Example 3-1 was greater than the intestinal wet weight of the model mice in Reference Example 3-2. This is considered to be the increase in weight due to inflammatory cell infiltration accompanying the onset of enteritis.

The intestinal wet weight of the model mice in Example 3-1 into which the hydrogel fibers were transplanted was smaller than the intestinal wet weight of the model mice in Reference Example 3-1. This means that the inflammatory cell infiltration is suppressed in a case where the hydrogel fiber containing collagen is transplanted (Example 3-1).

The intestinal wet weights of the model mice in Examples 3-2 and 3-3 into which the hydrogel fibers were transplanted were nearly equal to the intestinal wet weight in Reference Example 3-1.

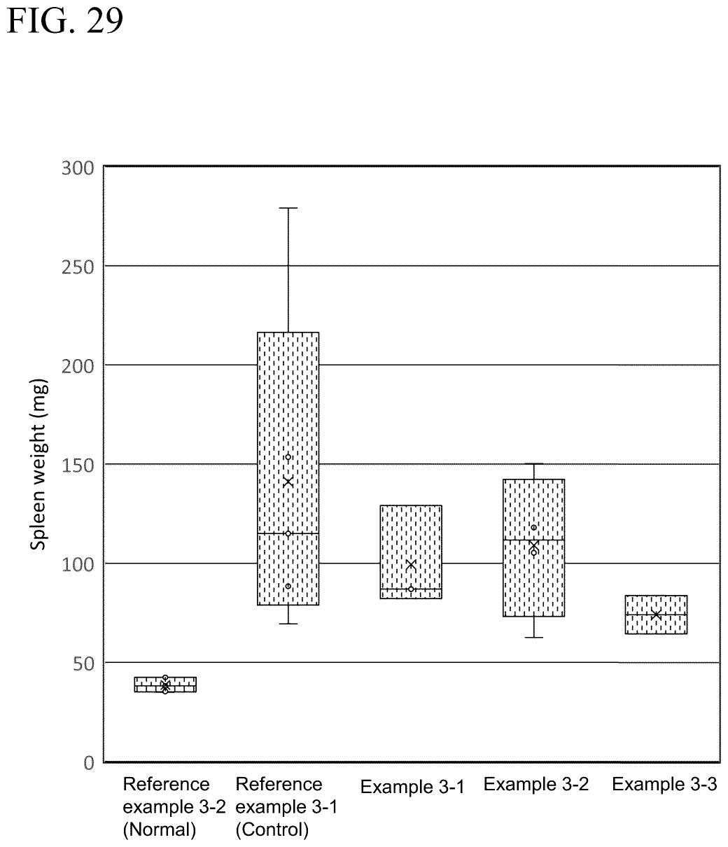

FIG. 29 shows a graph of changes in spleen weights in the naive T cell transfer enteritis model mice that have received the various treatments. Specifically, the model mice were dissected on Day 52, and the spleen weight of each model mouse was measured. Spleen is swollen and increases in weight due to an increased inflammatory reaction that is associated with enteritis.

When Reference Example 3-2 (normal group) was compared with Reference Example 3-1, the spleen weight of the model mice in Reference Example 3-1 was greater than the spleen weight of the model mice in Reference Example 3-2.

The spleen weights of the model mice in Examples 3-1 to 3-3 into which the hydrogel fibers were transplanted were smaller than the spleen weight of the model mice in Reference Example 3-1. This means that the swelling of the spleen associated with the increased inflammatory reaction is suppressed in a case where the hydrogel fiber containing atelocollagen, fibronectin, or laminin (Examples 3-1 to 3-3) is transplanted.

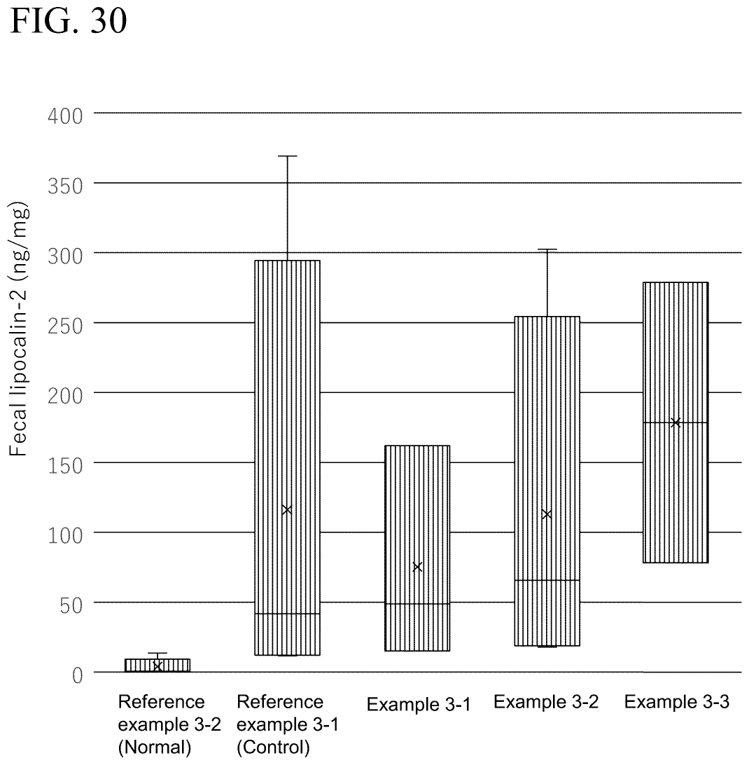

FIG. 30 shows a graph of the results of measuring neutrophil gelatinase-associated lipocalin (LPN-2) in stools of the naive T cell transfer enteritis model mice that have received the various treatments. Specifically, the model mice were dissected on Day 52, and the amount of the neutrophil gelatinase-associated lipocalin in the stool collected from each model mouse was measured.

When Reference Example 3-2 (normal group) was compared with Reference Example 3-1, the LPN-2 concentration in the model mice in Reference Example 3-1 was higher than the LPN-2 concentration in the model mice in Reference Example 3-2. This is considered to be due to the influence of the onset of chronic enteritis in the model mice in Reference Example 3-1.

The LPN-2 concentration in the model mice in Example 3-1 was lower than the LPN-2 concentration in the model mice in Reference Example 3-1. This is considered to be due to the suppression of intestinal inflammation by the transplantation of the hydrogel fibers encapsulating the mesenchymal stem cells, which contain atelocollagen as the base material.

The LPN-2 concentrations in Examples 3-2 and 3-3 were nearly equal to the LPN-2 concentration in Reference Example 3-1. This means that the secretion of LPN-2 from the inflammatory cells in the intestine is suppressed in a case where the hydrogel fiber containing collagen is transplanted (Example 3-1).

[DSS Enteritis Model Mice]

### Example 4-1

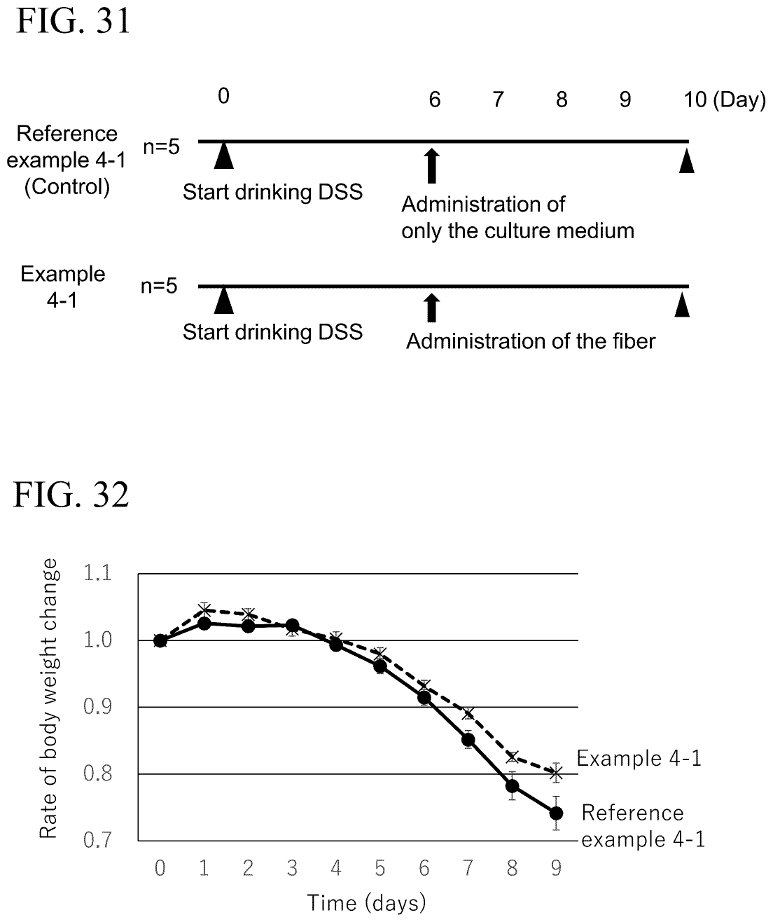

FIG. 31 is a diagram for describing schedules for treatment by the hydrogel fiber using dextran sulfate sodium (DSS)-induced enteritis model mice.

First, C57BL/6 mice (female, 9 weeks old) were allowed to drink dextran sulfate (DSS) ad libitum to prepare DSS enteritis model mice.

During the acute phase of the DSS enteritis, the hydrogel fibers in Example 4-1 that contained collagen were administered into the peritoneal cavities of the prepared model mice. Specifically, when the day on which the model mice were allowed to drink dextran sulfate (DSS) ad libitum was designated as Day 0, the model mice were administered with the hydrogel fibers on Day 6.

Furthermore, only the GlutaMAX medium not containing FBS and the antibiotic (cell-free) was administered as Reference Example 4-1. The time of the administration was Day 6.

“n” in FIG. 31 represents the number of model mouse samples that were prepared.

FIG. 32 shows a graph of rates of body weight change of the DSS enteritis model mice that have received the various treatments. The vertical axis in FIG. 32 represents numerical values obtained by correcting the body weight of each individual during the observation period by the body weight on Day 0, thus setting the body weight of the model mouse on Day 0 to “1”, whereby normalization was performed so that the rates of body weight change was the same in each Example and Reference Example.

The rate of body weight change of the model mice in Example 4-1 was maintained at higher values than the rate of body weight change of the model mice in Reference Example 4-1. In other words, it is considered that the symptom of enteritis was alleviated in the model mice into which the hydrogel fibers encapsulating the mesenchymal stem cells were transplanted, compared to Reference Example 4-1.

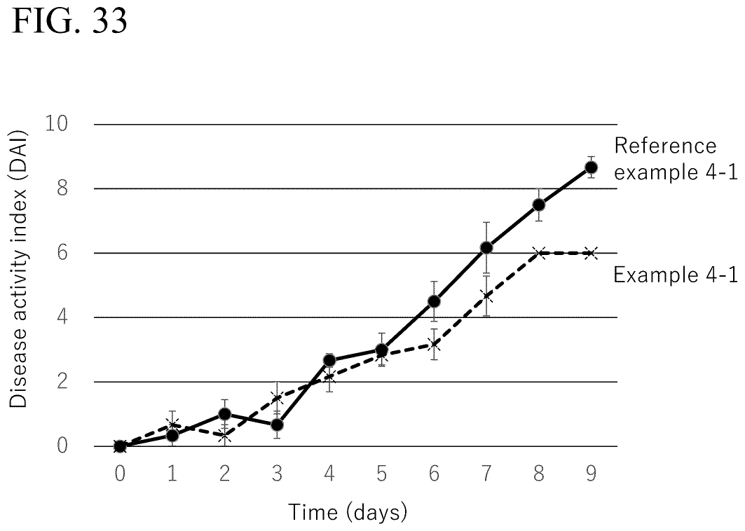

FIG. 33 shows a graph of disease activity indices (DAIS) for the DSS enteritis model mice that have received the various treatments. The DAI was calculated as described above.

The increase in the DAI is found to be suppressed more in the model mice in Example 4-1 than in the model mice in Reference Example 4-1 due to the transplantation of the hydrogel fibers.

From the results shown in FIGS. 32 and 33, it is found that the administration of the hydrogel fiber containing collagen and encapsulating the mesenchymal stem cells is suitable for the treatment or prevention.

The survival rate of the model mice in Example 4-1 on Day 9 was 80%, which was higher than the survival rate of the model mice in Reference Example 4-1 on Day 9, which was 33%.

The DSS enteritis model described above is known as a model produced as a result of an impaired mucosal epithelial function. It is considered that a mucosal barrier function is impaired by the drinking of DSS, and permeability to an antigenic substance derived from bacteria or food is increased, thus causing an abnormality in the mucosal immune system. Therefore, it is implied that the above Example is effective against abnormalities in the mucosal barrier containing epithelial cells and inflammatory cells. In addition, the DSS enteritis model is similar to the pathology of human IBD and has particularly attracted attention as an evaluation model for ulcerative colitis. Therefore, it is conceivable that the above Example is particularly effective against ulcerative colitis.

[Hydrogel Fiber Characteristic Analysis (5)]

### Examples 1-1 and 1-2

The cases of immersing and culturing the mesenchymal stem cells encapsulated in the hydrogel fibers in Examples 1-1 and 1-2 in the GlutaMAX medium containing FBS and the antibiotic together with the fibers were compared with the case of performing 2-dimensional culture of the mesenchymal stem cells without encapsulating the cells in the hydrogel fibers (Reference Example 1-1). Here, Examples 1-1 and 1-2 and Reference Example 1-1 are as described above.

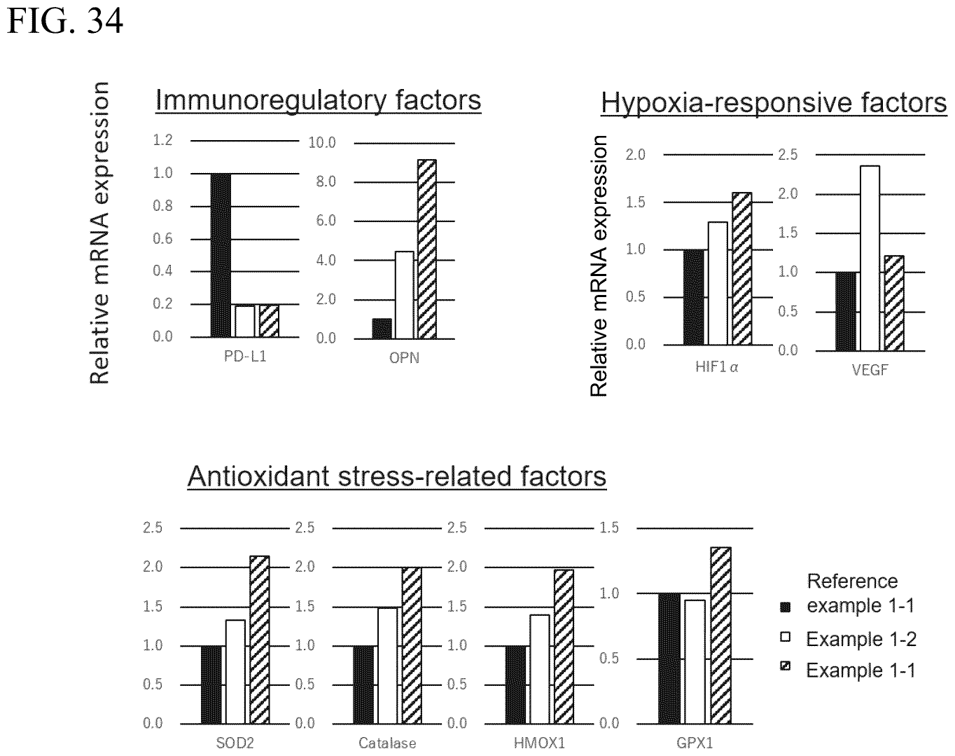

FIG. 34 shows graphs of the results of measuring various expression factors related to the mRNA of the mesenchymal stem cells encapsulated in the hydrogel fibers in Examples 1-1 and 1-2. The vertical axes represent the ratios obtained when the value for the mesenchymal stem cells subjected to the 2-dimensional culture (Reference Example 1-1) was normalized as “1”. Note that, in FIG. 34, Reference Example 1-1 indicates the results obtained by collecting the cells after culturing the cells for 72 hours and performing the measurement, and each Example indicates the results obtained by performing the measurement 18 days after the preparation of the hydrogel fibers.

FIG. 34 shows the results obtained by conducting additional experiments on factors other than the functional factors shown in FIG. 4. FIG. 34 shows immunoregulatory factors (PD-L1 and OPN), hypoxia-responsive factors (HIF1α and VEGF), and antioxidant stress-related factors (SOD2, Catalase, HMOX1, and GPX1).

The expression levels of the factors in the mesenchymal stem cells encapsulated in the hydrogel fibers in Examples 1-1 and 1-2 were equal to or higher than the expression levels of the factors in the 2-dimensional culture (Reference Example 1-1), except for PD-L1. It is thus found that the encapsulation of the mesenchymal stem cells in the hydrogel can contribute to the increase of a number of expression factors related to the mRNA.

For example, the expression levels of the antioxidant stress-related factors in the mesenchymal stem cells encapsulated in the hydrogel fibers in Examples 1-1 and 1-2 are generally higher than the expression levels of the antioxidant stress-related factors in Reference Example 1-1. It is thus considered that the hydrogel fibers in Examples 1-1 and 1-2 can be used as enhancing agents for the expressions of the antioxidant stress-related factors.

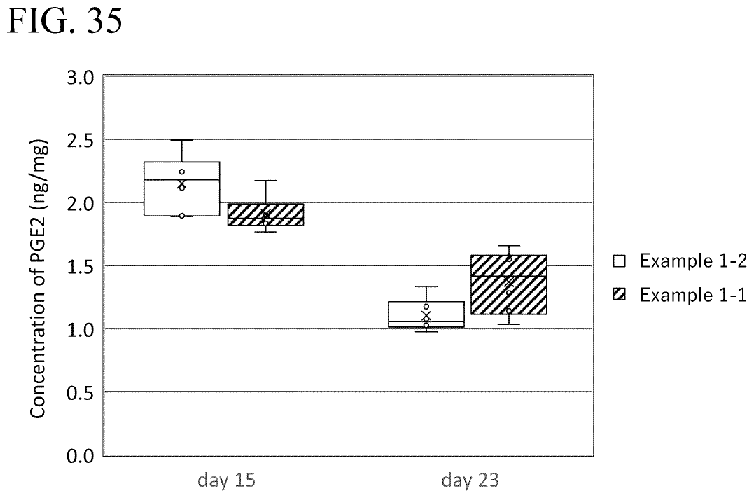

FIG. 35 shows a graph of the concentrations of prostaglandin E2 secreted from the mesenchymal stem cells encapsulated in the hydrogel fibers in Examples 1-1 and 1-2. FIG. 35 shows the prostaglandin E2 concentrations in the culture mediums on Day 15 and Day 23 since the start of the culture of the mesenchymal stem cells encapsulated in the hydrogel fibers.

From FIG. 35, it is found that the prostaglandin E2 concentrations do not decrease significantly on Day 23, and the prostaglandin E2 concentrations are maintained over a long period of time.

Next, the effects on macrophage cell proliferation and activity according to Examples 1-1 and 1-2 are described. First, a macrophage cell line RAW264.7 was induced with lipopolysaccharide (LPS), and 6 hours later, the culture supernatants of the culture medium used in Examples 1-1 and 1-2 were added to RAW264.7. Here, the culture supernatant in each Example is a culture supernatant extracted from the culture medium 24 hours after the start of the culture of the mesenchymal stem cells.

24 hours after the addition of the culture supernatant, the expression levels of M1 macrophage-related factors, M2 macrophage-related factors, and an antioxidant stress-related factor extracted from the macrophage cell line RAW264.7 were measured.

In addition, as Reference Example 1-4, the macrophage cell line RAW264.7 was not induced with lipopolysaccharide (LPS), and the expression levels of the M1 macrophage-related factors, the M2 macrophage-related factors, and the antioxidant stress-related factor extracted from the macrophage cell line RAW264.7 were measured.

Furthermore, as Reference Example 1-5, 6 hours after the induction of the macrophage cell line RAW264.7 with lipopolysaccharide (LPS), only a serum-free GlutaMAX medium that does not contain FBS and an antibiotic was added. 24 hours after the addition of the GlutaMAX medium, the expression levels of the M1 macrophage-related factors, the M2 macrophage-related factors, and the antioxidant stress-related factor extracted from the macrophage cell line RAW264.7 were measured.

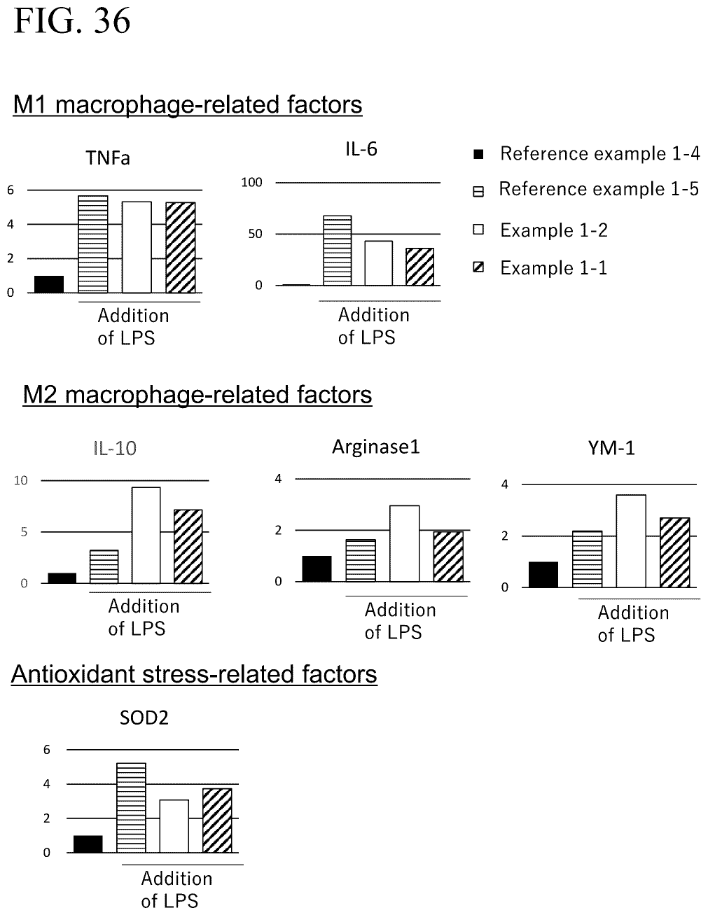

FIG. 36 is a diagram for describing analysis of changes in cell phenotypes in the macrophage cell line RAW264.7 induced with LPS by humoral factors derived from the mesenchymal stem cells in Examples 1-1 and 1-2. The vertical axes represent the expression levels of the various factors in each Example and Reference Example when the expression levels of the various factors in Reference Example 1 were normalized as “1”.

FIG. 36 shows the expression levels of TNFa and IL6 as the M1 macrophage-related factors. FIG. 36 also shows the expression levels of IL-10, Arginase 1, and YM-1 as the M2 macrophage-related factors.

IL-6 showing the M1 phenotype is lower in Examples 1-1 and 1-2 than in Reference Example 1-5 due to the addition of the culture supernatants extracted from the culture medium of the hydrogel fibers. On the other hand, the expression levels of IL-10, Arginase 1, and YM-1 showing the M2 phenotype were higher in Examples 1-1 and 1-2 in Examples 1-1 and 1-2 than in Reference Example 1-5. Therefore, it is conceivable that the hydrogel fibers and the culture supernatants thereof in Examples 1-1 and 1-2 are suitable as enhancing agents for the expressions of the M2 macrophage-related factors.

FIG. 36 also shows the expression level of SOD2 as the antioxidant stress-related factor. The expression levels of SOD2 are lower in Examples 1-1 and 1-2 than in Reference Example 1-5 due to the addition of the culture supernatants extracted from the culture medium of the hydrogel fibers. Therefore, it is conceivable that the hydrogel fibers and the culture supernatants thereof in Examples 1-1 and 1-2 can be used as inhibitors of the expression of the antioxidant stress-related factor.

It is conceivable from such results that the hydrogel fibers and the culture supernatants thereof in Examples 1-1 and 1-2 have the effect of suppressing the cell proliferation or activity of macrophage. Therefore, it is conceivable that the hydrogel fibers and the culture supernatants thereof in Examples 1-1 and 1-2 can be used as inhibitors of the cell proliferation or activity of macrophage.

Next, the results of analyzing a cellular protection effect of humoral factors derived from the mesenchymal stem cells in the hydrogel fibers according to Examples 1-1 and 1-2 on an intestinal epithelial cell line IEC-6 induced with TNFα are described.

6 hours after the induction of the intestinal epithelial cell line IEC-6 with TNFα, the culture supernatants of the culture medium used in Examples 1-1 and 1-2 were added. Here, the culture supernatant in each Example is a culture supernatant extracted from the culture medium 24 hours after the start of the culture of the mesenchymal stem cells. 24 hours after the addition of the culture supernatants, LDH production amount measurement and apoptosis analysis were performed.

In addition, as Reference Example 1-6, the LDH production amount measurement and the apoptosis analysis were performed on the intestinal epithelial cell line IEC-6 that was not induced with TNFα.

Furthermore, as Reference Example 1-7, 6 hours after the induction of the intestinal epithelial cell line IEC-6 with TNFα, only a serum-free GlutaMAX medium that did not contain FBS and the antibiotic was added. 24 hours after the addition of the GlutaMAX medium, the LDH production amount measurement and the apoptosis analysis were performed.

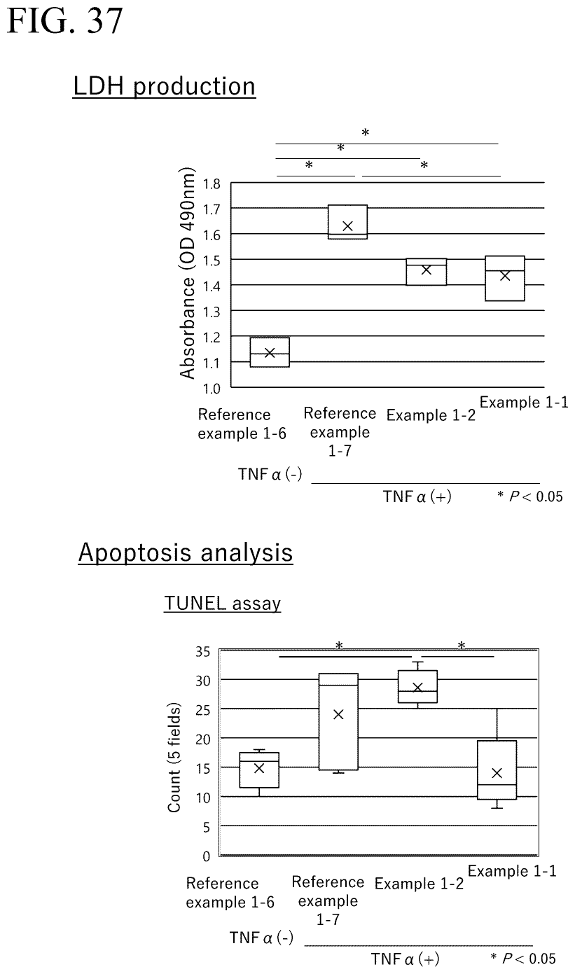

FIG. 37 is a diagram for describing the analysis of the cellular protection effect of the humoral factors derived from the mesenchymal stem cells in Examples 1-1 and 1-2 on the intestinal epithelial cell line IEC-6 induced with TNFα. In the intestinal epithelial cells induced with TNFα, the LDH production and the epithelial cell apoptosis due to cellular damage were suppressed more in Examples 1-1 and 1-2 than in Reference Example 1-7.

[Hydrogel Fiber Characteristic Analysis (6)]

### Examples 2-1, 2-2, 2-3, and 2-4

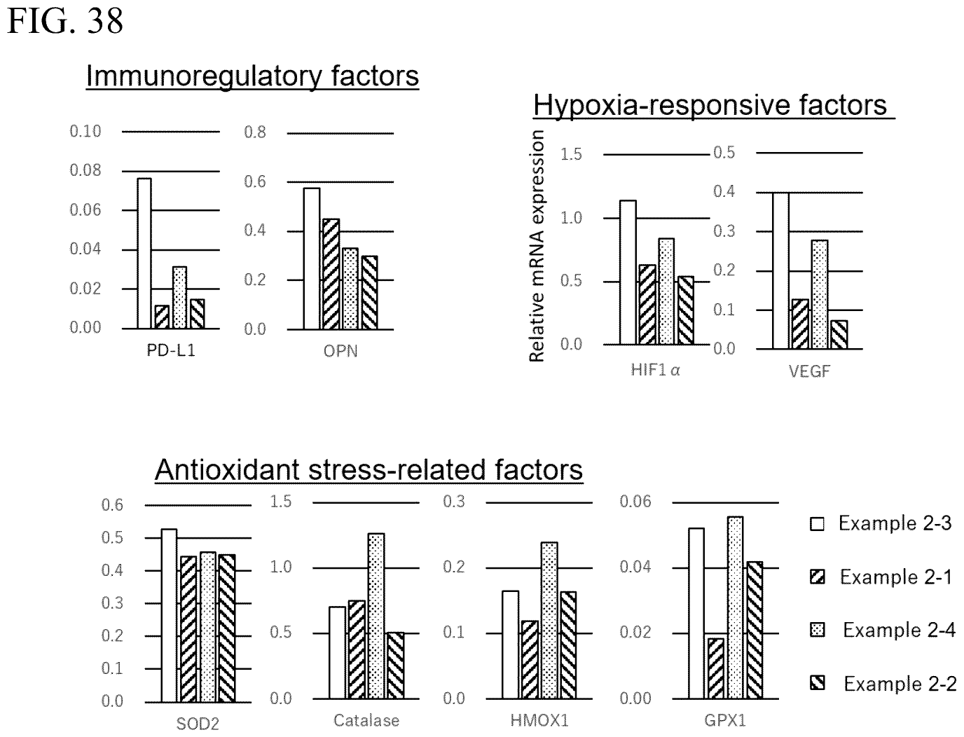

FIG. 38 shows graphs of the results of measuring various expression factors related to the mRNA of the mesenchymal stem cells encapsulated in the hydrogel fibers in Examples 2-1 to 2-4. Here, the descriptions of the hydrogel fibers in Example 2-1 to 2-4 are as described above. In FIG. 38, each Example indicates the results obtained by performing measurement on Day 30 after the preparation of the hydrogel fibers.

FIG. 38 shows the results obtained by conducting additional experiments on factors other than the functional factors shown in FIG. 6. FIG. 38 shows immunoregulatory factors (PD-L1 and OPN), hypoxia-responsive factors (HIF1α and VEGF), and antioxidant stress-related factors (SOD2, Catalase, HMOX1, and GPX1).

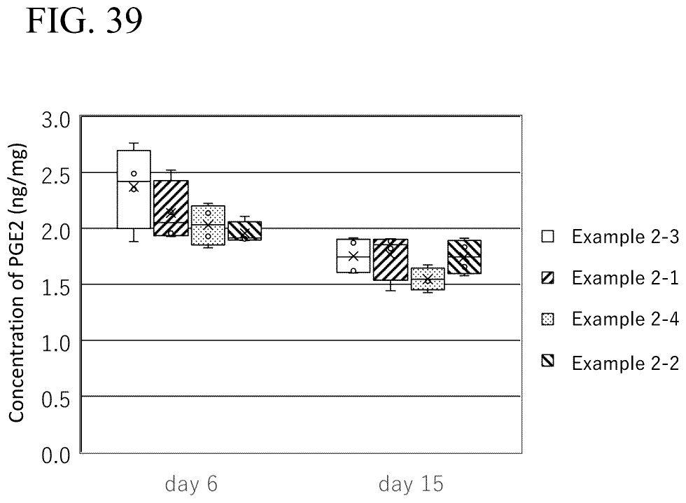

FIG. 39 shows a graph of the concentrations of prostaglandin E2 secreted from the mesenchymal stem cells encapsulated in the hydrogel fibers in Examples 2-1 to 2-4. FIG. 39 shows the prostaglandin E2 concentrations in the total proteins in the culture medium on Day 6 and Day 15 since the start of the culture of the mesenchymal stem cells encapsulated in the hydrogel fibers.

When Examples 2-1 to 2-4 were compared with each other, the prostaglandin E2 concentrations are almost equal regardless of the material used as the base material in the hydrogel fibers. Moreover, the prostaglandin E2 concentrations on Day 15 were not significantly lower than the concentrations on Day 6. It is thus found that the amount of PGE2 secreted is maintained over a relatively long period of time.

Next, the effects on macrophage cell proliferation and activity according to Examples 2-1 to 2-4 are described. First, a macrophage cell line RAW264.7 was induced with lipopolysaccharide (LPS), and 6 hours later, the culture supernatants of the culture medium used in Examples 2-1 to 2-4 were added to RAW264.7. Here, the culture supernatant in each Example is a culture supernatant extracted from the culture medium 24 hours after the start of the culture of the mesenchymal stem cells.

24 hours after the addition of the culture supernatant, the expression levels of M1 macrophage-related factors, M2 macrophage-related factors, and an antioxidant stress-related factor extracted from the macrophage cell line RAW264.7 were measured.

In addition, as Reference Example 2-6, the macrophage cell line RAW264.7 was not induced with lipopolysaccharide (LPS), and the expression levels of M1 macrophage-related factors, M2 macrophage-related factors, and an antioxidant stress-related factor extracted from the macrophage cell line RAW264.7 were measured.

Furthermore, as Reference Example 2-7, 6 hours after the induction of the macrophage cell line RAW264.7 with lipopolysaccharide (LPS), only a serum-free GlutaMAX medium that does not contain FBS and the antibiotic was added. 24 hours after the addition of the GlutaMAX medium, the expression levels of the M1 macrophage-related factors, the M2 macrophage-related factors, and the antioxidant stress-related factor extracted from the macrophage cell line RAW264.7 were measured.

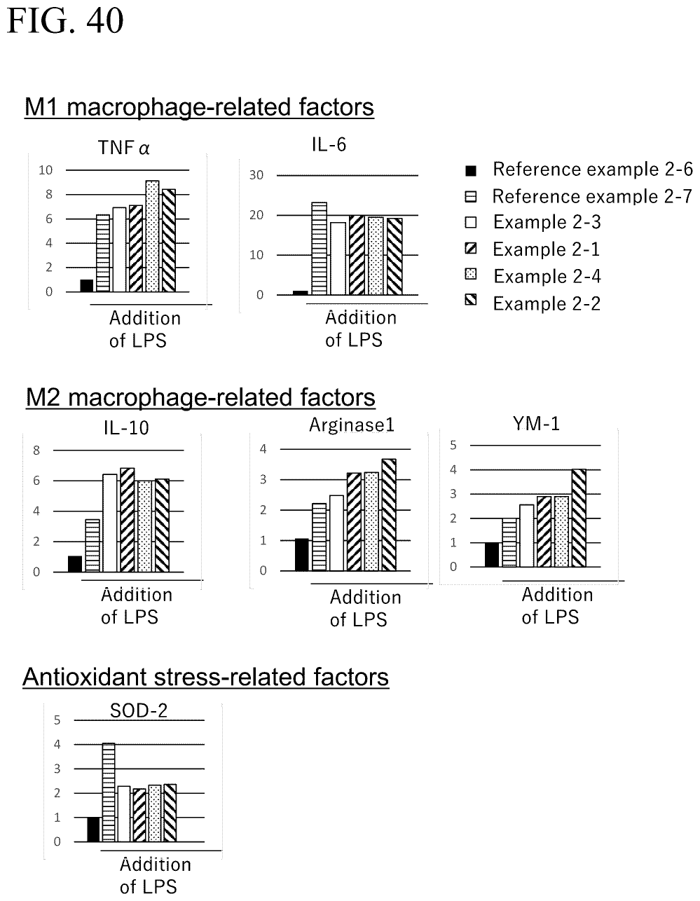

FIG. 40 is a diagram for describing analysis of changes in cell phenotypes in the macrophage cell line RAW264.7 induced with LPS by humoral factors derived from the mesenchymal stem cells in Examples 2-1 to 2-4. The vertical axes represent the expression levels of the various factors in each Example and Reference Example when the expression levels of the various factors in Reference Example 2-6 were normalized as “1”.

FIG. 40 shows the expression levels of TNFα and IL6 as the M1 macrophage-related factors. FIG. 40 also shows the expression levels of IL-10, Arginase 1, and YM-1 as the M2 macrophage-related factors.

The expression levels of the M1 macrophage-related factors in Examples 2-1 to 2-4 were almost equal to those in Reference Example 2-7. On the other hand, the expression levels of IL-10, Arginase 1, and YM-1 showing the M2 phenotype were higher in Examples 2-1 to 2-4 than in Reference Example 2-7. Therefore, it is conceivable that the hydrogel fibers and the culture supernatants thereof in Examples 2-1 to 2-4 are suitable as enhancing agents for the expressions of the M2 macrophage-related factors.

FIG. 40 also shows the expression level of SOD2 as the antioxidant stress-related factor. The expression levels of SOD2 are lower in Examples 2-1 to 2-4 than in Reference Example 2-7 due to the addition of the culture supernatants extracted from the culture medium of the hydrogel fibers. Therefore, it is conceivable that the hydrogel fibers and the culture supernatants thereof in Examples 2-1 to 2-4 can be used as inhibitors of the expression of the antioxidant stress-related factor.

It is conceivable from such results that the hydrogel fibers and the culture supernatants thereof in Examples 2-1 to 2-4 have the effect of suppressing the cell proliferation or activity of macrophage. Therefore, it is conceivable that the hydrogel fibers and the culture supernatants thereof in Examples 2-1 to 2-4 can be used as inhibitors of the cell proliferation or activity of macrophage.

[Chronic Enteritis Model Mice (3)]

### Examples 2-A and 2-B and Reference Examples 2-1, 2-A, and 2-5

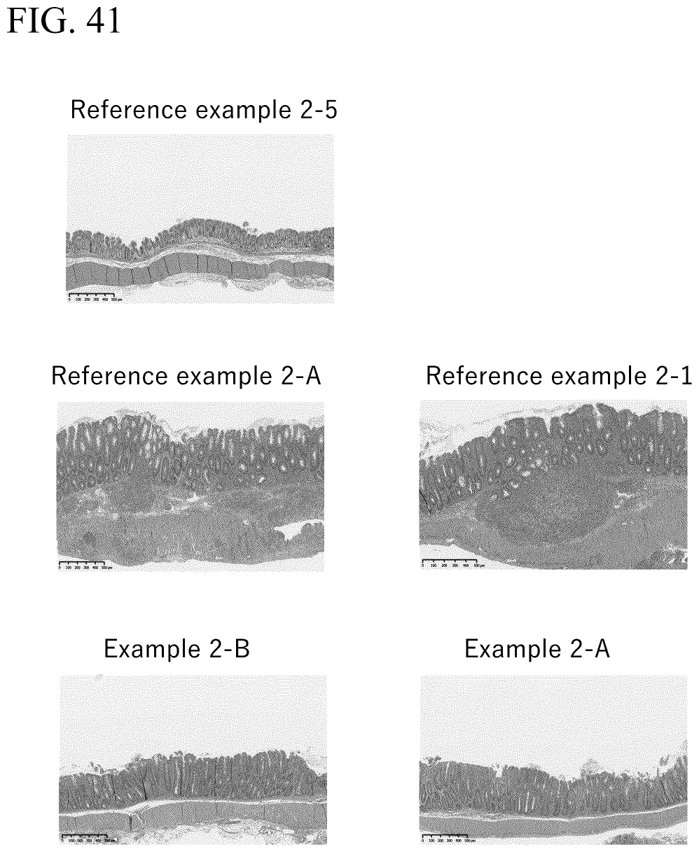

FIG. 41 shows micrographs of histopathological images of large intestines acquired after transplanting the mesenchymal stem cells in Examples 2-A and 2-B and Reference Examples 2-1, 2-A, and 2-5 into chronic enteritis model mice. FIG. 41 shows the histopathological images of the large intestines acquired 47 days after the transplantation. Descriptions of Examples 2-A and 2-B and Reference Examples 2-1, 2-A, and 2-5 are as described above. In Examples 2-A and 2-B, cell infiltration in a submucosa from a muscle layer was decreased, and wall thickening was improved compared to Reference Example 2-A. Marked inflammatory cell infiltration and lymphoid follicle formation were observed in Reference Example 2-1, and improvement was not apparent compared to Reference Example 2-A.

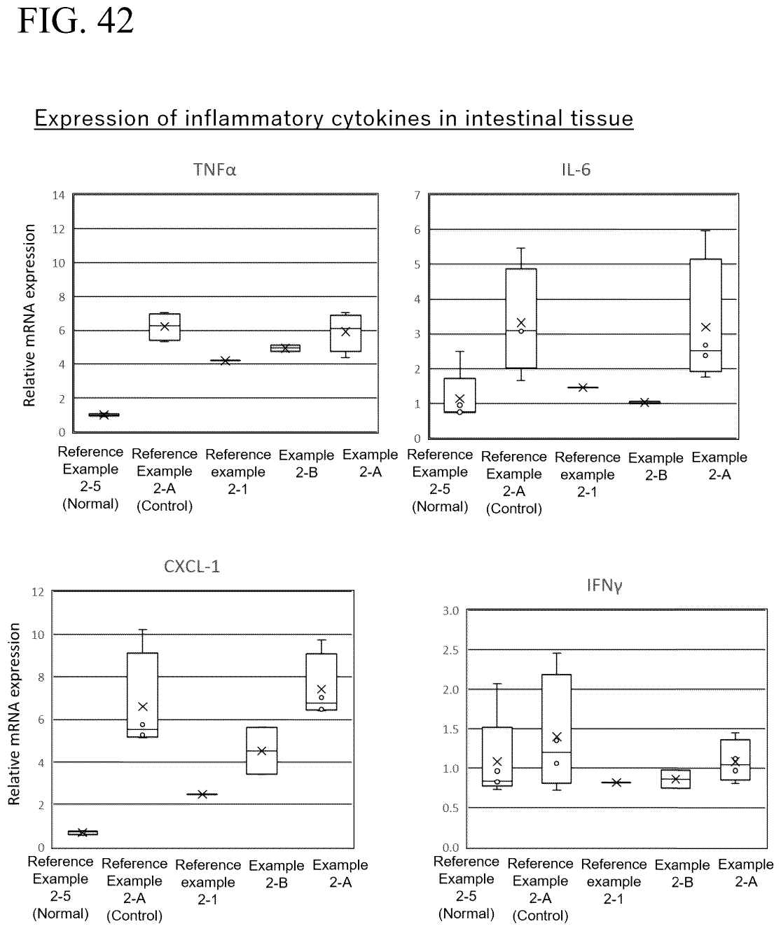

FIG. 42 shows graphs of the expression levels of inflammatory cytokines in intestinal tissues acquired after transplanting the mesenchymal stem cells in Examples 2-A and 2-B and Reference Examples 2-1, 2-A, and 2-5. FIG. 42 shows TNFα, IL-6, CXCL-1, and IFNγ as the inflammatory cytokines in the intestinal tissues.

Referring to FIG. 42, the expressions of the various inflammatory cytokines were suppressed more in Example 2-B than in Reference Example 2-A.

[Hydrogel Fiber Characteristic Analysis (7)]

### Examples 3-1 to 3-3

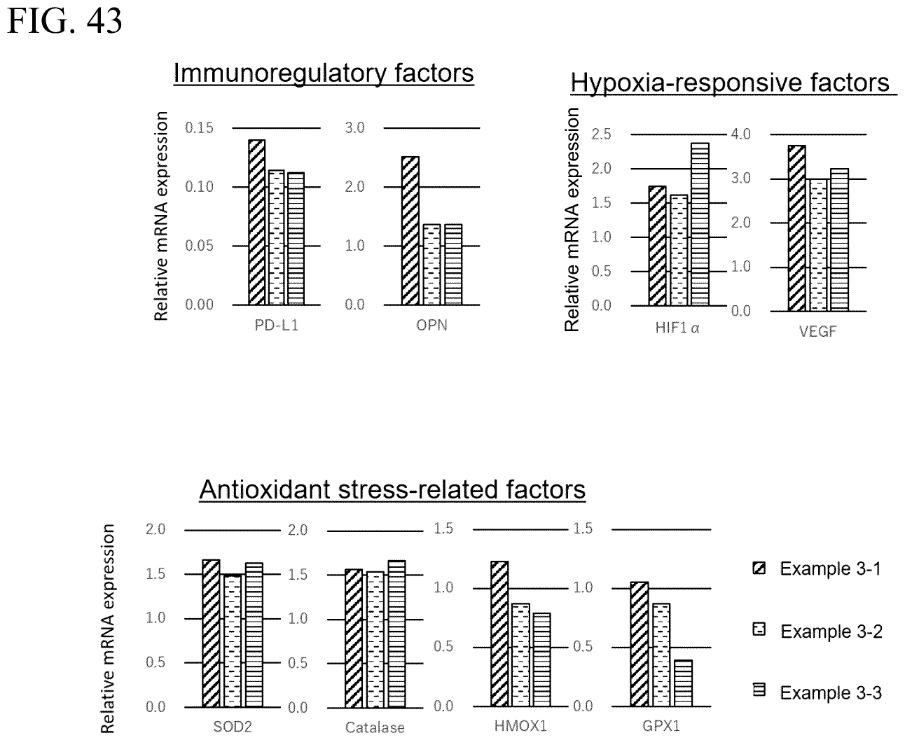

FIG. 43 shows graphs of the results of measuring various expression factors related to the mRNA of the mesenchymal stem cells encapsulated in the hydrogel fibers in Examples 3-1 to 3-3. Here, the descriptions of the hydrogel fibers in Example 3-1 to 3-3 are as described above. In FIG. 43, each Example indicates the results obtained by performing measurement on Day 9 after the preparation of the hydrogel fibers.

FIG. 43 shows the results obtained by conducting additional experiments on factors other than the functional factors shown in FIG. 8. FIG. 43 shows immunoregulatory factors (PD-L1 and OPN), hypoxia-responsive factors (HIF1α and VEGF), and antioxidant stress-related factors (SOD2, Catalase, HMOX1, and GPX1).

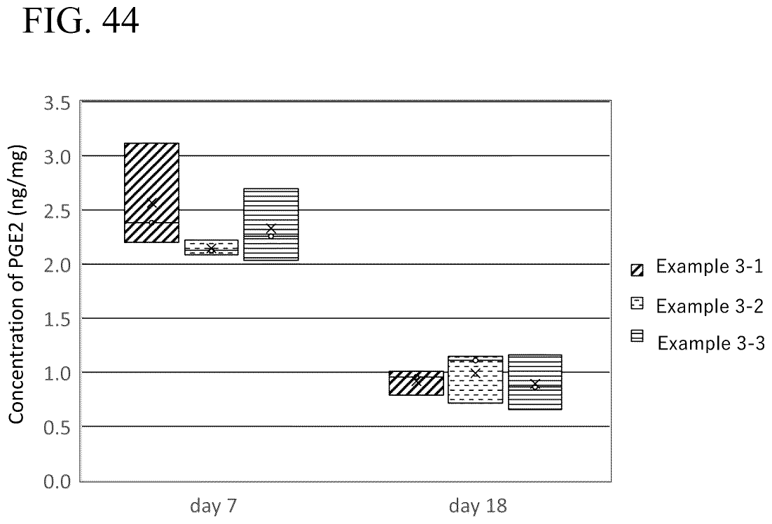

FIG. 44 shows a graph of the concentrations of prostaglandin E2 secreted from the mesenchymal stem cells encapsulated in the hydrogel fibers in Examples 3-1 to 3-3. FIG. 44 shows the prostaglandin E2 concentrations in the culture medium on Day 7 and Day 18 since the start of the culture of the mesenchymal stem cells encapsulated in the hydrogel fibers.

When Examples 3-1 to 3-3 were compared with each other, the prostaglandin E2 concentrations are almost equal regardless of the material used as the base material in the hydrogel fibers.

Next, the effects on macrophage cell proliferation and activity according to Examples 3-1 to 3-3 are described. First, a macrophage cell line RAW264.7 was induced with lipopolysaccharide (LPS), and 6 hours later, the culture supernatants of the culture medium used in Examples 3-1 to 3-3 were added to RAW264.7. Here, the culture supernatant in each Example is a culture supernatant extracted from the culture medium 24 hours after the start of the culture of the mesenchymal stem cells.

24 hours after the addition of the culture supernatant, the expression levels of M1 macrophage-related factors, M2 macrophage-related factors, and an antioxidant stress-related factor extracted from the macrophage cell line RAW264.7 were measured.

In addition, as Reference Example 3-3, the macrophage cell line RAW264.7 was not induced with lipopolysaccharide (LPS), and the expression levels of M1 macrophage-related factors, M2 macrophage-related factors, and an antioxidant stress-related factor extracted from the macrophage cell line RAW264.7 were measured.

Furthermore, as Reference Example 3-4, 6 hours after the induction of the macrophage cell line RAW264.7 with lipopolysaccharide (LPS), only a serum-free GlutaMAX medium that does not contain FBS and an antibiotic was added. 24 hours after the addition of the GlutaMAX medium, the expression levels of the M1 macrophage-related factors, the M2 macrophage-related factors, and the antioxidant stress-related factor extracted from the macrophage cell line RAW264.7 were measured.

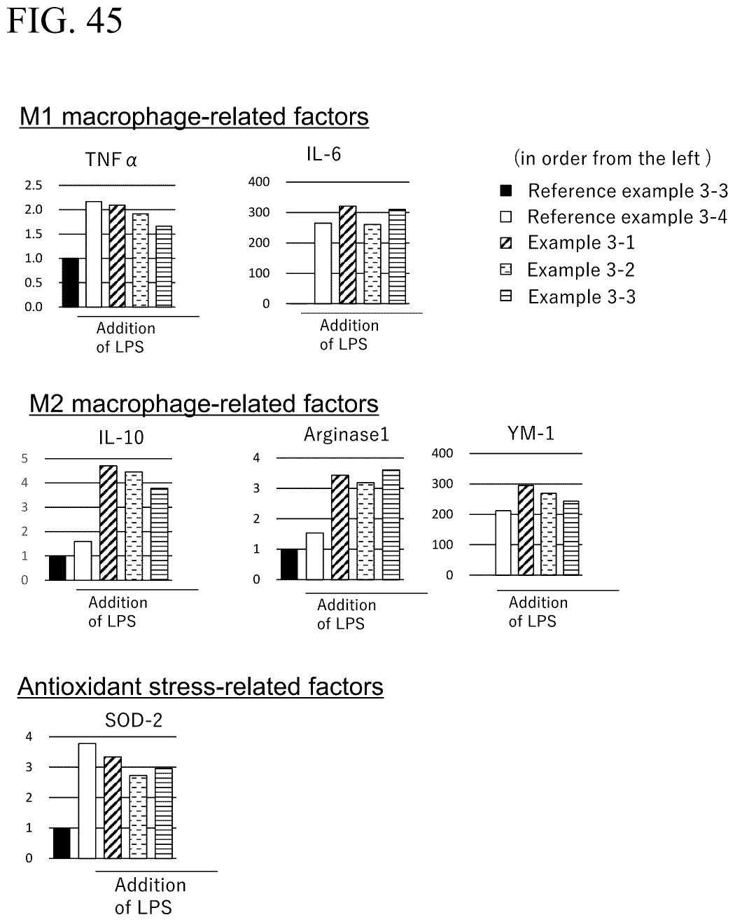

FIG. 45 is a diagram for describing analysis of changes in cell phenotypes in the macrophage cell line RAW264.7 induced with LPS by humoral factors derived from the mesenchymal stem cells in Examples 3-1 to 3-3. The vertical axes represent the expression levels of the various factors in each Example and Reference Example when the expression levels of the various factors in Reference Example 3-3 were normalized as “1”.

The expression levels of the M1 macrophage-related factors in Examples 3-1 to 3-3 were almost equal to those in Reference Example 3-4. On the other hand, the expression levels of IL-10, Arginase 1, and YM-1 showing the M2 phenotype were higher in Examples 3-1 to 3-3 than in Reference Example 3-4. Therefore, it is conceivable that the hydrogel fibers and the culture supernatants thereof in Examples 3-1 to 3-3 are suitable as enhancing agents for the expressions of the M2 macrophage-related factors.

FIG. 45 also shows the expression level of SOD2 as the antioxidant stress-related factor. The expression levels of SOD2 are lower in Examples 3-1 to 3-3 than in Reference Example 3-4 due to the addition of the culture supernatants extracted from the culture medium of the hydrogel fibers. Therefore, it is conceivable that the hydrogel fibers and the culture supernatants thereof in Examples 3-1 to 3-3 can be used as inhibitors of the expression of the antioxidant stress-related factor.

It is conceivable from such results that the hydrogel fibers and the culture supernatants thereof in Examples 3-1 to 3-3 have the effect of suppressing the cell proliferation or activity of macrophage. Therefore, it is conceivable that the hydrogel fibers and the culture supernatants thereof in Examples 3-1 to 3-3 can be used as inhibitors of the cell proliferation or activity of macrophage.

[Chronic Enteritis Model Mice (4)]

### Examples 3-1 to 3-3 and Reference Examples 3-1 and 3-2

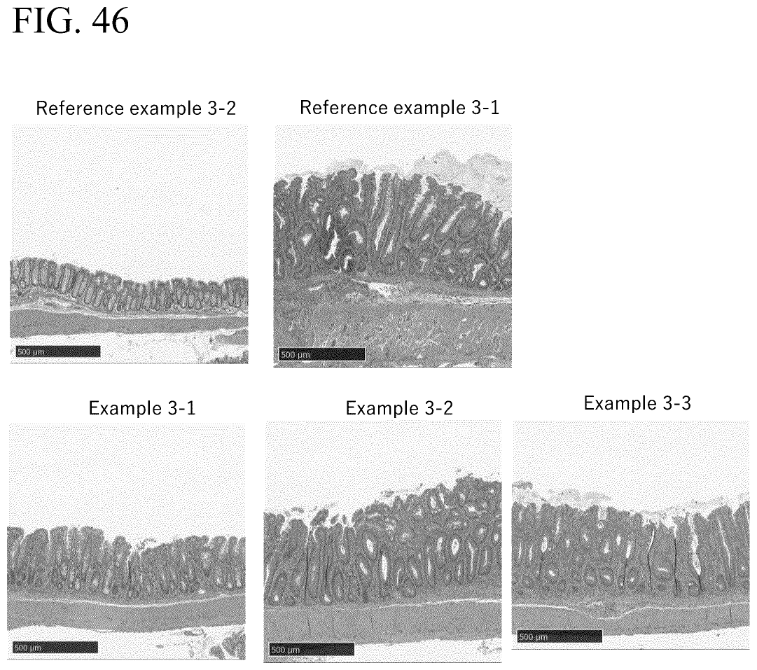

FIG. 46 shows micrographs of histopathological images of large intestines acquired after transplanting the mesenchymal stem cells in Examples 3-1 to 3-3 and Reference Examples 3-1 and 3-2 into chronic enteritis model mice. FIG. 46 shows the histopathological images of the large intestines acquired 26 days after the transplantation. Descriptions of Examples 3-1 to 3-3 and Reference Examples 3-1 and 3-2 are as described above. In Example 3-1 to 3-3, cell infiltration in a submucosa from a muscle layer was decreased compared to Reference Example 3-1, and particularly in Example 3-1, wall thickening due to cell infiltration in lamina propria was also improved.

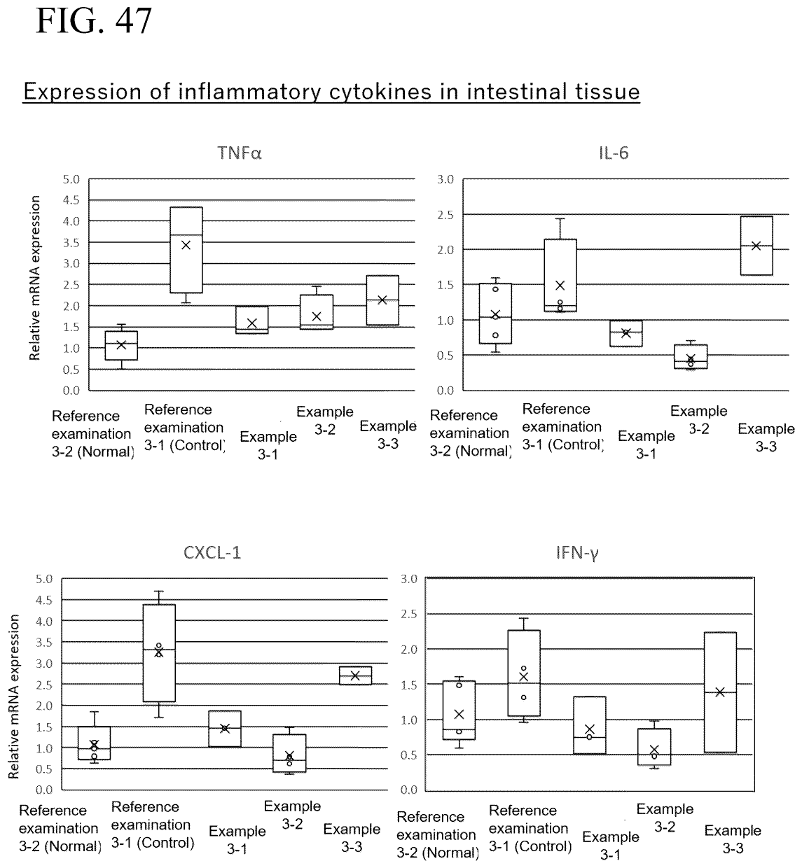

FIG. 47 shows graphs of the expression levels of inflammatory cytokines in intestinal tissues acquired after transplanting the mesenchymal stem cells in Examples 3-1 to 3-3 and Reference Examples 3-1 and 3-2. FIG. 47 shows TNFα, IL-6, CXCL-1, and IFNγ as the inflammatory cytokines in the intestinal tissues.

Referring to FIG. 47, the expressions of the various inflammatory cytokines was suppressed more in Examples 3-1 and 3-2 than in Reference Example 3-1.

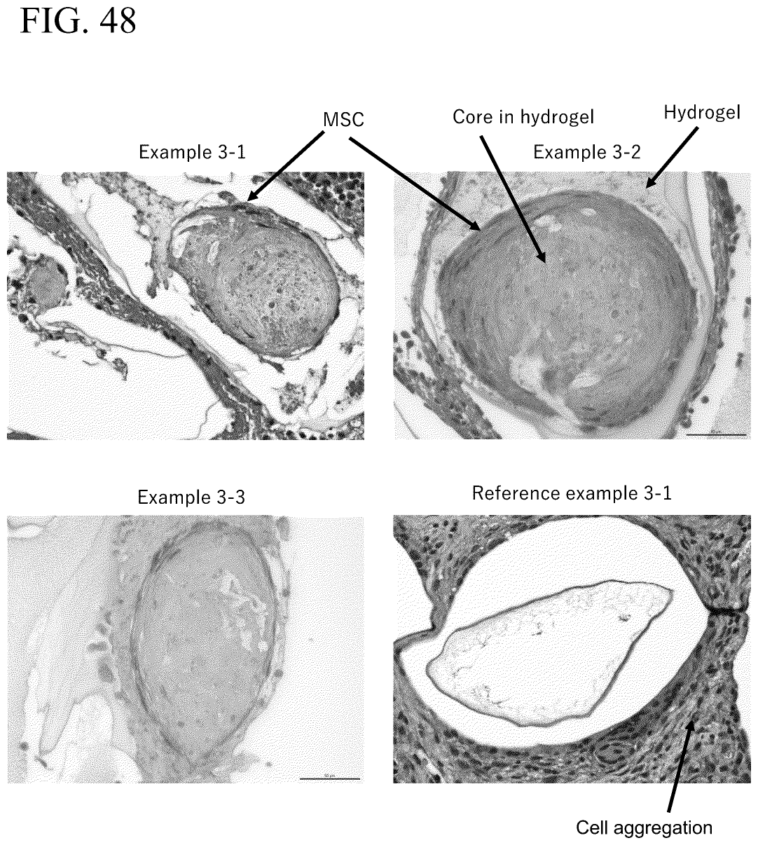

FIG. 48 shows micrographs of the surroundings of hydrogel structures in Examples 3-1 to 3-3 and Reference Example 3-1 that have been resected from peritoneal cavities after the transplantation of the hydrogel structures. FIG. 48 shows the micrographs acquired 26 days after the transplantation. Viable mesenchymal stem cells remained on the surface layer of the base material (core) inside the hydrogel structure, and morphology of the cells was similar to that before the administration. Marked cell aggregation was observed around the empty hydrogel fiber that did not encapsulate the mesenchymal stem cells (Reference Example 3-1), whereas the cell aggregation was small around the hydrogel fibers that encapsulated the mesenchymal stem cells (Examples 3-1 to 3-3).

[Hydrogel Fiber Characteristic Analysis (8)]

### Examples 4-1 and 4-2

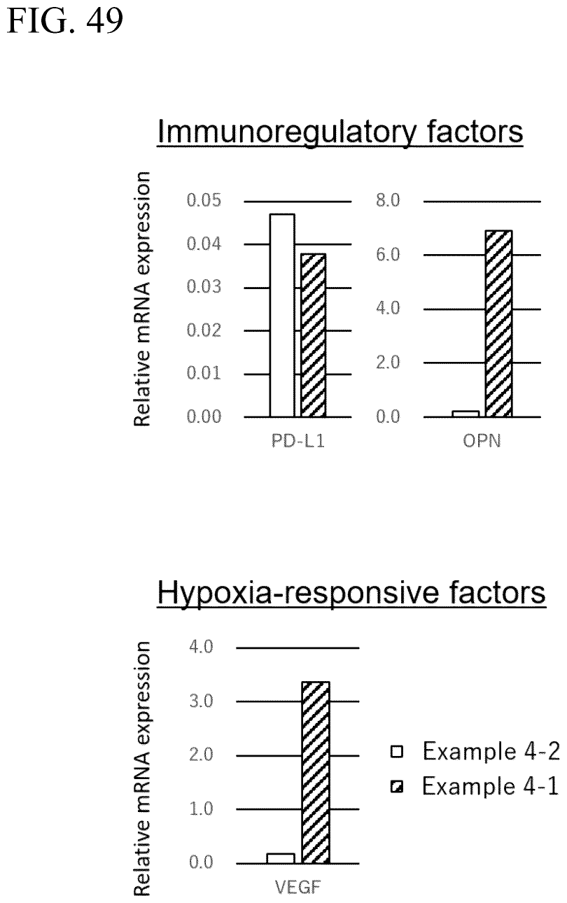

FIG. 49 shows graphs of the results of measuring various expression factors related to the mRNA of the mesenchymal stem cells encapsulated in the hydrogel fibers in Examples 4-1 and 4-2. Here, the descriptions of the hydrogel fibers in Example 4-1 and 4-2 are as described above. FIG. 49 shows the measurement results obtained on Day 20 since the start of the culture.

FIG. 49 shows the results obtained by conducting additional experiments on factors other than the functional factors shown in FIG. 10. FIG. 49 shows immunoregulatory factors (PD-L1 and OPN) and a hypoxia-responsive factor (VEGF).

[Hydrogel Fiber Characteristic Analysis (9)]

### Example 5-1

A hydrogel fiber which is a hydrogel structure according to Example 5-1 will be described. The hydrogel fiber according to Example 5-1 was produced in the same manner as in Example 3-1, except for the tissue from which the mesenchymal stem cells were derived and the number of the cells encapsulated in the hydrogel fiber (cell density). Thus, the core solution used in Example 5-1 as the base material during the hydrogel fiber production contains an atelocollagen solution.

The cells used in Example 5-1 are human bone marrow-derived mesenchymal stem cells. Furthermore, in Example 5-1, the density of the cells in the cell suspension during the hydrogel fiber production (initial cell density) was about 5×10 7 cells/mL. As the collagen solution, 3% Koken Atelocollagen Implant (manufactured by KOKEN CO., LTD., #1333) was used. The final concentration of the collagen solution is 4 mg/mL.

### Example 5-2

A hydrogel fiber according to Example 5-2 was produced in the same manner as in Example 5-1, except for the core solution used as the base material during the hydrogel fiber production. Thus, the cells used in Example 5-2 are human bone marrow-derived mesenchymal stem cells.

The core solution in Example 5-2 is a medium. The medium is obtained by adding fetal bovine serum (FBS) and an antibiotic to Dulbecco's Modified Eagle's Medium (high glucose) (manufactured by Sigma-Aldrich, Inc.: D6429).

Next, the cases of immersing and culturing the human bone marrow-derived mesenchymal stem cells encapsulated in the hydrogel fibers in Examples 5-1 and 5-2 in the medium together with the fibers were compared with the case of performing 2-dimensional culture of the mesenchymal stem cells without encapsulating the mesenchymal stem cells in the hydrogel fibers (Reference Example 5-1). Specifically, the amount of various humoral factors secreted into the medium and various expression factors related to mRNA were measured.

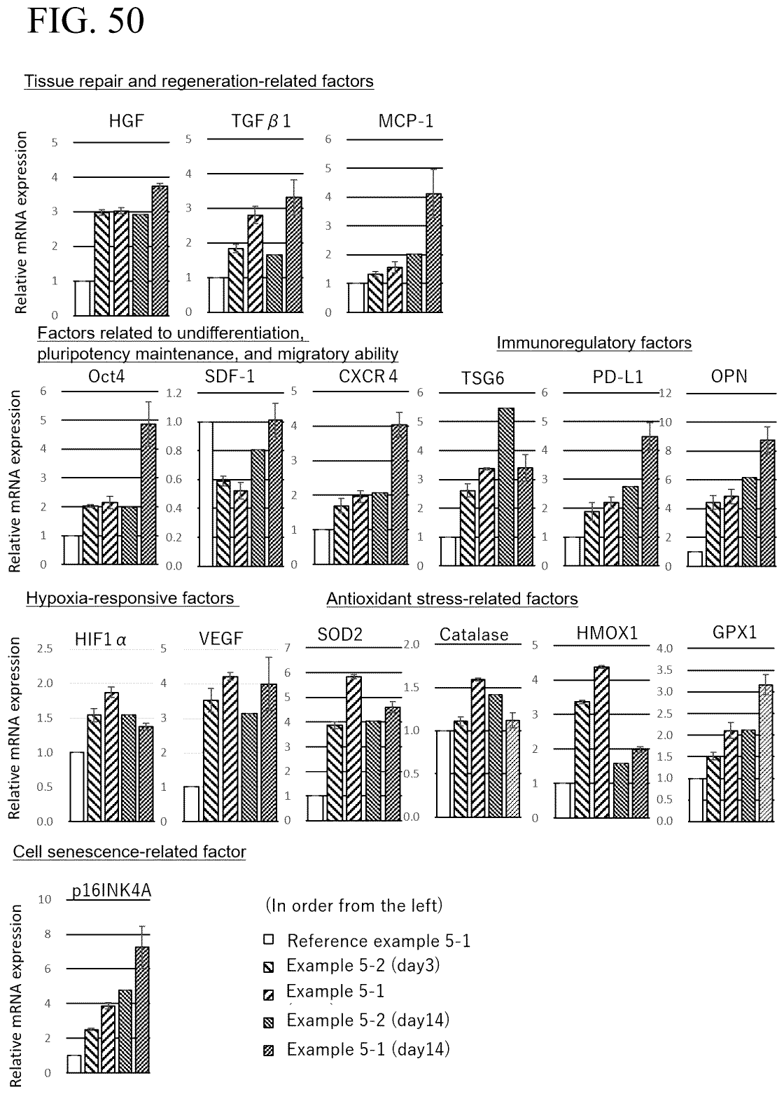

FIG. 50 shows graphs of the results of measuring various expression factors related to the mRNA of mesenchymal stem cells encapsulated in hydrogels in Examples 5-1 and 5-2. The vertical axes in FIG. 50 represent the ratios obtained when the value for the mesenchymal stem cells subjected to the 2-dimensional culture (Reference Example 5-1) was normalized as “1”. FIG. 50 shows the measurement results obtained when 3 days have passed and when 14 days have passed since the start of the culture.

FIG. 50 shows tissue repair and regeneration-related factors (HGF, TGEβ, and MCP-1), undifferentiation/pluripotency maintenance/cellular motility-related factors (Oct-4, SDF-1, and CXCR4), immunoregulatory factors (TSG6, PD-L1, and OPN), hypoxia-responsive factors (HIF1α and VEGF), antioxidant stress-related factors (SOD2, Catalase, HMOX1, and GPX1), and a cell senescence-related factor and tumor suppressor gene (p16INK4A).

The expression levels of the factors in the mesenchymal stem cells encapsulated in the hydrogel fibers in Examples 5-1 and 5-2 were equal to or higher than the expression levels of the functional factors in the 2-dimensional culture (Reference Example 5-1), except for SDF-1. It is thus found that the encapsulation of the human bone marrow-derived mesenchymal stem cells in the hydrogel can contribute to the increase of a number of expression factors related to the mRNA.

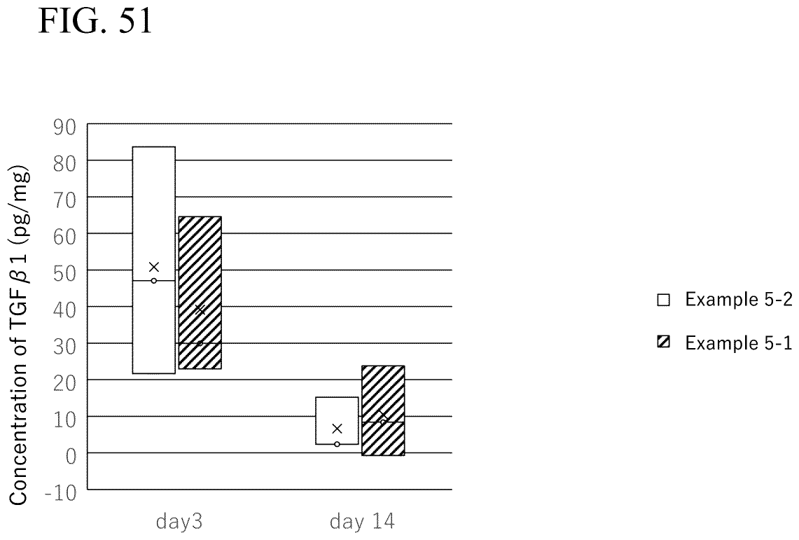

FIG. 51 shows a graph of the results of measuring a humoral factor (TGF-β1) secreted from the mesenchymal stem cells encapsulated in the hydrogels in Examples 5-1 and 5-2. The vertical axis in FIG. 51 represents the concentration of TGF-β1 in the total proteins in the medium. When the day on which the hydrogel fibers were prepared was set to Day 0, TGF-β1 was measured on Day 3 and Day 14.

In each of Examples 5-1 and 5-2, the experiment was conducted with 3 samples. The central value in the longitudinal direction in each rectangle is the mean value of the results of the experiment conducted with the 3 hydrogel fibers. The length of each rectangle in the longitudinal direction represents the standard deviation (dispersion) of the results of the experiment conducted with the 3 hydrogel fibers.

The amounts of TGF-β1 secreted were nearly equal in both Examples 5-1 and 5-2. In both Examples 5-1 and 5-2, the amounts of TGF-β1 secreted decreased as the number of days (culture period) increased since the preparation of the hydrogel fibers.

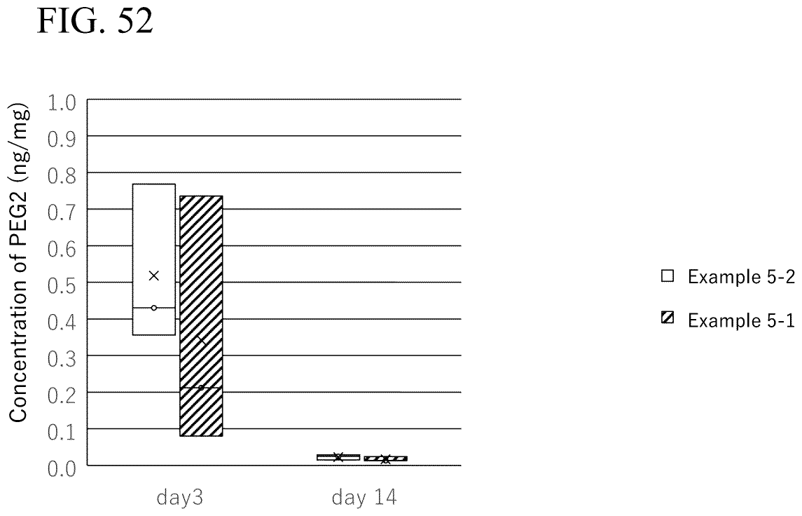

FIG. 52 shows a graph of the results of measuring a humoral factor (prostaglandin E2; PGE2) secreted from the mesenchymal stem cells encapsulated in the hydrogels in Examples 5-1 and 5-2.

The vertical axis in FIG. 52 represents the concentration of PGE2 in the total proteins in the medium. In each of Examples 5-1 and 5-2, the experiment was conducted with 3 hydrogel fibers. The central value in the longitudinal direction in each rectangle is the mean value of the results of the experiment conducted with the 3 hydrogel fibers. The length of each rectangle in the longitudinal direction represents the standard deviation (dispersion) of the results of the experiment conducted with the 3 hydrogel fibers.

When Example 5-1 and Example 5-2 were compared with each other, the amounts of PGE2 secreted were nearly equal to each other.

According to FIGS. 51 and 52, it was found that the factors TGF-β1 and PGE2 were increased even in a case where the bone marrow-derived mesenchymal stem cells were encapsulated in the hydrogel. Therefore, the bone marrow-derived mesenchymal stem cells are expected to produce the same preferred results as those obtained with the umbilical cord-derived mesenchymal stem cells. Thus, the hydrogel structure of the present invention is expected to exhibit the preferred effect regardless of the tissue from which the mesenchymal stem cells are derived.

[Hydrogel Fiber Characteristic Analysis (10)]

### Example 6-1

Next, a hydrogel fiber which is a hydrogel structure according to Example 6-1 will be described. The hydrogel fiber according to Example 6-1 was produced by the same method as that in Example 1-2. Thus, the core solution used in Example 6-1 as the base material during the hydrogel fiber production is a medium. The medium is obtained by adding fetal bovine serum (FBS) and an antibiotic to a GlutaMAX medium (manufactured by Thermo Fisher Scientific Inc.: Cat No. 32571-036). Furthermore, in Example 6-1, the density of the human umbilical cord-derived mesenchymal stem cells in the cell suspension during the hydrogel fiber production (initial cell density) was about 1×10⁸ cells/mL (refer to Table 3).

### Example 6-2

A hydrogel fiber which is a hydrogel structure according to Example 6-2 was produced in the same manner as in Example 6-1, except for the number of cells encapsulated in the hydrogel fiber (cell density). In Example 6-2, the density of the human umbilical cord-derived mesenchymal stem cells in the cell suspension during the hydrogel fiber production (initial cell density) was about 5×10⁷ cells/mL (refer to Table 3).

### Example 6-3

A hydrogel fiber which is a hydrogel structure according to Example 6-3 was produced in the same manner as in Example 6-1, except for the number of cells encapsulated in the hydrogel fiber (cell density). In Example 6-3, the density of the human umbilical cord-derived mesenchymal stem cells in the cell suspension during the hydrogel fiber production (initial cell density) was about 1×10⁷ cells/mL (refer to Table 3).

### Example 6-4

A hydrogel fiber which is a hydrogel structure according to Example 6-4 was produced in the same manner as in Example 6-1, except for the core solution used as the base material during the hydrogel fiber preparation. The core solution in Example 6-4 contains an atelocollagen solution (refer to Table 3). As the collagen solution, 3% Koken Atelocollagen Implant (manufactured by KOKEN CO., LTD., #1333) was used. The final concentration of the collagen solution is 4 mg/mL.

### Example 6-5

A hydrogel fiber which is a hydrogel structure according to Example 6-5 was produced in the same manner as in Example 6-4, except for the number of cells encapsulated in the hydrogel fiber (cell density). In Example 6-4, the density of the human umbilical cord-derived mesenchymal stem cells in the cell suspension during the hydrogel fiber production (initial cell density) was about 5×10⁷ cells/mL (refer to Table 3).

### Example 6-6

A hydrogel fiber which is a hydrogel structure according to Example 6-6 was produced in the same manner as in Example 6-4, except for the number of cells encapsulated in the hydrogel fiber (cell density). In Example 6-6, the density of the human umbilical cord-derived mesenchymal stem cells in the cell suspension during the hydrogel fiber production (initial cell density) was about 1×10⁷ cells/mL (refer to Table 3).

TABLE 3

| | | Tissue of origin | Cell density | | |
|---|---|---|---|---|---|
| | Cells | of cells | (cells/mL) | Base Material | Hydrogel |
| Example 6-1 | MSC | Umbilical cord | 1 × 10⁸ | Medium | Calcium alginate |
| Example 6-2 | MSC | Umbilical cord | 5 × 10⁷ | Medium | Calcium alginate |
| Example 6-3 | MSC | Umbilical cord | 1 × 10⁷ | Medium | Calcium alginate |
| Example 6-4 | MSC | Umbilical cord | 1 × 10⁸ | with atelocollagen | Calcium alginate |
| Example 6-5 | MSC | Umbilical cord | 5 × 10⁷ | with atelocollagen | Calcium alginate |
| Example 6-6 | MSC | Umbilical cord | 1 × 10⁷ | with atelocollagen | Calcium alginate |
| Reference | *MSC (2D culture) | | | | |
| example 6-1 | | | | | |

Next, the cases of immersing and culturing the human umbilical cord-derived mesenchymal stem cells encapsulated in the hydrogel fibers in Examples 6-1 to 6-6 in the medium together with the fibers were compared with the case of performing 2-dimensional culture of the mesenchymal stem cells without encapsulating the mesenchymal stem cells in the hydrogel fibers (Reference Example 6-1). Specifically, the amounts of various humoral factors secreted into the medium, various expression factors related to mRNA, and the like were measured.

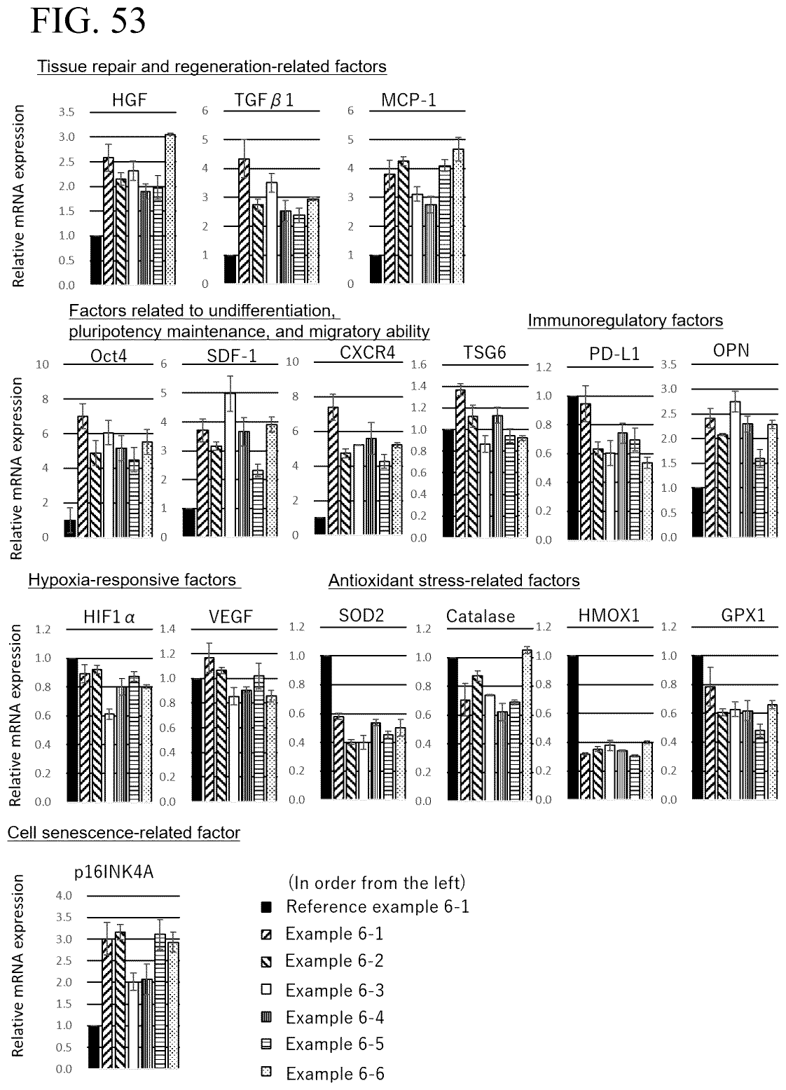

FIG. 53 shows graphs of the results of measuring various expression factors related to the mRNA of the mesenchymal stem cells encapsulated in the hydrogels in Examples 6-1 and 6-6. The vertical axes in FIG. 53 represent the ratios obtained when the value for the mesenchymal stem cells subjected to the 2-dimensional culture (Reference Example 6-1) was normalized as “1”. FIG. 53 shows the measurement results obtained when 16 days have passed since the start of the culture.

FIG. 53 shows tissue repair and regeneration-related factors (HGF, TGEβ, and MCP-1), undifferentiation/pluripotency maintenance/cellular motility-related factors (Oct-4, SDF-1, and CXCR4), immunoregulatory factors (TSG6, PD-L1, and OPN), hypoxia-responsive factors (HIF1α and VEGF), antioxidant stress-related factors (SOD2, Catalase, HMOX1, and GPX1), and a cell senescence-related factor and tumor suppressor gene (p16INK4A).

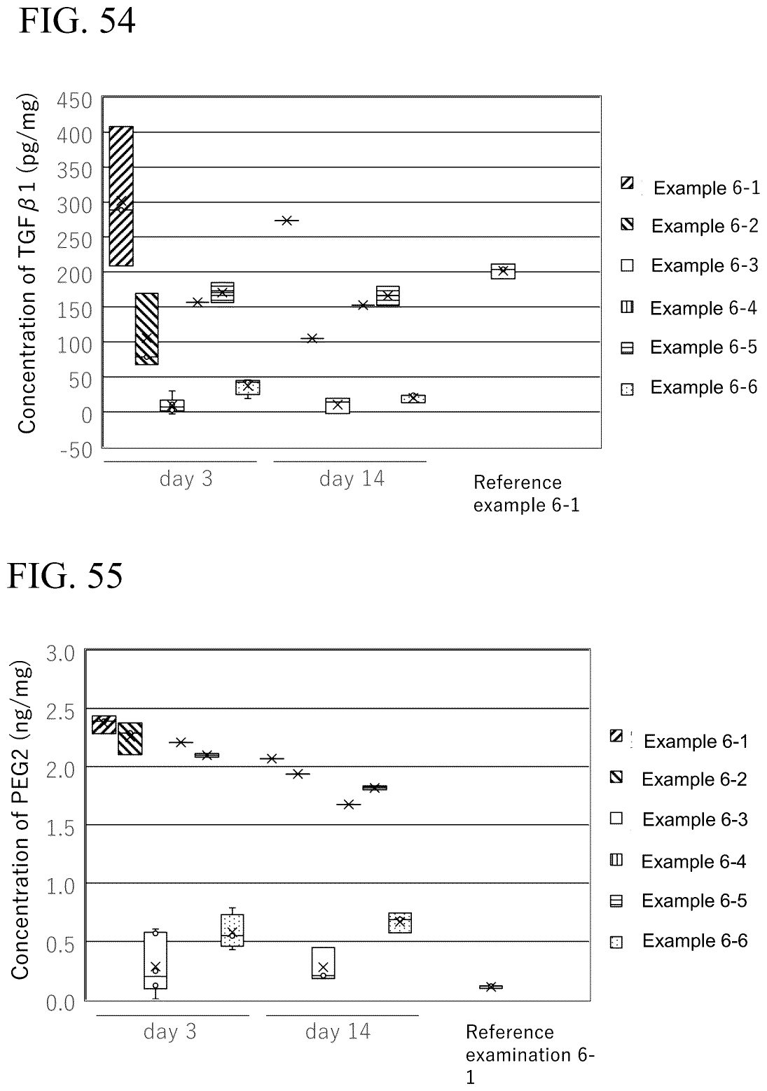

FIG. 54 shows a graph of the results of measuring a humoral factor (TGF-β1) secreted from the mesenchymal stem cells encapsulated in the hydrogels in Examples 6-1 and 6-6. The vertical axis in FIG. 54 represents the concentration of TGF-β1 in the total proteins in the medium. When the day on which the hydrogel fibers were prepared was set to Day 0, TGF-β1 was measured on Day 3 and Day 14. In FIG. 54, the results for Examples 6-1, 6-2, 6-3, 6-4, 6-5, and 6-6 are shown in order from the left on each of Day 3 and Day 14.

The central value in the longitudinal direction in each rectangle is the mean value of the results of the experiment conducted with a plurality of hydrogel fibers. The length of each rectangle in the longitudinal direction represents the standard deviation (dispersion) of the results of the experiment conducted with the plurality of hydrogel fibers.

It is understood from FIG. 54 that the amount of TGF-β1 secreted generally increases as the initial cell density increases. Thus, the initial cell density is preferably as high as possible. However, the amounts of TGF-β1 secreted were nearly equal in Examples 6-4 and 6-6. In addition, the amounts of TGF-β1 secreted on Day 3 and Day 14 since the hydrogel fiber preparation were nearly equal in each Example. Therefore, the amount of TGF-β1 secreted is maintained over a long period of time.

FIG. 55 shows a graph of the results of measuring a humoral factor (prostaglandin E2; PGE2) secreted from the mesenchymal stem cells encapsulated in the hydrogels in Examples 6-1 and 6-6.

The vertical axis in FIG. 55 represents the concentration of PGE2 in the total proteins in the medium. In FIG. 55, the results for Examples 6-1, 6-2, 6-3, 6-4, 6-5, and 6-6 are shown in order from the left on each of Day 3 and Day 14. The central value in the longitudinal direction in each rectangle is the mean value of the results of the experiment conducted with a plurality of hydrogel fibers. The length of each rectangle in the longitudinal direction represents the standard deviation (dispersion) of the results of the experiment conducted with the plurality of hydrogel fibers.

The amounts of PGE2 secreted in Examples 6-1, 6-2, 6-4, and 6-5 in which the initial cell densities were high were greater than the amounts of PGE2 secreted in the rest of the examples, Examples 6-3 and 6-6. In addition, the amounts of PGE2 secreted on Day 3 and Day 14 since the hydrogel fiber preparation were nearly equal in each Example. Therefore, it is conceivable that the amount of PGE2 secreted is maintained over a long period of time.

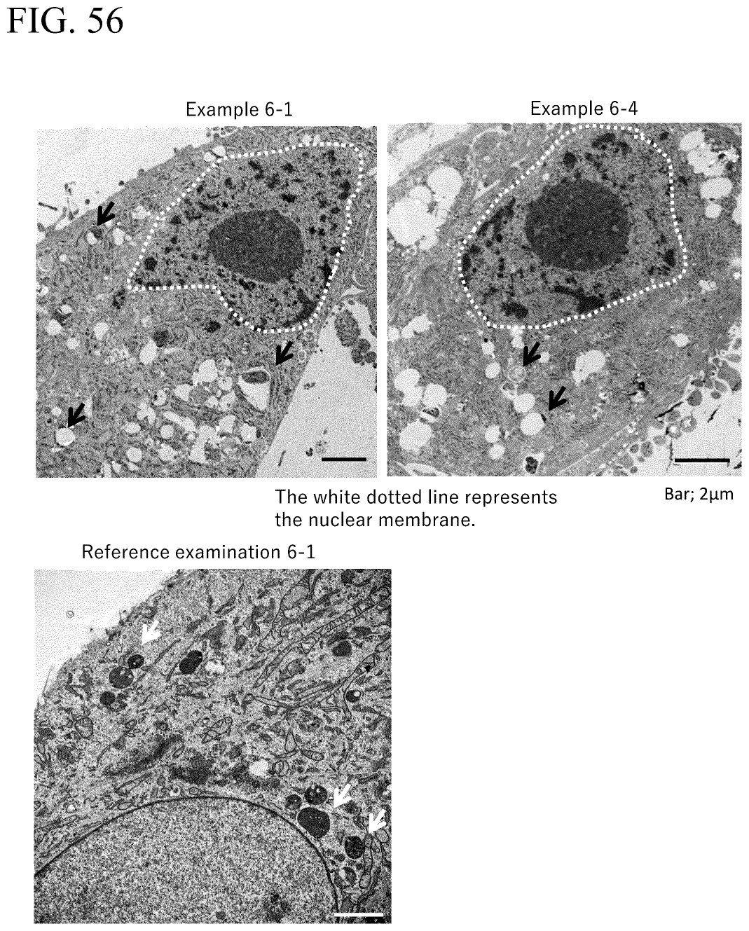

FIG. 56 is a diagram showing images of autophagy observed under a transmission electron microscope, which are related to microstructures of the mesenchymal stem cells in Examples 6-1 and 6-4 and Reference Example 6-1. FIG. 56 shows the images of the autophagy observed on Day 14 since the hydrogel fiber preparation.

In Reference Example 6-1, degenerated mitochondria (Mit) are observed to be scattered in the cytoplasm of the mesenchymal stem cell (refer to the white arrows in the figure). In Examples 6-1 and 6-4, many images were observed in which degenerated mitochondria were being processed, and the autophagy progressed (refer to the black arrows in the figure). Furthermore, a tendency for the endoplasmic reticulum structure to be maintained was observed in Examples 6-1 and 6-4.

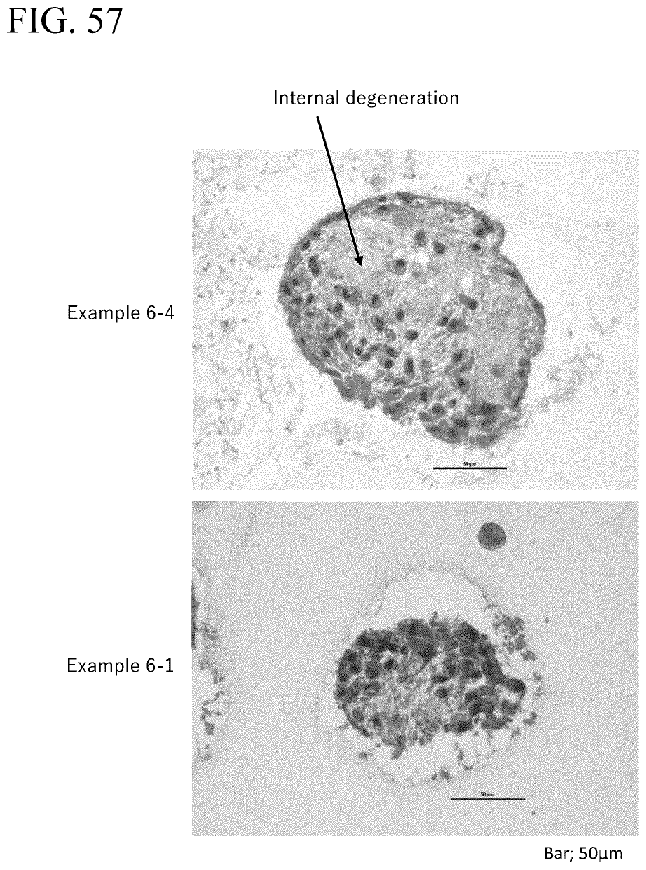

FIG. 57 shows magnified photographs of H&E-stained cross-sectional images of the mesenchymal stem cells (spheroids) within the hydrogel fibers in Examples 6-1 and 6-4. From FIG. 57, it is found that the mesenchymal stem cells are aggregated in the hydrogel fiber, thus forming the spheroids. The spheroid had a core as a central portion formed of degenerated cells and/or atelocollagen contained in the core solution and a double- or triple-layer of viable cells on the outside of the core.

The diameter of the spheroid according to Example 6-4 is larger than the diameter of the spheroid according to Example 6-1. In Example 6-4, the volume of the atelocollagen functioning as the scaffold, that is, the atelocollagen (base material) used during the hydrogel fiber production is considered to increase the spheroid diameter. Furthermore, it is considered that, in Example 6-4, the boundary between the atelocollagen (base material) scaffold and the cells is relatively clear, and the site of the scaffold contains an eosinophilic, unstructured region.

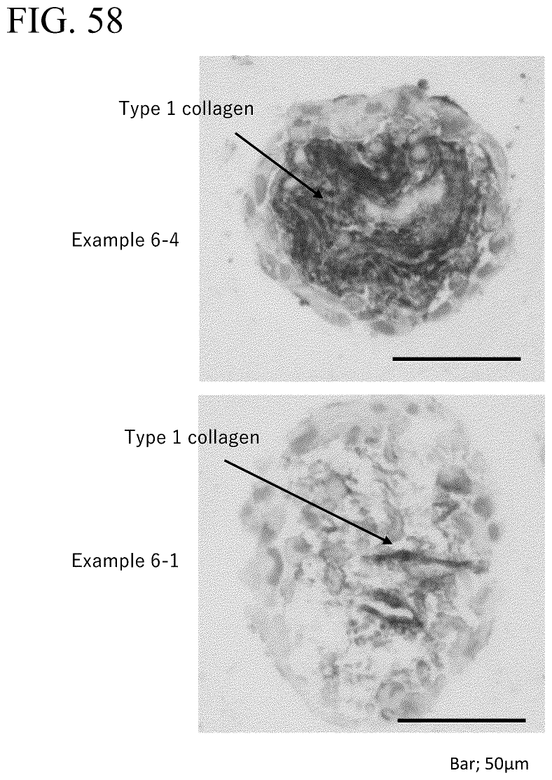

FIG. 58 shows magnified photographs of the mesenchymal stem cells (spheroids) within the hydrogel fibers in Examples 6-1 and 6-4. In Example 6-4, the atelocollagen encapsulated in the hydrogel as the extracellular matrix is localized inside the spheroid. Such atelocollagen exists almost entirely within the spheroid.

In Example 6-1, no collagen is encapsulated in the hydrogel during the hydrogel structure production. However, the spheroid within the hydrogel fiber according to Example 6-1 contained localized type I collagen. It is considered that the type I collagen was obtained by the degeneration of the mesenchymal stem cells per se, or from the extracellular matrix secreted from the mesenchymal stem cells.

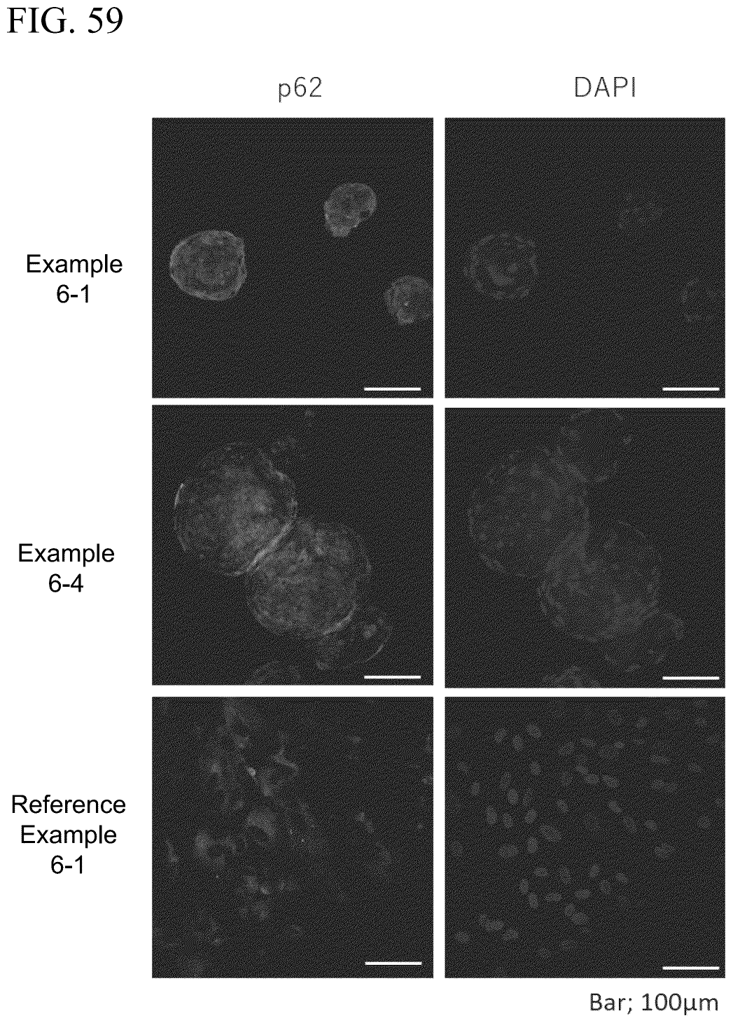

FIG. 59 shows micrographs showing aspects of the expression of an autophagy-related factor p62 in the mesenchymal stem cells (spheroids) within the hydrogel fibers in Examples 6-1 and 6-4. FIG. 59 shows the results of analyzing the p62 expression in the spheroids within the hydrogel fibers on Day 14 since the hydrogel fiber preparation or in the cells subjected to the 2-dimensional culture.

The images on the left side in FIG. 59 are obtained by performing immunofluorescence cell staining using a primary antibody against p62 (anti-p62 (SQSTM1) polyclonal antibody, manufactured by MBL International Corporation, No. PM045) and a fluorescently labeled secondary antibody (Fluoro-conjugated Goat anti-Rabbit-IgG antibody, manufactured by Merck KGaA, AP187F) and performing observation with a confocal laser scanning microscope. The part in which green fluorescence is detected indicates the presence of the autophagy-related factor p62. The images on the right side in FIG. 59 are the results of DAPI staining, and the blue fluorescent part indicates the site of the nucleus (DNA) of a viable cell.

In both Examples 6-1 and 6-4, p62 is expressed mainly in viable cells in the vicinity of the surfaces of the spheroids. Also in Reference Example 6-1, p62 is expressed in viable cells. However, the p62 expression on the surfaces of the spheroids in Examples 6-1 and 6-4 is stronger than the p62 expression in Reference Example 6-1.

This indicates that the viable cells are localized on the surface of the spheroid within the hydrogel structure, and autophagy is induced in these viable cells. It is considered that such promotion of autophagy enables the long-term survival of the mesenchymal stem cells.

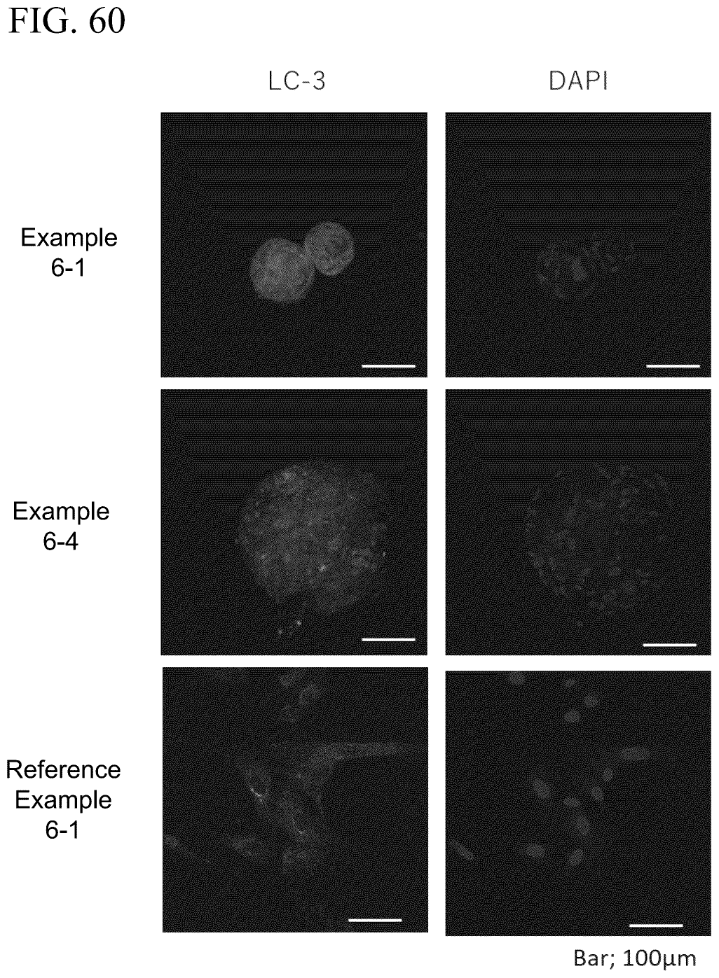

FIG. 60 shows micrographs showing aspects of the expression of an autophagy-related factor LC-3 in the mesenchymal stem cells (spheroids) within the hydrogel fibers in Examples 6-1 and 6-4. FIG. 60 shows the results of analyzing the LC-3 expression in the spheroids within the hydrogel fibers on Day 14 since the hydrogel fiber preparation or in the cells subjected to the 2-dimensional culture.

The images on the left side in FIG. 60 are obtained by performing immunofluorescence cell staining using a primary antibody against LC-3 (anti-LC3 monoclonal antibody, manufactured by MBL International Corporation, No. M152-3) and a fluorescently labeled secondary antibody (Fluoro-conjugated Goat anti-Rabbit-IgG antibody, manufactured by Merck KGaA, AP187F) and performing observation with a confocal laser scanning microscope. Green fluorescence is detected, and the part in which the green fluorescence is detected indicates the presence of the autophagy-related factor LC-3. The images on the right side in FIG. 60 are the results of DAPI staining, and the fluorescent part indicates the site of the nucleus (DNA) of the viable cell.

In both Examples 6-1 and 6-4, LC-3 is expressed mainly in viable cells in the vicinity of the surfaces of the spheroids. The LC-3 expression on the surfaces of the spheroids in Examples 6-1 and 6-4 is stronger than the LC-3 expression in Reference Example 6-1.

This indicates that the viable cells are localized on the surface of the spheroid within the hydrogel structure, and autophagy is induced in these viable cells. It is considered that such promotion of autophagy enables the long-term survival of the mesenchymal stem cells.

[Hydrogel Fiber Characteristic Analysis (11)]

### Example 7-1

A hydrogel structure according to Example 7-1 has a shape different from the fiber shape. In Example 7-1, the hydrogel fiber described in Example 6-1 was prepared first. On Day 5 since the preparation of the hydrogel fiber according to Example 6-1, a hydrogel fiber 10 was wound around a glass tube 30, and a second hydrogel 22 was formed so as to cover the entire wound hydrogel fiber 20 (refer to FIGS. 61 and 62). Here, the second hydrogel was an alginate gel. In this manner, the hydrogel structure according to Example 7-1 was produced.

The mesenchymal stem cells encapsulated in the hydrogel structure according to Example 7-1 were cultured in a state of being wound around the glass tube 30. FIG. 62 is an enlarged view of a micrograph acquired on Day 9 since the start of the culture.

### Example 7-2

A hydrogel structure according to Example 7-2 was produced in the same manner as in Example 7-1, except that shaping was performed using the hydrogel fiber described in Example 6-2. Thus, the hydrogel structure according to Example 7-2 was produced in the same manner as in Example 7-1, except for the number of cells encapsulated in the original hydrogel fiber (initial cell density).

### Example 7-3

A hydrogel structure according to Example 7-3 was produced in the same manner as in Example 7-1, except that shaping was performed using the hydrogel fiber described in Example 6-3. Thus, the hydrogel structure according to Example 7-3 was produced in the same manner as in Example 7-1, except for the number of cells encapsulated in the original hydrogel fiber (initial cell density).

TABLE 4

| | | Tissue of origin | Cell density | Base | | Shape of |
|---|---|---|---|---|---|---|
| | Cells | of cells | (cells/mL) | Material | Hydrogel | structure |
| Example 6-1 | MSC | Umbilical cord | 1 × 10⁸ | Medium | Calcium alginate | string shape |
| Example 6-2 | MSC | Umbilical cord | 5 × 10⁷ | Medium | Calcium alginate | string shape |
| Example 6-3 | MSC | Umbilical cord | 1 × 10⁷ | Medium | Calcium alginate | string shape |
| Example 7-1 | MSC | Umbilical cord | 1 × 10⁸ | Medium | Calcium alginate | coil shape |
| Example 7-2 | MSC | Umbilical cord | 5 × 10⁷ | Medium | Calcium alginate | coil shape |
| Example 7-3 | MSC | Umbilical cord | 1 × 10⁷ | Medium | Calcium alginate | coil shape |

Next, the cases of immersing and culturing the mesenchymal stem cells encapsulated in the hydrogel fibers in Examples 6-1 to 6-3 and 7-1 to 7-3 in the medium together with the fibers were compared with the case of performing 2-dimensional culture of the mesenchymal stem cells without encapsulating the mesenchymal stem cells in the hydrogel fibers (Reference Example 6-1). Specifically, the amounts of various humoral factors secreted into the medium, various expression factors related to mRNA, and the like were measured.

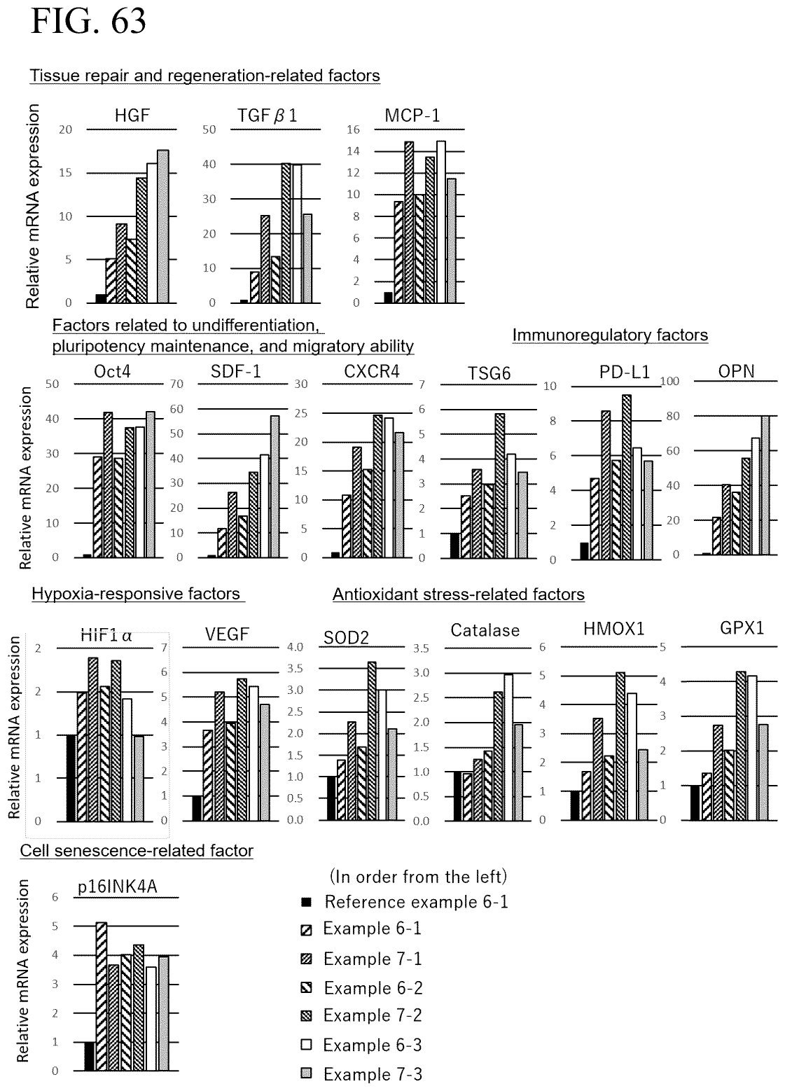

FIG. 63 shows graphs of the results of measuring various expression factors related to the mRNA of the mesenchymal stem cells constituting the hydrogel structures in Examples 6-1 to 6-3 and 7-1 to 7-3. The vertical axes in FIG. 63 represent the ratios obtained when the value for the mesenchymal stem cells subjected to the 2-dimensional culture (Reference Example 6-1) was normalized as “1”. FIG. 63 shows the measurement results obtained when 16 days have passed since the start of the culture.

FIG. 63 shows tissue repair and regeneration-related factors (HGF, TGFβ, and MCP-1), undifferentiation/pluripotency maintenance/cellular motility-related factors (Oct-4, SDF-1, and CXCR4), immunoregulatory factors (TSG6, PD-L1, and OPN), hypoxia-responsive factors (HIF1α and VEGF), antioxidant stress-related factors (SOD2, Catalase, HMOX1, and GPX1), and a cell senescence-related factor and cancer-related gene (p16INK4A).

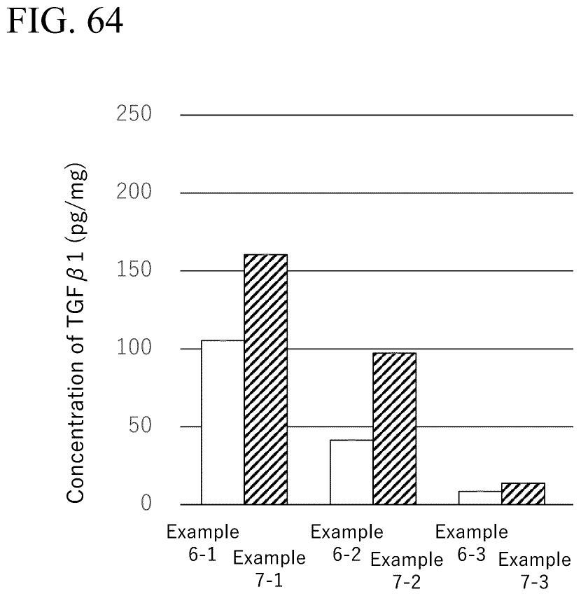

FIG. 64 shows a graph of the results of measuring a humoral factor (TGF-β1) secreted from the mesenchymal stem cells constituting the hydrogel structures in Examples 6-1 to 6-3 and 7-1 to 7-3. The vertical axis in FIG. 64 represents the concentration of TGF-β1 in the total proteins in the medium. When the day on which the hydrogel structure was prepared was set to Day 0, TGF-β1 was measured on Day 7.

It was found from FIG. 64 that the amounts of TGFβ1 secreted in the hydrogel structures which were shaped into a coil shape (Examples 7-1 to 7-3) tended to be greater than the amounts of TGFβ1 secreted in the fibrous hydrogel structures (Example 6-1 to 6-3), regardless of the initial cell density.

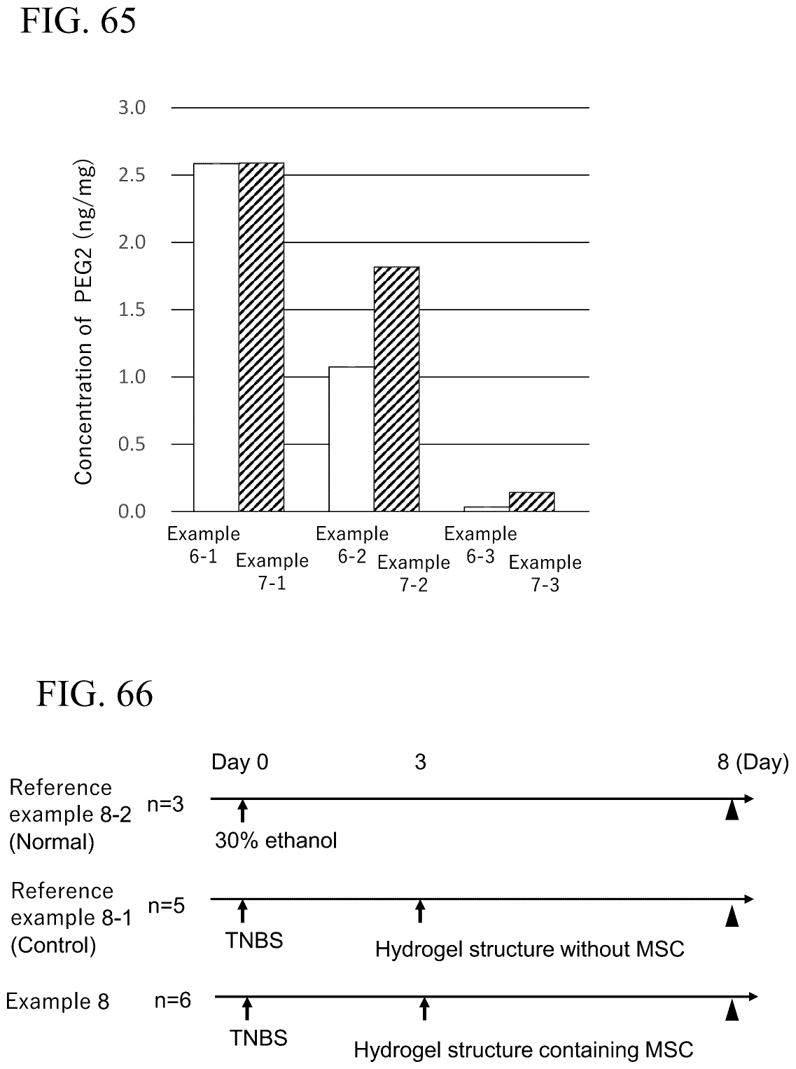

FIG. 65 shows a graph of the results of measuring a humoral factor (prostaglandin E2; PGE2) secreted from the mesenchymal stem cells constituting the hydrogel structures in Examples 6-1 to 6-3 and 7-1 to 7-3.

The vertical axis in FIG. 65 represents the concentration of PGE2 in the total proteins in the medium. When the day on which the hydrogel structure was prepared was set to Day 0, PGE2 was measured on Day 7.

As can be seen in Examples 6-1 and 7-1, the amounts of PGE2 secreted were equal in the hydrogel structures in which the initial cell densities were high, regardless of the shape of the hydrogel structure. In addition, it was found that, if the initial cell density was medium or lower, the amounts of PGE2 secreted in the hydrogel structures that were shaped into a coil shape (Examples 7-2 and 7-3) tended to be greater than the amounts of PGE2 secreted in the fibrous hydrogel structures (Examples 6-2 and 6-3).

[Application to TNBS Enteritis Model Rats]

### Example 8

A hydrogel structure according to Example 8 and a production method therefor will be described. The hydrogel structure according to Example 8 was produced in the same manner as in Example 7-1, except that shaping was performed using the hydrogel fiber described in Example 6-4. Thus, the hydrogel structure according to Example 8 was produced in the same manner as in Example 7-1, except that the core solution used as the base material during the hydrogel fiber production contained atelocollagen. Therefore, the hydrogel structure according to Example 8 has the same coil shape as that in Example 7-1.

As Reference Example 8-1, a hydrogel structure not encapsulating the mesenchymal stem cells was prepared. The hydrogel structure according to Reference Example 8-1 was produced in the same manner as in Example 8, except that the mesenchymal stem cells were not introduced into the hydrogel fiber. Therefore, the hydrogel structure according to Reference Example 8-1 has the same shape as that in Example 8.

Next, an experiment in which the hydrogel structure according to Example 8 was applied to the treatment of TNBS enteritis model rats and results thereof will be described. FIG. 66 is a diagram for describing schedules for the treatment by the hydrogel structure in Example 8 using the TNBS enteritis model rats.

First, a TNBS enteritis model was prepared by performing transanal enema administration of TNBS to SD rats (male, 14 weeks old). First, TNBS enteritis model rats were prepared by performing transanal enema administration of an ethanol solution in which 2,4,6-trinitrobenzenesulfonic acid (TNBS) was dissolved. Transanal enema administration of the hydrogel structure according to Example 8 or Reference Example 8-1 was performed on 3 days after the induction of enteritis (administration of TNBS). Since the induction of enteritis (administration of TNBS), body weight changes and disease activities (DAIs) of the model rats were continuously observed.

In Reference Example 8-2, SD rats (male, 14 weeks old) were prepared by subjecting the rats to transanal enema administration of 30% ethanol on the same day as the TNBS administration, and body weight changes and disease activities (DAIs) of the model rats were continuously observed (normal group).

The value of “n” in FIG. 66 indicates the number of model rat samples used in each Example and Reference Example.

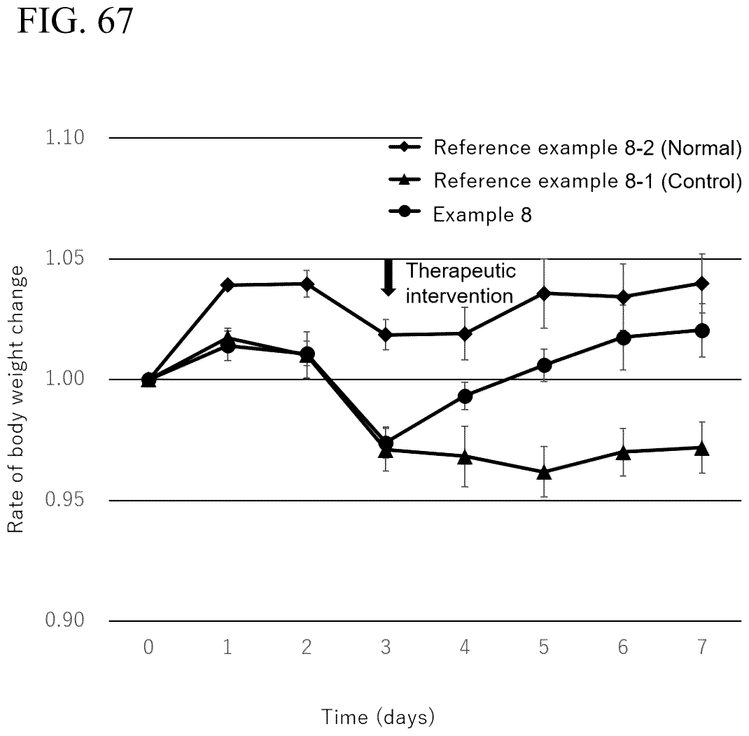

FIG. 67 shows a graph of body weight changes in the model rats that have received the various treatments. The vertical axis in FIG. 67 represents numerical values obtained by correcting the body weight of each individual during the observation period by the body weight on Day 0, thus setting the body weight of the model rat on Day 0 to “1”, whereby normalization was performed so that the Rate of body weight change was the same in each Example and Reference Example.

Since exacerbation of enteritis is accompanied by symptoms of diarrhea and hematochezia in the model rats, the body weights of the model rats decrease. Therefore, the body weights of the model rats in Example 8 and Reference Example 8-1 become lower than the body weights of the model rats in the normal group in Reference Example 8-2 with the passage of days.

After the administration of the hydrogel structure, the rate of body weight change of the model rats in Example 8 was maintained at higher values than the rate of body weight change of the model rats in Reference Example 8-1. In other words, it is considered that the symptoms of enteritis were alleviated in the model rats into which the hydrogel structure encapsulating the mesenchymal stem cells was transplanted.

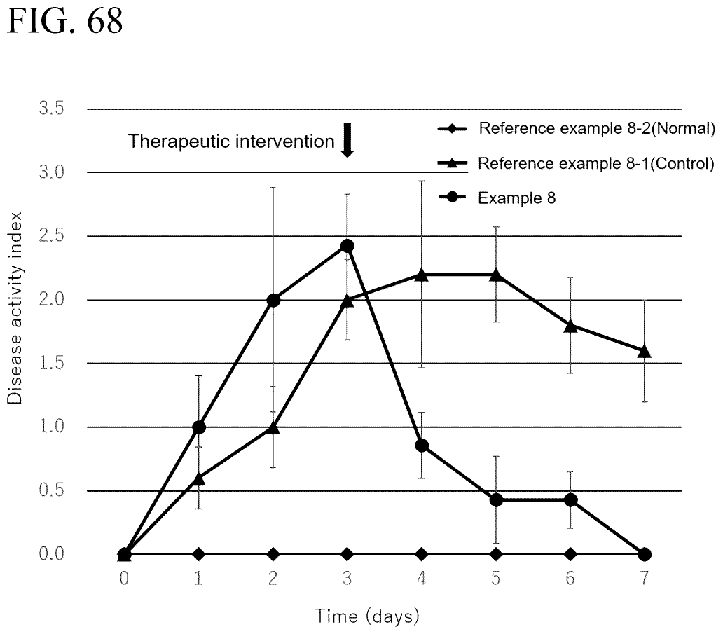

FIG. 68 shows a graph of disease activity indices (DAIs) for the TNBS enteritis model rats that have received the various treatments. DAI is an index of enteritis activity obtained by scoring the rate of body weight loss, diarrhea, and the state of hematochezia in the model rats. The method for evaluating the DAI is as described above.

With the exacerbation of enteritis, the DAI for the model rats in Reference Example 8-1 becomes higher than the DAI for the model rats in Reference Example 8-2, which is the normal group. Note that the DAIs of the model rats in Reference Example 8-2, which is the normal group, were almost “0”.

It is found that, in the model in Example 8, the administration of the hydrogel structure reduces the DAI at an early stage. It is thus found that the administration of the hydrogel structure encapsulating the mesenchymal stem cells acts effectively on the TNBS enteritis model rats.

Next, the model rats were dissected 8 days after the administration of TNBS or ethanol. In this manner, the gross appearance in the peritoneal cavity associated with intestinal inflammation, the major axis of the intestine, the intestinal weight, and the percentage of the area of the grossly observed lesion were evaluated.

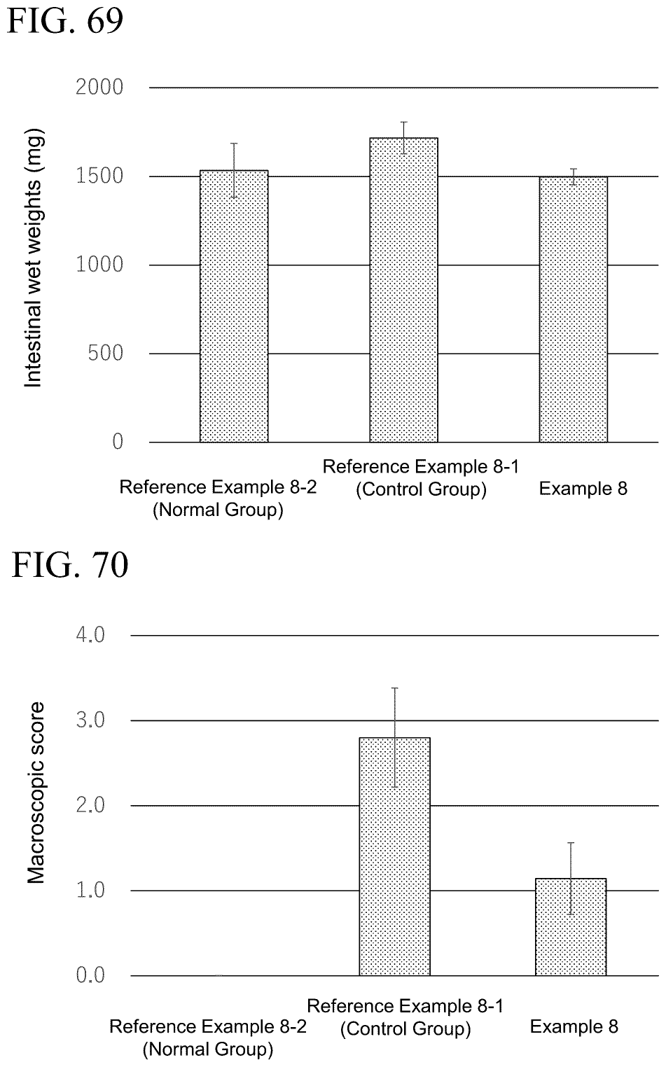

FIG. 69 shows a graph of intestinal wet weights in the model rats that have been dissected 8 days after the administration of TNBS or ethanol.

When Reference Example 8-2 (normal group) was compared with Reference Example 8-1 (control group), the intestinal wet weight of the model rats in Reference Example 8-1 was greater than the intestinal wet weight of the model rats in Reference Example 8-2. This is considered to be due to the influence of the intestinal inflammation.

The intestinal wet weight of the model rats in Example 8 was smaller than the intestinal wet weight of the model rats in Reference Example 8-1. This is considered to be due to the suppression of wall thickening which accompanies the intestinal inflammation.

FIG. 70 shows a graph of gross appearance scores of external appearances of intestines in peritoneal cavities of the TNBS enteritis model rats that have received the various treatments. For the purpose of evaluating the degree of the influence of inflammation on the serosa side of the intestinal wall that accompanies TNBS enteritis, the gross appearance in the peritoneal cavity (so-called the observation of the external appearance of the intestine) was scored. The scores were evaluated from macro images acquired when the model rats were dissected.

The evaluation method is as follows. First, the degrees of “vascularity images”, “wall thickening”, and “adhesion of surrounding tissue” in the intestinal wall were each evaluated as follows in 4 stages of 0,1,2, and 3 (refer to Martin Arranz et al. Stem Cell Research & Therapy (2018) 9:95).
1) Vascularity
Normal: 0 points
Mild vascular pattern distortion: 1 point
Severe vascular pattern distortion: 2 points
Complete lack of vascular pattern: 3 points
2) Wall thickening
Normal: 0 points
Mild: 1 point
Severe: 2 points
Very severe: 3 points
3) Adhesion of surrounding tissue
None: 0 points
Mild adhesion: 1 point
Moderate adhesion: 2 points
Severe adhesion: 3 points

The total of the above points was defined as the gross appearance score in the peritoneal cavity. Here, the lower the numerical value of the gross appearance score, the closer the rat is to the normal condition.

From FIG. 70, it is found that the gross appearance score in Reference Example 8-1 (control group) is higher than the gross appearance score in Reference Example 8-2 (normal group). In addition, the gross appearance score in Example 8 is lower than the gross appearance score in Reference Example 8-1 (control group). It is thus found that the symptoms in the model rats in Example 8 have been improved more than those in Reference Example 8-1.

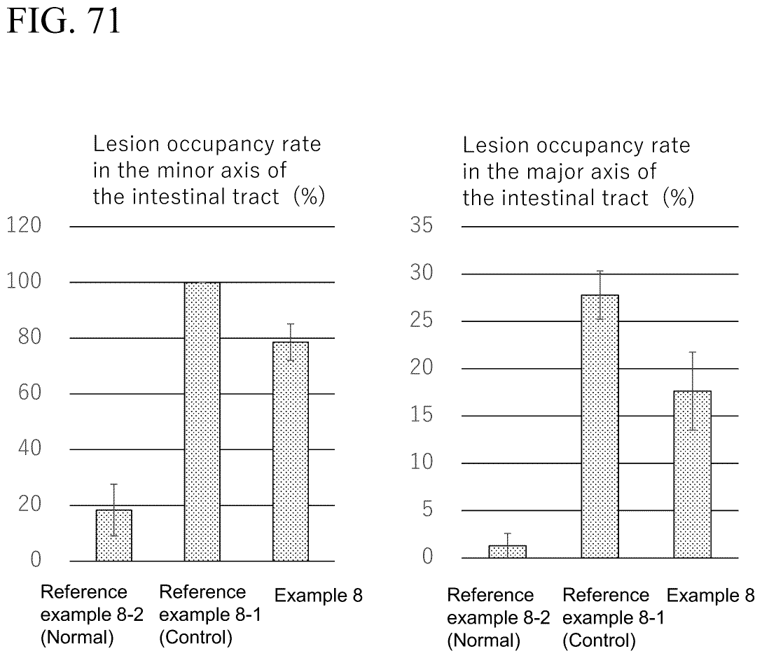

FIG. 71 shows graphs of gross lesion occupancy evaluation in mucosal surfaces (internal appearances) of the intestines of the TNBS enteritis model rats that have received the various treatments. For the purpose of evaluating the occupancy rate of a lesion in the mucosal surface that accompanies TNBS enteritis, the occupancy of the lesion was measured (so-called the observation of the internal appearance of the intestine).

The occupancy of the lesion was evaluated by macro images of the intestines that were resected during the dissection of the model rats and opened by incision in the longitudinal direction (direction along the intestine). The occupancy of the lesion in the minor axis direction was defined by a value (%) obtained by multiplying, by 100, the value obtained by dividing the length of the largest lesion site in the minor axis direction of the intestine by the length of the intestine in the minor axis direction. The occupancy of the lesion in the major axis direction was defined by a value (%) obtained by multiplying, by 100, the value obtained by dividing the length of the lesion site in the total length of the proximal colon excluding the length from the anus to the cecum by the total length of the proximal colon excluding the length from the anus to the cecum.

From FIG. 71, it is found that the occupancy of the lesion in Reference Example 8-1 (control group) was higher than the occupancy of the lesion in Reference Example 8-2 (normal group) in both the minor axis direction and the major axis direction. In addition, the occupancy of the lesion in Example 8 was lower than the occupancy of the lesion in Reference Example 8-1 (control group). It is thus found that the symptoms in the model rats in Example 8 have been improved more than those in Reference Example 8-1.

It can be understood that at least the following inventions are specified in the present specification based on the above-described embodiments and/or examples and the following additional descriptions.

### APPENDIX 1

A hydrogel structure including a hydrogel with fiber shape, the hydrogel encapsulating mesenchymal stem cells.

### APPENDIX 2

The hydrogel structure according to Appendix 1, wherein the hydrogel structure includes: the hydrogel; and a base material and the mesenchymal stem cells that are provided inside the hydrogel.

### APPENDIX 3

A hydrogel structure including: a base material containing mesenchymal stem cells; and a hydrogel encapsulating the base material.

### APPENDIX 4

The hydrogel structure according to Appendix 2 or 3, wherein the base material contains collagen, laminin, fibronectin or a liquid medium, or a combination thereof.

### APPENDIX 5

The hydrogel structure according to any one of Appendices 1 to 4, wherein the mesenchymal stem cells are umbilical cord-derived, placenta-derived, bone marrow-derived, amnion-derived, dental pulp-derived or adipose-derived mesenchymal stem cells.

### APPENDIX 6

The hydrogel structure according to any one of Appendices 1 to 5, wherein the hydrogel contains calcium alginate or barium alginate.

### APPENDIX 7

The hydrogel structure according to any one of Appendices 1 to 6, wherein the mesenchymal stem cells form a spheroid and maintains a differentiation potential.

### APPENDIX 8

A hydrogel structure comprising:
A form shaped by the hydrogel structure according to any one of Appendices 1 to 7; and
a second hydrogel covering the form.

### APPENDIX 9

The hydrogel structure according to Appendix 8, wherein the form contains the fibrous hydrogel that is regularly shaped.

### APPENDIX 10

The hydrogel structure according to Appendix 8 or 9, wherein the form contains the fibrous hydrogel that is formed into a spiral shape, a grid shape, a lattice shape and/or a mesh shape.

### APPENDIX 11

The hydrogel structure according to any one of Appendices 1 to 10, wherein the hydrogel structure is for regulation of gene expression of a factor that is expressed by the mesenchymal stem cells.

### APPENDIX 12

The hydrogel structure according to any one of Appendices 1 to 11, wherein the hydrogel structure is for transplantation.

### APPENDIX 13

The hydrogel structure according to any one of Appendices 1 to 12, wherein the hydrogel structure is for at least one of suppression of fibrogenesis, suppression of inflammatory cell infiltration, tissue repair and regeneration, and suppression of an inflammatory cytokine.

### APPENDIX 14

The hydrogel structure according to any one of Appendices 1 to 13, wherein the hydrogel structure is for treating enteritis or preventing enteritis.

### APPENDIX 15

A culture supernatant that is obtained from a culture medium in which the mesenchymal stem cells are cultured in a state of being encapsulated in the hydrogel structure according to any one of Appendices 1 to 14.

### APPENDIX 16

An enhancing agent comprising the hydrogel structure according to any one of Appendices 1 to 14, wherein the enhancing agent is for expression of a hypoxia-responsive factor by the mesenchymal stem cells contained in the hydrogel structure.

### APPENDIX 17

An enhancing agent comprising the hydrogel structure according to any one of Appendices 1 to 14, wherein the enhancing agent is for expression of an antioxidant stress-related factor by the mesenchymal stem cells contained in the hydrogel structure.

### APPENDIX 18

A macrophage activity inhibitor comprising the hydrogel structure according to any one of Appendices 1 to 14 or the culture supernatant according to Appendix 15.

### APPENDIX 19

An agent for treating enteritis or for preventing enteritis, the agent comprising a supernatant of a culture medium in which the mesenchymal stem cells are cultured in a state of being encapsulated in the hydrogel structure according to any one of Appendices 1 to 14.

### APPENDIX 20

An application method comprising:
applying the hydrogel structure according to any one of Appendices 1 to 14 inside a biological body or onto a surface of the biological body.

### APPENDIX 21

A topical agent comprising the hydrogel structure according to any one of Appendices 1 to 14.

### APPENDIX 22

A method for producing a hydrogel structure, the method including: mixing mesenchymal stem cells and a base material; and embedding the mixture in a hydrogel.

Although the content of the present invention has been disclosed through embodiments and examples as described above, the statements and drawings forming part of this disclosure should not be construed as limiting the present invention. Various alternative embodiments, examples, and operational techniques will become apparent to those skilled in the art from this disclosure. Therefore, the technical scope of the present invention is defined only by the matters specifying the invention according to the valid claims based on the above descriptions.

The present application claims priority based on Japanese Patent Application No. 2020-183302 filed on Oct. 30, 2020, and the entire contents of the patent application are incorporated herein by reference.

1. A hydrogel structure comprising:
a base material containing mesenchymal stem cells; and
a tubular hydrogel encapsulating the base material,
wherein the mesenchymal stem cells form a spheroid and maintain a differentiation potential.

2. The hydrogel structure according to claim 1, wherein the spheroid is formed by the mesenchymal stem cells that are aggregated within the hydrogel.

3. The hydrogel structure according to claim 1, wherein
viable mesenchymal stem cells are located on a surface of the spheroid.

4. The hydrogel structure according to claim 1, comprising extracellular matrix and/or collagen into the spheroid.

5. The hydrogel structure according to claim 1, wherein a storage modulus (G′) of the hydrogel structure is 100 Pa or more at a frequency of 1 Hz.

6. The hydrogel structure according to claim 1, wherein the base material contains collagen, laminin, fibronectin or a liquid medium, or a combination thereof.

7. The hydrogel structure according to claim 1, wherein the hydrogel contains calcium alginate or barium alginate.

8. A hydrogel structure according to claim 1, comprising:
a form shaped by the tubular hydrogel; and
a second hydrogel covering the form.

9. A Graft comprising the hydrogel structure according to claim 1.

10. A method, comprising
regulating gene expression of factors expressed by mesenchymal stem cells by using the hydrogel structure according to claim 1.

11. A method for suppressing fibrosis, for suppressing inflammatory cell infiltration, for tissue repair and regeneration, for suppressing inflammatory cytokines, for treating enteritis, or for preventing enteritis, comprising
applying the hydrogel structure according to claim 1 to a cell, a tissue or a biological body.

12-14. (canceled)

15. A culture supernatant obtained from a culture medium in which the mesenchymal stem cells encapsulated in the hydrogel structure according claim 1 are cultured.

16-17. (canceled)

18. A method for suppressing macrophage activity, comprising:
applying the hydrogel structure according to claim 1 or the culture supernatant obtained from a culture medium in which the mesenchymal stem cells encapsulated in the hydrogel structure according to claim 1 are cultured to macrophage or inflammatory cell.

19. A method for treating enteritis or for preventing enteritis, comprising
applying the culture supernatant according to claim 15 to a biological body.

20. (canceled)

21. A topical agent comprising the hydrogel structure according to claim 1.

22. A method for producing a hydrogel structure, the method comprising:
foaming a spheroid of the mesenchymal stem cells by culturing mesenchymal stem cells within a tubular hydrogel that encapsulates a base material containing the mesenchymal stem cells.

23. The method for producing a hydrogel structure according to claim 22, comprising:
forming a first laminar flow of a cell suspension containing the mesenchymal stem cells and the base material;
forming a second laminar flow of a hydrogel preparation solution that covers an outer perimeter of the first laminar flow; and
forming the tubular hydrogel by turning the hydrogel preparation solution into gel.

24. A method for producing a spheroid, the method comprising:
forming a spheroid of the mesenchymal stem cells by culturing mesenchymal stem cells within a tubular hydrogel that encapsulates a base material containing the mesenchymal stem cells.

25. The method for producing a spheroid according to claim 24, comprising
enhancing an expression of a hypoxia-responsive factor or an antioxidant stress-related factor by mesenchymal stem cells during forming the spheroids.

26. A spheroid formed by culturing mesenchymal stem cells within a tubular hydrogel that encapsulates a base material containing the mesenchymal stem cells.
